SEQUENCE LISTING (243 sequences)

SEQ ID NO: 1 - DNA, 354 bp, Homo sapiens
  source 1..354: mol_type = other DNA
gaagtgcagctggtggagtctgggggaggcttggtgcagcctggcaggtccctgagactctcctgtgcggcctctggattcacctttgatgattatgccatacactgggtccggcaagctccagggaagggcctggagtgggtctcagttatcagttggaatagtgatatcataggctatgcggactctgtgaagggccgattcaccgtctccagagacaacgccaagaactccctgtatctgcaaatgaatagtctgagaactgaggacacggccttgtattactgtgcaaaaggatataactggaacttctttgactattggggccagggaaccctggtcaccgtctcctca

SEQ ID NO: 2 - protein, 118 aa, Homo sapiens
EVQLVESGGGLVQPGRSLRLSCAASGFTFDDYAIHWVRQAPGKGLEWVSVISWNSDIIGYADSVKGRFTVSRDNAKNSLYLQMNSLRTEDTALYYCAKGYNWNFFDYWGQGTLVTVSS

SEQ ID NO: 3 - DNA, 24 bp, Homo sapiens
  source 1..24: mol_type = other DNA
ggattcacctttgatgattatgcc

SEQ ID NO: 4 - protein, 8 aa, Homo sapiens
GFTFDDYA

SEQ ID NO: 5 - DNA, 24 bp, Homo sapiens
  source 1..24: mol_type = other DNA
atcagttggaatagtgatatcata

SEQ ID NO: 6 - protein, 8 aa, Homo sapiens
ISWNSDII

SEQ ID NO: 7 - DNA, 33 bp, Homo sapiens
  source 1..33: mol_type = other DNA
gcaaaaggatataactggaacttctttgactat

SEQ ID NO: 8 - protein, 11 aa, Homo sapiens
AKGYNWNFFDY

SEQ ID NO: 9 - DNA, 321 bp, Homo sapiens
  source 1..321: mol_type = other DNA
gaaattgtgttgacgcagtctccagccaccctgtctttatctccaggggaaagagccaccctctcctgcagggccagtcagagtgttagcagctacttagcctggtaccaacagaaacctggccaggctcccaggctcctcatctataatgcagcaaacagggccactgacatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 10 - protein, 107 aa, Homo sapiens
EIVLTQSPATLSLSPGERATLSCRASQSVSSYLAWYQQKPGQAPRLLIYNAANRATDIPARFSGSGSGTDFTLTISSLEPEDFAVYYCQQRSNWPLTFGGGTKVEIK

SEQ ID NO: 11 - DNA, 18 bp, Homo sapiens
  source 1..18: mol_type = other DNA
cagagtgttagcagctac

SEQ ID NO: 12 - protein, 6 aa, Homo sapiens
QSVSSY

SEQ ID NO: 13-14 - skipped (2 entries)

SEQ ID NO: 15 - DNA, 27 bp, Homo sapiens
  source 1..27: mol_type = other DNA
cagcagcgtagcaactggcctctcact

SEQ ID NO: 16 - protein, 9 aa, Homo sapiens
QQRSNWPLT

SEQ ID NO: 17 - DNA, 354 bp, Homo sapiens
  source 1..354: mol_type = other DNA
gaagtgcagctggtggagtctgggggaggcttggtacagcctggcaggtccctgagactctcctgtgcagcctctggattcacctttgatgattatgccatgcactgggtccggcaaactccagggaagggcctggagtgggtctcagttattagttggaatagtgatgtcatagcctattcggactctgtgaagggccgcttcaccatttccagagacaacgccaagaactccctgtatctgcaaatgaacagtctgggaactgaggacacggccttatattactgtgcaaaaggccataactggaacttctttgactattggggccagggaaccctggtcaccgtctcctca

SEQ ID NO: 18 - protein, 118 aa, Homo sapiens
EVQLVESGGGLVQPGRSLRLSCAASGFTFDDYAMHWVRQTPGKGLEWVSVISWNSDVIAYSDSVKGRFTISRDNAKNSLYLQMNSLGTEDTALYYCAKGHNWNFFDYWGQGTLVTVSS

SEQ ID NO: 19 - DNA, 24 bp, Homo sapiens
  source 1..24: mol_type = other DNA
ggattcacctttgatgattatgcc

SEQ ID NO: 20 - protein, 8 aa, Homo sapiens
GFTFDDYA

SEQ ID NO: 21 - DNA, 24 bp, Homo sapiens
  source 1..24: mol_type = other DNA
attagttggaatagtgatgtcata

SEQ ID NO: 22 - protein, 8 aa, Homo sapiens
ISWNSDVI

SEQ ID NO: 23 - DNA, 33 bp, Homo sapiens
  source 1..33: mol_type = other DNA
gcaaaaggccataactggaacttctttgactat

SEQ ID NO: 24 - protein, 11 aa, Homo sapiens
AKGHNWNFFDY

SEQ ID NO: 25 - DNA, 321 bp, Homo sapiens
  source 1..321: mol_type = other DNA
gaaattgtgttgacacagtctccagccaccctgtctttgtctccaggagaaagagccaccctctcctgcagggccagtcagagtgttagcagctacttagcctggtaccaacagaaacctggccaggctcccaggctcctcatctataatgtagccaacagggccacagacatcccagccaggttcagtggcagtgggtctgggacagacttcactctcaccatcagcggcctagagcctgaagattttgcagtttatttctgtcagcagcgtagcaactggcctctcactttcggcggagggaccaaggtggagatcaaa

SEQ ID NO: 26 - protein, 107 aa, Homo sapiens
EIVLTQSPATLSLSPGERATLSCRASQSVSSYLAWYQQKPGQAPRLLIYNVANRATDIPARFSGSGSGTDFTLTISGLEPEDFAVYFCQQRSNWPLTFGGGTKVEIK

SEQ ID NO: 27 - DNA, 18 bp, Homo sapiens
  source 1..18: mol_type = other DNA
cagagtgttagcagctac

SEQ ID NO: 28 - protein, 6 aa, Homo sapiens
QSVSSY

SEQ ID NO: 29-30 - skipped (2 entries)

SEQ ID NO: 31 - DNA, 27 bp, Homo sapiens
  source 1..27: mol_type = other DNA
cagcagcgtagcaactggcctctcact

SEQ ID NO: 32 - protein, 9 aa, Homo sapiens
QQRSNWPLT

SEQ ID NO: 33 - DNA, 354 bp, Homo sapiens
  source 1..354: mol_type = other DNA
gaagtgcagctggtggagtctgggggaggcttggtacagcctggcaggtccctgagactctcctgtacagcctctggattcacctttgatgattatgccatacactgggtccggcaatctccagggaagggcctggagtgggtctcagttatcagttggaatagtgatgtcataggctatgcggactctgtgaagggccgattcaccatctccagagacaacgccaagaactccctgtatctgcagatgaatagtctgagagctgaggacacggccttgtattactgtgcaaaaggatataactggaacttctttgactattggggccagggaaccctggtcaccgtctcctca

SEQ ID NO: 34 - protein, 118 aa, Homo sapiens
EVQLVESGGGLVQPGRSLRLSCTASGFTFDDYAIHWVRQSPGKGLEWVSVISWNSDVIGYADSVKGRFTISRDNAKNSLYLQMNSLRAEDTALYYCAKGYNWNFFDYWGQGTLVTVSS

SEQ ID NO: 35 - DNA, 24 bp, Homo sapiens
  source 1..24: mol_type = other DNA
ggattcacctttgatgattatgcc

SEQ ID NO: 36 - protein, 8 aa, Homo sapiens
GFTFDDYA

SEQ ID NO: 37 - DNA, 24 bp, Homo sapiens
  source 1..24: mol_type = other DNA
atcagttggaatagtgatgtcata

SEQ ID NO: 38 - protein, 8 aa, Homo sapiens
ISWNSDVI

SEQ ID NO: 39 - DNA, 33 bp, Homo sapiens; identical to SEQ ID NO: 7
  source 1..33: mol_type = other DNA

SEQ ID NO: 40 - protein, 11 aa, Homo sapiens
AKGYNWNFFDY

SEQ ID NO: 41 - DNA, 321 bp, Homo sapiens; identical to SEQ ID NO: 9
  source 1..321: mol_type = other DNA

SEQ ID NO: 42 - protein, 107 aa, Homo sapiens; identical to SEQ ID NO: 10

SEQ ID NO: 43 - DNA, 18 bp, Homo sapiens
  source 1..18: mol_type = other DNA
cagagtgttagcagctac

SEQ ID NO: 44 - protein, 6 aa, Homo sapiens
QSVSSY

SEQ ID NO: 45-46 - skipped (2 entries)

SEQ ID NO: 47 - DNA, 27 bp, Homo sapiens
  source 1..27: mol_type = other DNA
cagcagcgtagcaactggcctctcact

SEQ ID NO: 48 - protein, 9 aa, Homo sapiens
QQRSNWPLT

SEQ ID NO: 49 - DNA, 354 bp, Homo sapiens
  source 1..354: mol_type = other DNA
gaagtgcagctggtggagtctgggggagacttggtacagcctggcaggtccctgagactctcctgtgcagcctctggattcacctttgatgattatgccatgcactgggtccggcaagctccagggaagggcctggaatgggtctcagttattagttggaatagtgatgtcatagcctattcggactctgtgaagggccgattcaccatctccagagacaacgccaagaactccctgtatctgcaaatgaacagtctgagaactgaggacacggccttatattactgtacaaaaggccataagtggagcttctttgactattggggccagggaaccctggtcaccgtctcctca

SEQ ID NO: 50 - protein, 118 aa, Homo sapiens
EVQLVESGGDLVQPGRSLRLSCAASGFTFDDYAMHWVRQAPGKGLEWVSVISWNSDVIAYSDSVKGRFTISRDNAKNSLYLQMNSLRTEDTALYYCTKGHKWSFFDYWGQGTLVTVSS

SEQ ID NO: 51 - DNA, 24 bp, Homo sapiens
  source 1..24: mol_type = other DNA
ggattcacctttgatgattatgcc

SEQ ID NO: 52 - protein, 8 aa, Homo sapiens
GFTFDDYA

SEQ ID NO: 53 - DNA, 24 bp, Homo sapiens
  source 1..24: mol_type = other DNA
attagttggaatagtgatgtcata

SEQ ID NO: 54 - protein, 8 aa, Homo sapiens
ISWNSDVI

SEQ ID NO: 55 - DNA, 33 bp, Homo sapiens
  source 1..33: mol_type = other DNA
acaaaaggccataagtggagcttctttgactat

SEQ ID NO: 56 - protein, 11 aa, Homo sapiens
TKGHKWSFFDY

SEQ ID NO: 57 - DNA, 321 bp, Homo sapiens
  source 1..321: mol_type = other DNA
gaaattgtgttgacacagtctccagccaccctgtctttgtctccaggggaaagagccaccctctcctgcagggccagtcagagtattagcagctacttagcctggtaccaacagaaacctggccaggctcccagactcctcatctttaatgtagccaacagggccactgacatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 58 - protein, 107 aa, Homo sapiens
EIVLTQSPATLSLSPGERATLSCRASQSISSYLAWYQQKPGQAPRLLIFNVANRATDIPARFSGSGSGTDFTLTISSLEPEDFAVYYCQQRSNWPLTFGGGTKVEIK

SEQ ID NO: 59 - DNA, 18 bp, Homo sapiens
  source 1..18: mol_type = other DNA
cagagtattagcagctac

SEQ ID NO: 60 - protein, 6 aa, Homo sapiens
QSISSY

SEQ ID NO: 61-62 - skipped (2 entries)

SEQ ID NO: 63 - DNA, 27 bp, Homo sapiens
  source 1..27: mol_type = other DNA
cagcagcgtagcaactggcctctcact

SEQ ID NO: 64 - protein, 9 aa, Homo sapiens
QQRSNWPLT

SEQ ID NO: 65 - DNA, 339 bp, Homo sapiens
  source 1..339: mol_type = other DNA
gaggtgcagctggtggagtctgggggaggcttggttcagcctggggggtccctgagactctcctgcgcagcctctggattcacctttagcgactatgccatgagctgggtccgccaggctccggggaaggggctggagtgggtctcaggtattagtggaaatggtggtgacacatactacggagacttcgtgaagggccggttcaccatctccagagacaattccaagaacacgctgtatctgcaaatgaacagcctgagaggcgaggacacggccgcatatttctgtgtgatagatcttgactattggggtcagggaaccctggtcaccgtctcctca

SEQ ID NO: 66 - protein, 113 aa, Homo sapiens
EVQLVESGGGLVQPGGSLRLSCAASGFTFSDYAMSWVRQAPGKGLEWVSGISGNGGDTYYGDFVKGRFTISRDNSKNTLYLQMNSLRGEDTAAYFCVIDLDYWGQGTLVTVSS

SEQ ID NO: 67 - DNA, 24 bp, Homo sapiens
  source 1..24: mol_type = other DNA
ggattcacctttagcgactatgcc

SEQ ID NO: 68 - protein, 8 aa, Homo sapiens
GFTFSDYA

SEQ ID NO: 69 - DNA, 24 bp, Homo sapiens
  source 1..24: mol_type = other DNA
attagtggaaatggtggtgacaca

SEQ ID NO: 70 - protein, 8 aa, Homo sapiens
ISGNGGDT

SEQ ID NO: 71 - DNA, 18 bp, Homo sapiens
  source 1..18: mol_type = other DNA
gtgatagatcttgactat

SEQ ID NO: 72 - protein, 6 aa, Homo sapiens
VIDLDY

SEQ ID NO: 73 - DNA, 321 bp, Homo sapiens
  source 1..321: mol_type = other DNA
gacatccagatgacccagtctccttccaccctgtctgcatctgaaggagacagagtcaccatcacttgccgggccagtcagagtattagtagctggttggcctggtatcaacagaaaccaggaaaagcccctaggctcctgatctataaggcgtctattttaggagatggggtcccatcaaggttcagcggcagtggatctgggacagaattcactctcaccatcagcagcctgcagcctgatgattttgctacttattactgccaccagtataatagttatttgtggacgttcggccaagggaccaaggtggaaatcaaa

SEQ ID NO: 74 - protein, 107 aa, Homo sapiens
DIQMTQSPSTLSASEGDRVTITCRASQSISSWLAWYQQKPGKAPRLLIYKASILGDGVPSRFSGSGSGTEFTLTISSLQPDDFATYYCHQYNSYLWTFGQGTKVEIK

SEQ ID NO: 75 - DNA, 18 bp, Homo sapiens
  source 1..18: mol_type = other DNA
cagagtattagtagctgg

SEQ ID NO: 76 - protein, 6 aa, Homo sapiens
QSISSW

SEQ ID NO: 77-78 - skipped (2 entries)

SEQ ID NO: 79 - DNA, 27 bp, Homo sapiens
  source 1..27: mol_type = other DNA
caccagtataatagttatttgtggacg

SEQ ID NO: 80 - protein, 9 aa, Homo sapiens
HQYNSYLWT

SEQ ID NO: 81 - DNA, 363 bp, Homo sapiens
  source 1..363: mol_type = other DNA
caggtgcagctgcaggagtcgggcccaggactggtgaagccttcacagaccctgtccctcacctgcactgtctctggtggctccatcagcagtgctgattactattggagctggatccgccagcacccagggaagggcctggagtggattggatccatctattatactgggagtacttactacaacccgtccctcaagagtcgacttaccatatcaatagacacgtctgagaaccagttctctttgaaactgacctctctgactgccgcggacacggccgtgtattactgtgcgagcgaggaggctaactggggatcccactttgactcctggggccagggaaccctggtcaccgtctcctca

SEQ ID NO: 82 - protein, 121 aa, Homo sapiens
QVQLQESGPGLVKPSQTLSLTCTVSGGSISSADYYWSWIRQHPGKGLEWIGSIYYTGSTYYNPSLKSRLTISIDTSENQFSLKLTSLTAADTAVYYCASEEANWGSHFDSWGQGTLVTVSS

SEQ ID NO: 83 - DNA, 30 bp, Homo sapiens
  source 1..30: mol_type = other DNA
ggtggctccatcagcagtgctgattactat

SEQ ID NO: 84 - protein, 10 aa, Homo sapiens
GGSISSADYY

SEQ ID NO: 85 - DNA, 21 bp, Homo sapiens
  source 1..21: mol_type = other DNA
atctattatactgggagtact

SEQ ID NO: 86 - protein, 7 aa, Homo sapiens
IYYTGST

SEQ ID NO: 87 - DNA, 39 bp, Homo sapiens
  source 1..39: mol_type = other DNA
gcgagcgaggaggctaactggggatcccactttgactcc

SEQ ID NO: 88 - protein, 13 aa, Homo sapiens
ASEEANWGSHFDS

SEQ ID NO: 89 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattgacaactttttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcatcttactactgtcaacatagtcacagtgcccatccgatcaccttcggccaagggacacgactggagattaaa

SEQ ID NO: 90 - protein, 108 aa, Homo sapiens
DIQMTQSPSSLSASVGDRVTITCRASQSIDNFLNWYQQKPGKAPKLLIYAASSLQSGVPSRFSGSGSGTDFTLTISSLQPEDFASYYCQHSHSAHPITFGQGTRLEIK

SEQ ID NO: 91 - DNA, 18 bp, Homo sapiens
  source 1..18: mol_type = other DNA
cagagcattgacaacttt

SEQ ID NO: 92 - protein, 6 aa, Homo sapiens
QSIDNF

SEQ ID NO: 93-94 - skipped (2 entries)

SEQ ID NO: 95 - DNA, 30 bp, Homo sapiens
  source 1..30: mol_type = other DNA
caacatagtcacagtgcccatccgatcacc

SEQ ID NO: 96 - protein, 10 aa, Homo sapiens
QHSHSAHPIT

SEQ ID NO: 97 - DNA, 363 bp, Homo sapiens
  source 1..363: mol_type = other DNA
cagctgcagctgcaggagtcgggcccaggactggtgaagccttcggagaccctgtccctcacctgcactgtctctggtggctccatcagcagtagtaattactactggggctggatccgccagcccccagggaagagactggagtggattgggagtatctattatagtgggagcacctactacaacccgtccctcaagactcgagtcaccatatccgtagacacgtccaagaatcagttctccctgaagctgacctctgtgaccgccgcagacacggctgtgtattactgtgcgagagaggaagcagcagctttgacgcactttgacttctggggccagggaaccctggtcaccgtctcctca

SEQ ID NO: 98 - protein, 121 aa, Homo sapiens
QLQLQESGPGLVKPSETLSLTCTVSGGSISSSNYYWGWIRQPPGKRLEWIGSIYYSGSTYYNPSLKTRVTISVDTSKNQFSLKLTSVTAADTAVYYCAREEAAALTHFDFWGQGTLVTVSS

SEQ ID NO: 99 - DNA, 30 bp, Homo sapiens
  source 1..30: mol_type = other DNA
ggtggctccatcagcagtagtaattactac

SEQ ID NO: 100 - protein, 10 aa, Homo sapiens
GGSISSSNYY

SEQ ID NO: 101 - DNA, 21 bp, Homo sapiens
  source 1..21: mol_type = other DNA
atctattatagtgggagcacc

SEQ ID NO: 102 - protein, 7 aa, Homo sapiens
IYYSGST

SEQ ID NO: 103 - DNA, 39 bp, Homo sapiens
  source 1..39: mol_type = other DNA
gcgagagaggaagcagcagctttgacgcactttgacttc

SEQ ID NO: 104 - protein, 13 aa, Homo sapiens
AREEAAALTHFDF

SEQ ID NO: 105 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcaactatttaaattggtatcagcagaaaccagggaaagcccctaagctcctgatctttgctgcatccagtttacaaagtggggtcccatcaaggttcagtggcagtggatctgggacagatttcactctcaccatcagcagtctgcaacctgaagattttgcaacttactactgtcaacatagtcacagttcccatccgatcaccttcggccaagggacacgactggagattaaa

SEQ ID NO: 106 - protein, 108 aa, Homo sapiens
DIQMTQSPSSLSASVGDRVTITCRASQSISNYLNWYQQKPGKAPKLLIFAASSLQSGVPSRFSGSGSGTDFTLTISSLQPEDFATYYCQHSHSSHPITFGQGTRLEIK

SEQ ID NO: 107 - DNA, 18 bp, Homo sapiens
  source 1..18: mol_type = other DNA
cagagcattagcaactat

SEQ ID NO: 108 - protein, 6 aa, Homo sapiens
QSISNY

SEQ ID NO: 109-110 - skipped (2 entries)

SEQ ID NO: 111 - DNA, 30 bp, Homo sapiens
  source 1..30: mol_type = other DNA
caacatagtcacagttcccatccgatcacc

SEQ ID NO: 112 - protein, 10 aa, Homo sapiens
QHSHSSHPIT

SEQ ID NO: 113 - DNA, 366 bp, Homo sapiens
  source 1..366: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattgggctggttataacatagactatgcggactctgtgaagggccgattcaccatctccagagacaacgccaagaactccctgtatctgcaaatgaacagtctgagagctgaggacacggccttgtattactgtgcaaaagatatgcgtggattcagttatggtttcccctttgactactggggccagggaaccctggtcaccgtctcctca

SEQ ID NO: 114 - protein, 122 aa, Homo sapiens
EVQLVESGGGLVQPGRSLRLSCAASGFTFDDYAMHWVRQAPGKGLEWVSGINWAGYNIDYADSVKGRFTISRDNAKNSLYLQMNSLRAEDTALYYCAKDMRGFSYGFPFDYWGQGTLVTVSS

SEQ ID NO: 115 - DNA, 24 bp, Homo sapiens
  source 1..24: mol_type = other DNA
ggattcacctttgatgattatgcc

SEQ ID NO: 116 - protein, 8 aa, Homo sapiens
GFTFDDYA

SEQ ID NO: 117 - DNA, 24 bp, Homo sapiens
  source 1..24: mol_type = other DNA
attaattgggctggttataacata

SEQ ID NO: 118 - protein, 8 aa, Homo sapiens
INWAGYNI

SEQ ID NO: 119 - DNA, 45 bp, Homo sapiens
  source 1..45: mol_type = other DNA
gcaaaagatatgcgtggattcagttatggtttcccctttgactac

SEQ ID NO: 120 - protein, 15 aa, Homo sapiens
AKDMRGFSYGFPFDY

SEQ ID NO: 121 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 122 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 123 - DNA, 18 bp, Homo sapiens
  source 1..18: mol_type = other DNA
cagagcattagcagctat

SEQ ID NO: 124 - protein, 6 aa, Homo sapiens
QSISSY

SEQ ID NO: 125-126 - skipped (2 entries)

SEQ ID NO: 127 - DNA, 30 bp, Homo sapiens
  source 1..30: mol_type = other DNA
caacagagttacagtacccctccgatcacc

SEQ ID NO: 128 - protein, 10 aa, Homo sapiens
QQSYSTPPIT

SEQ ID NO: 129 - DNA, 399 bp, Homo sapiens
  source 1..399: mol_type = other DNA
gaggtgcagctggtggagtctgggggaggcttggtaaagccgggggggtcccttagactctcctgtgcagcctctggatttattttcagtaacgcctggatgaactgggtccgccaggctccagggaagggactggcgtgggttggccgtattaaaaccgaaactgatggtgggacaacagactacgctgcacccgtaaaaggcagattcaccatctcaagagatgactcaaaaaacacgctgtatctgcaaatgaacagcgtgaaaaccgaggacacagccgtgtattactgtacagggggatacagctatggtgacgatagcagcagctggaacgagggctactactactacggtatggacgtctggggccaagggaccacggtcaccgtctcctca

SEQ ID NO: 130 - protein, 133 aa, Homo sapiens
EVQLVESGGGLVKPGGSLRLSCAASGFIFSNAWMNWVRQAPGKGLAWVGRIKTETDGGTTDYAAPVKGRFTISRDDSKNTLYLQMNSVKTEDTAVYYCTGGYSYGDDSSSWNEGYYYYGMDVWGQGTTVTVSS

SEQ ID NO: 131 - DNA, 24 bp, Homo sapiens
  source 1..24: mol_type = other DNA
ggatttattttcagtaacgcctgg

SEQ ID NO: 132 - protein, 8 aa, Homo sapiens
GFIFSNAW

SEQ ID NO: 133 - DNA, 30 bp, Homo sapiens
  source 1..30: mol_type = other DNA
attaaaaccgaaactgatggtgggacaaca

SEQ ID NO: 134 - protein, 10 aa, Homo sapiens
IKTETDGGTT

SEQ ID NO: 135 - DNA, 72 bp, Homo sapiens
  source 1..72: mol_type = other DNA
acagggggatacagctatggtgacgatagcagcagctggaacgagggctactactactacggtatggacgtc

SEQ ID NO: 136 - protein, 24 aa, Homo sapiens
TGGYSYGDDSSSWNEGYYYYGMDV

SEQ ID NO: 137 - DNA, 378 bp, Homo sapiens
  source 1..378: mol_type = other DNA
gaagtgcagctggtggagtctgggggaggcttggtacagcctggcaggtccctgagactctcctgtacagcctctggattcacctttgatgattatgccatgcactgggtccggcaagctccagggaagggcctggagtgggtctcaggtattcgttggaatggtggtagtataggctatgtggactctgtgaagggccgattcaccatctccagagacaacgccaagaagtccctgcatctgcaaatgaacagtctaaaaactgaggacacggccttgtattactgtgcaaaagatataggcgatattttgactggtttttatggagaatacggaatggacgtctggggccaagggaccacggtcaccgtctcctca

SEQ ID NO: 138 - protein, 126 aa, Homo sapiens
EVQLVESGGGLVQPGRSLRLSCTASGFTFDDYAMHWVRQAPGKGLEWVSGIRWNGGSIGYVDSVKGRFTISRDNAKKSLHLQMNSLKTEDTALYYCAKDIGDILTGFYGEYGMDVWGQGTTVTVSS

SEQ ID NO: 139 - DNA, 24 bp, Homo sapiens
  source 1..24: mol_type = other DNA
ggattcacctttgatgattatgcc

SEQ ID NO: 140 - protein, 8 aa, Homo sapiens
GFTFDDYA

SEQ ID NO: 141 - DNA, 24 bp, Homo sapiens
  source 1..24: mol_type = other DNA
attcgttggaatggtggtagtata

SEQ ID NO: 142 - protein, 8 aa, Homo sapiens
IRWNGGSI

SEQ ID NO: 143 - DNA, 57 bp, Homo sapiens
  source 1..57: mol_type = other DNA
gcaaaagatataggcgatattttgactggtttttatggagaatacggaatggacgtc

SEQ ID NO: 144 - protein, 19 aa, Homo sapiens
AKDIGDILTGFYGEYGMDV

SEQ ID NO: 145 - DNA, 321 bp, Homo sapiens
  source 1..321: mol_type = other DNA
gacatccagatgacccagtctccatcctccctgtctgcatctgaaggagacagagtcaccatcacttgccgggcaagtcagagcattagcagctatttaaattggtatcagcagaaagcagggaaagcccctaacctcctgatctatgctgcatccagtttgcaaagtggggtcccatcaaggttcagtggcagtggatctgggacagagtacactctcaccatcagcagtctgcaacctgaagattttgcaacttactactgtcaacagagttacattatcccgtacacttttggccaggggaccaagctggagatcaaa

SEQ ID NO: 146 - protein, 107 aa, Homo sapiens
DIQMTQSPSSLSASEGDRVTITCRASQSISSYLNWYQQKAGKAPNLLIYAASSLQSGVPSRFSGSGSGTEYTLTISSLQPEDFATYYCQQSYIIPYTFGQGTKLEIK

SEQ ID NO: 147 - DNA, 18 bp, Homo sapiens
  source 1..18: mol_type = other DNA
cagagcattagcagctat

SEQ ID NO: 148 - protein, 6 aa, Homo sapiens
QSISSY

SEQ ID NO: 149-150 - skipped (2 entries)

SEQ ID NO: 151 - DNA, 27 bp, Homo sapiens
  source 1..27: mol_type = other DNA
caacagagttacattatcccgtacact

SEQ ID NO: 152 - protein, 9 aa, Homo sapiens
QQSYIIPYT

SEQ ID NO: 153 - DNA, 378 bp, Homo sapiens
  source 1..378: mol_type = other DNA
gaagtgcagctggtggagtctgggggagggttggtacagcctggcaggtccctgagactctcctgtgcagcctctggattcacctttgatgattatgccatgcactgggtccggcaagctccagggaagggcctggagtgggtctcaagtgttaggtggaatggtggtattataggctatgcggactctgtgaagggccgattcaccatctccagagacaacgccaagaactccctgtatctgcaaatgaacagtctgagacctgaggacacggccctctattactgtgcaaaagatataggcgatgttttgactggttattatggagaatacggtatggacgtctggggccaagggaccacggtcaccgtctcctca

SEQ ID NO: 154 - protein, 126 aa, Homo sapiens
EVQLVESGGGLVQPGRSLRLSCAASGFTFDDYAMHWVRQAPGKGLEWVSSVRWNGGIIGYADSVKGRFTISRDNAKNSLYLQMNSLRPEDTALYYCAKDIGDVLTGYYGEYGMDVWGQGTTVTVSS

SEQ ID NO: 155 - DNA, 24 bp, Homo sapiens
  source 1..24: mol_type = other DNA
ggattcacctttgatgattatgcc

SEQ ID NO: 156 - protein, 8 aa, Homo sapiens
GFTFDDYA

SEQ ID NO: 157 - DNA, 24 bp, Homo sapiens
  source 1..24: mol_type = other DNA
gttaggtggaatggtggtattata

SEQ ID NO: 158 - protein, 8 aa, Homo sapiens
VRWNGGII

SEQ ID NO: 159 - DNA, 57 bp, Homo sapiens
  source 1..57: mol_type = other DNA
gcaaaagatataggcgatgttttgactggttattatggagaatacggtatggacgtc

SEQ ID NO: 160 - protein, 19 aa, Homo sapiens
AKDIGDVLTGYYGEYGMDV

SEQ ID NO: 161 - DNA, 321 bp, Homo sapiens
  source 1..321: mol_type = other DNA
gacatccagatgacccagtctccatcctccctgtctgcatctgtgggagacagagtcaccatcgcttgccgggcaagtcagagcattaccacctatttaaattggtatcagcagaaaccagggaaagcccctaaactcctgatctatgctgcatccagtttgcaaagtggggtcccatcaaggttcagtggcagtggatctgggacagatttcactctcaccatcagtagtctgcaacctgaagattttgcaacttactactgtcaacagagttacatttccccgtacacttttggccaggggaccaagctggagatcaaa

SEQ ID NO: 162 - protein, 107 aa, Homo sapiens
DIQMTQSPSSLSASVGDRVTIACRASQSITTYLNWYQQKPGKAPKLLIYAASSLQSGVPSRFSGSGSGTDFTLTISSLQPEDFATYYCQQSYISPYTFGQGTKLEIK

SEQ ID NO: 163 - DNA, 18 bp, Homo sapiens
  source 1..18: mol_type = other DNA
cagagcattaccacctat

SEQ ID NO: 164 - protein, 6 aa, Homo sapiens
QSITTY

SEQ ID NO: 165-166 - skipped (2 entries)

SEQ ID NO: 167 - DNA, 27 bp, Homo sapiens
  source 1..27: mol_type = other DNA
caacagagttacatttccccgtacact

SEQ ID NO: 168 - protein, 9 aa, Homo sapiens
QQSYISPYT

SEQ ID NO: 169 - DNA, 378 bp, Homo sapiens
  source 1..378: mol_type = other DNA
caggtgcagctggtggagtctgggggaggcgtggtccagcctgggaagtccctgagactctcctgtgcagcctctggattcaccttcagtaattatggcatacactgggtccgccaggctccaggcaaggggctggagtgggtggcgattatattatatgatggaagtaatcaacactatgcagattccgtgaagggccgattcaccatttccagagacaattccaaaaacacgctgtatcttcaaatgaacaacctgagagctgaggacacggccgtttattactgtgcgagagatcttgatctttggagtggttattatacaaacggggacggtatggacgtctggggccaagggaccacggtcaccgtctcctca

SEQ ID NO: 170 - protein, 126 aa, Homo sapiens
QVQLVESGGGVVQPGKSLRLSCAASGFTFSNYGIHWVRQAPGKGLEWVAIILYDGSNQHYADSVKGRFTISRDNSKNTLYLQMNNLRAEDTAVYYCARDLDLWSGYYTNGDGMDVWGQGTTVTVSS

SEQ ID NO: 171 - DNA, 24 bp, Homo sapiens
  source 1..24: mol_type = other DNA
ggattcaccttcagtaattatggc

SEQ ID NO: 172 - protein, 8 aa, Homo sapiens
GFTFSNYG

SEQ ID NO: 173 - DNA, 24 bp, Homo sapiens
  source 1..24: mol_type = other DNA
atattatatgatggaagtaatcaa

SEQ ID NO: 174 - protein, 8 aa, Homo sapiens
ILYDGSNQ

SEQ ID NO: 175 - DNA, 57 bp, Homo sapiens
  source 1..57: mol_type = other DNA
gcgagagatcttgatctttggagtggttattatacaaacggggacggtatggacgtc

SEQ ID NO: 176 - protein, 19 aa, Homo sapiens
ARDLDLWSGYYTNGDGMDV

SEQ ID NO: 177 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 178 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 179 - DNA, 1338 bp, Homo sapiens
  source 1..1338: mol_type = other DNA
gaagtgcagctggtggagtctgggggaggcttggtgcagcctggcaggtccctgagactctcctgtgcggcctctggattcacctttgatgattatgccatacactgggtccggcaagctccagggaagggcctggagtgggtctcagttatcagttggaatagtgatatcataggctatgcggactctgtgaagggccgattcaccgtctccagagacaacgccaagaactccctgtatctgcaaatgaatagtctgagaactgaggacacggccttgtattactgtgcaaaaggatataactggaacttctttgactattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 180 - protein, 445 aa, Homo sapiens
EVQLVESGGGLVQPGRSLRLSCAASGFTFDDYAIHWVRQAPGKGLEWVSVISWNSDIIGYADSVKGRFTVSRDNAKNSLYLQMNSLRTEDTALYYCAKGYNWNFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 181 - DNA, 642 bp, Homo sapiens
  source 1..642: mol_type = other DNA
gaaattgtgttgacgcagtctccagccaccctgtctttatctccaggggaaagagccaccctctcctgcagggccagtcagagtgttagcagctacttagcctggtaccaacagaaacctggccaggctcccaggctcctcatctataatgcagcaaacagggccactgacatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 182 - protein, 214 aa, Homo sapiens
EIVLTQSPATLSLSPGERATLSCRASQSVSSYLAWYQQKPGQAPRLLIYNAANRATDIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 1338 bp, Homo sapiens
  source 1..1338: mol_type = other DNA
gaagtgcagctggtggagtctgggggaggcttggtacagcctggcaggtccctgagactctcctgtgcagcctctggattcacctttgatgattatgccatgcactgggtccggcaaactccagggaagggcctggagtgggtctcagttattagttggaatagtgatgtcatagcctattcggactctgtgaagggccgcttcaccatttccagagacaacgccaagaactccctgtatctgcaaatgaacagtctgggaactgaggacacggccttatattactgtgcaaaaggccataactggaacttctttgactattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 184 - protein, 445 aa, Homo sapiens
EVQLVESGGGLVQPGRSLRLSCAASGFTFDDYAMHWVRQTPGKGLEWVSVISWNSDVIAYSDSVKGRFTISRDNAKNSLYLQMNSLGTEDTALYYCAKGHNWNFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - DNA, 642 bp, Homo sapiens
  source 1..642: mol_type = other DNA
gaaattgtgttgacacagtctccagccaccctgtctttgtctccaggagaaagagccaccctctcctgcagggccagtcagagtgttagcagctacttagcctggtaccaacagaaacctggccaggctcccaggctcctcatctataatgtagccaacagggccacagacatcccagccaggttcagtggcagtgggtctgggacagacttcactctcaccatcagcggcctagagcctgaagattttgcagtttatttctgtcagcagcgtagcaactggcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 186 - protein, 214 aa, Homo sapiens
EIVLTQSPATLSLSPGERATLSCRASQSVSSYLAWYQQKPGQAPRLLIYNVANRATDIPARFSGSGSGTDFTLTISGLEPEDFAVYFCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - DNA, 1338 bp, Homo sapiens
  source 1..1338: mol_type = other DNA
gaagtgcagctggtggagtctgggggaggcttggtacagcctggcaggtccctgagactctcctgtacagcctctggattcacctttgatgattatgccatacactgggtccggcaatctccagggaagggcctggagtgggtctcagttatcagttggaatagtgatgtcataggctatgcggactctgtgaagggccgattcaccatctccagagacaacgccaagaactccctgtatctgcagatgaatagtctgagagctgaggacacggccttgtattactgtgcaaaaggatataactggaacttctttgactattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 188 - protein, 445 aa, Homo sapiens
EVQLVESGGGLVQPGRSLRLSCTASGFTFDDYAIHWVRQSPGKGLEWVSVISWNSDVIGYADSVKGRFTISRDNAKNSLYLQMNSLRAEDTALYYCAKGYNWNFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 189 - DNA, 642 bp, Homo sapiens; identical to SEQ ID NO: 181
  source 1..642: mol_type = other DNA

SEQ ID NO: 190 - protein, 214 aa, Homo sapiens; identical to SEQ ID NO: 182

SEQ ID NO: 191 - DNA, 1338 bp, Homo sapiens
  source 1..1338: mol_type = other DNA
gaagtgcagctggtggagtctgggggagacttggtacagcctggcaggtccctgagactctcctgtgcagcctctggattcacctttgatgattatgccatgcactgggtccggcaagctccagggaagggcctggaatgggtctcagttattagttggaatagtgatgtcatagcctattcggactctgtgaagggccgattcaccatctccagagacaacgccaagaactccctgtatctgcaaatgaacagtctgagaactgaggacacggccttatattactgtacaaaaggccataagtggagcttctttgactattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 192 - protein, 445 aa, Homo sapiens
EVQLVESGGDLVQPGRSLRLSCAASGFTFDDYAMHWVRQAPGKGLEWVSVISWNSDVIAYSDSVKGRFTISRDNAKNSLYLQMNSLRTEDTALYYCTKGHKWSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 193 - DNA, 642 bp, Homo sapiens
  source 1..642: mol_type = other DNA
gaaattgtgttgacacagtctccagccaccctgtctttgtctccaggggaaagagccaccctctcctgcagggccagtcagagtattagcagctacttagcctggtaccaacagaaacctggccaggctcccagactcctcatctttaatgtagccaacagggccactgacatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 194 - protein, 214 aa, Homo sapiens
EIVLTQSPATLSLSPGERATLSCRASQSISSYLAWYQQKPGQAPRLLIFNVANRATDIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - DNA, 1323 bp, Homo sapiens
  source 1..1323: mol_type = other DNA
gaggtgcagctggtggagtctgggggaggcttggttcagcctggggggtccctgagactctcctgcgcagcctctggattcacctttagcgactatgccatgagctgggtccgccaggctccggggaaggggctggagtgggtctcaggtattagtggaaatggtggtgacacatactacggagacttcgtgaagggccggttcaccatctccagagacaattccaagaacacgctgtatctgcaaatgaacagcctgagaggcgaggacacggccgcatatttctgtgtgatagatcttgactattggggtcagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 196 - protein, 440 aa, Homo sapiens
EVQLVESGGGLVQPGGSLRLSCAASGFTFSDYAMSWVRQAPGKGLEWVSGISGNGGDTYYGDFVKGRFTISRDNSKNTLYLQMNSLRGEDTAAYFCVIDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 197 - DNA, 642 bp, Homo sapiens
  source 1..642: mol_type = other DNA
gacatccagatgacccagtctccttccaccctgtctgcatctgaaggagacagagtcaccatcacttgccgggccagtcagagtattagtagctggttggcctggtatcaacagaaaccaggaaaagcccctaggctcctgatctataaggcgtctattttaggagatggggtcccatcaaggttcagcggcagtggatctgggacagaattcactctcaccatcagcagcctgcagcctgatgattttgctacttattactgccaccagtataatagttatttgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 198 - protein, 214 aa, Homo sapiens
DIQMTQSPSTLSASEGDRVTITCRASQSISSWLAWYQQKPGKAPRLLIYKASILGDGVPSRFSGSGSGTEFTLTISSLQPDDFATYYCHQYNSYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - DNA, 1347 bp, Homo sapiens
  source 1..1347: mol_type = other DNA
caggtgcagctgcaggagtcgggcccaggactggtgaagccttcacagaccctgtccctcacctgcactgtctctggtggctccatcagcagtgctgattactattggagctggatccgccagcacccagggaagggcctggagtggattggatccatctattatactgggagtacttactacaacccgtccctcaagagtcgacttaccatatcaatagacacgtctgagaaccagttctctttgaaactgacctctctgactgccgcggacacggccgtgtattactgtgcgagcgaggaggctaactggggatcccactttgactcctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 200 - protein, 448 aa, Homo sapiens
QVQLQESGPGLVKPSQTLSLTCTVSGGSISSADYYWSWIRQHPGKGLEWIGSIYYTGSTYYNPSLKSRLTISIDTSENQFSLKLTSLTAADTAVYYCASEEANWGSHFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 201 - DNA, 645 bp, Homo sapiens
  source 1..645: mol_type = other DNA
gacatccagatgacccagtctccatcctccctgtctgcatctgtaggagacagagtcaccatcacttgccgggcaagtcagagcattgacaactttttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcatcttactactgtcaacatagtcacagtgcccatccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 202 - protein, 215 aa, Homo sapiens
DIQMTQSPSSLSASVGDRVTITCRASQSIDNFLNWYQQKPGKAPKLLIYAASSLQSGVPSRFSGSGSGTDFTLTISSLQPEDFASYYCQHSHSAHPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - DNA, 1347 bp, Homo sapiens
  source 1..1347: mol_type = other DNA
cagctgcagctgcaggagtcgggcccaggactggtgaagccttcggagaccctgtccctcacctgcactgtctctggtggctccatcagcagtagtaattactactggggctggatccgccagcccccagggaagagactggagtggattgggagtatctattatagtgggagcacctactacaacccgtccctcaagactcgagtcaccatatccgtagacacgtccaagaatcagttctccctgaagctgacctctgtgaccgccgcagacacggctgtgtattactgtgcgagagaggaagcagcagctttgacgcactttgacttctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 204 - protein, 448 aa, Homo sapiens
QLQLQESGPGLVKPSETLSLTCTVSGGSISSSNYYWGWIRQPPGKRLEWIGSIYYSGSTYYNPSLKTRVTISVDTSKNQFSLKLTSVTAADTAVYYCAREEAAALTHFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 205 - DNA, 645 bp, Homo sapiens
  source 1..645: mol_type = other DNA
gacatccagatgacccagtctccatcctccctgtctgcatctgtaggagacagagtcaccatcacttgccgggcaagtcagagcattagcaactatttaaattggtatcagcagaaaccagggaaagcccctaagctcctgatctttgctgcatccagtttacaaagtggggtcccatcaaggttcagtggcagtggatctgggacagatttcactctcaccatcagcagtctgcaacctgaagattttgcaacttactactgtcaacatagtcacagttcccatccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 206 - protein, 215 aa, Homo sapiens
DIQMTQSPSSLSASVGDRVTITCRASQSISNYLNWYQQKPGKAPKLLIFAASSLQSGVPSRFSGSGSGTDFTLTISSLQPEDFATYYCQHSHSSHPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 1350 bp, Homo sapiens
  source 1..1350: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattgggctggttataacatagactatgcggactctgtgaagggccgattcaccatctccagagacaacgccaagaactccctgtatctgcaaatgaacagtctgagagctgaggacacggccttgtattactgtgcaaaagatatgcgtggattcagttatggtttcccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 208 - protein, 449 aa, Homo sapiens
EVQLVESGGGLVQPGRSLRLSCAASGFTFDDYAMHWVRQAPGKGLEWVSGINWAGYNIDYADSVKGRFTISRDNAKNSLYLQMNSLRAEDTALYYCAKDMRGFSYGFPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 209 - DNA, 645 bp, Homo sapiens
  source 1..645: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 210 - protein, 214 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - DNA, 1383 bp, Homo sapiens
  source 1..1383: mol_type = other DNA
gaggtgcagctggtggagtctgggggaggcttggtaaagccgggggggtcccttagactctcctgtgcagcctctggatttattttcagtaacgcctggatgaactgggtccgccaggctccagggaagggactggcgtgggttggccgtattaaaaccgaaactgatggtgggacaacagactacgctgcacccgtaaaaggcagattcaccatctcaagagatgactcaaaaaacacgctgtatctgcaaatgaacagcgtgaaaaccgaggacacagccgtgtattactgtacagggggatacagctatggtgacgatagcagcagctggaacgagggctactactactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 212 - protein, 460 aa, Homo sapiens
EVQLVESGGGLVKPGGSLRLSCAASGFIFSNAWMNWVRQAPGKGLAWVGRIKTETDGGTTDYAAPVKGRFTISRDDSKNTLYLQMNSVKTEDTAVYYCTGGYSYGDDSSSWNEGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - DNA, 645 bp, Homo sapiens; identical to SEQ ID NO: 209
  source 1..645: mol_type = other DNA

SEQ ID NO: 214 - protein, 214 aa, Homo sapiens; identical to SEQ ID NO: 210

SEQ ID NO: 215 - DNA, 1362 bp, Homo sapiens
  source 1..1362: mol_type = other DNA
gaagtgcagctggtggagtctgggggaggcttggtacagcctggcaggtccctgagactctcctgtacagcctctggattcacctttgatgattatgccatgcactgggtccggcaagctccagggaagggcctggagtgggtctcaggtattcgttggaatggtggtagtataggctatgtggactctgtgaagggccgattcaccatctccagagacaacgccaagaagtccctgcatctgcaaatgaacagtctaaaaactgaggacacggccttgtattactgtgcaaaagatataggcgatattttgactggtttttatggagaatacggaatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 216 - protein, 453 aa, Homo sapiens
EVQLVESGGGLVQPGRSLRLSCTASGFTFDDYAMHWVRQAPGKGLEWVSGIRWNGGSIGYVDSVKGRFTISRDNAKKSLHLQMNSLKTEDTALYYCAKDIGDILTGFYGEYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 217 - DNA, 642 bp, Homo sapiens
  source 1..642: mol_type = other DNA
gacatccagatgacccagtctccatcctccctgtctgcatctgaaggagacagagtcaccatcacttgccgggcaagtcagagcattagcagctatttaaattggtatcagcagaaagcagggaaagcccctaacctcctgatctatgctgcatccagtttgcaaagtggggtcccatcaaggttcagtggcagtggatctgggacagagtacactctcaccatcagcagtctgcaacctgaagattttgcaacttactactgtcaacagagttacattatcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 218 - protein, 214 aa, Homo sapiens
DIQMTQSPSSLSASEGDRVTITCRASQSISSYLNWYQQKAGKAPNLLIYAASSLQSGVPSRFSGSGSGTEYTLTISSLQPEDFATYYCQQSYIIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - DNA, 1362 bp, Homo sapiens
  source 1..1362: mol_type = other DNA
gaagtgcagctggtggagtctgggggagggttggtacagcctggcaggtccctgagactctcctgtgcagcctctggattcacctttgatgattatgccatgcactgggtccggcaagctccagggaagggcctggagtgggtctcaagtgttaggtggaatggtggtattataggctatgcggactctgtgaagggccgattcaccatctccagagacaacgccaagaactccctgtatctgcaaatgaacagtctgagacctgaggacacggccctctattactgtgcaaaagatataggcgatgttttgactggttattatggagaatacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 220 - protein, 453 aa, Homo sapiens
EVQLVESGGGLVQPGRSLRLSCAASGFTFDDYAMHWVRQAPGKGLEWVSSVRWNGGIIGYADSVKGRFTISRDNAKNSLYLQMNSLRPEDTALYYCAKDIGDVLTGYYGEYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 221 - DNA, 642 bp, Homo sapiens
  source 1..642: mol_type = other DNA
gacatccagatgacccagtctccatcctccctgtctgcatctgtgggagacagagtcaccatcgcttgccgggcaagtcagagcattaccacctatttaaattggtatcagcagaaaccagggaaagcccctaaactcctgatctatgctgcatccagtttgcaaagtggggtcccatcaaggttcagtggcagtggatctgggacagatttcactctcaccatcagtagtctgcaacctgaagattttgcaacttactactgtcaacagagttacatttccccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 222 - protein, 214 aa, Homo sapiens
DIQMTQSPSSLSASVGDRVTIACRASQSITTYLNWYQQKPGKAPKLLIYAASSLQSGVPSRFSGSGSGTDFTLTISSLQPEDFATYYCQQSYISPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - DNA, 1362 bp, Homo sapiens
  source 1..1362: mol_type = other DNA
caggtgcagctggtggagtctgggggaggcgtggtccagcctgggaagtccctgagactctcctgtgcagcctctggattcaccttcagtaattatggcatacactgggtccgccaggctccaggcaaggggctggagtgggtggcgattatattatatgatggaagtaatcaacactatgcagattccgtgaagggccgattcaccatttccagagacaattccaaaaacacgctgtatcttcaaatgaacaacctgagagctgaggacacggccgtttattactgtgcgagagatcttgatctttggagtggttattatacaaacggggacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 224 - protein, 453 aa, Homo sapiens
QVQLVESGGGVVQPGKSLRLSCAASGFTFSNYGIHWVRQAPGKGLEWVAIILYDGSNQHYADSVKGRFTISRDNSKNTLYLQMNNLRAEDTAVYYCARDLDLWSGYYTNGDGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 225 - DNA, 645 bp, Homo sapiens; identical to SEQ ID NO: 209
  source 1..645: mol_type = other DNA

SEQ ID NO: 226 - protein, 214 aa, Homo sapiens; identical to SEQ ID NO: 210

SEQ ID NO: 227 - protein, 346 aa, synthetic construct
  REGION 1..346: note = Recombinant Human IL-36R polypeptide (hIL-36R.mmH)
DGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYEQKLISEEDLGGEQKLISEEDLHHHHHH

SEQ ID NO: 228 - protein, 7 aa, synthetic construct
  REGION 1..7: note = Synthetic peptide
YKQILHL

SEQ ID NO: 229 - protein, 10 aa, synthetic construct
  REGION 1..10: note = Synthetic peptide
YKQILHLGKD

SEQ ID NO: 230 - protein, 14 aa, synthetic construct
  REGION 1..14: note = Synthetic peptide
GVETHVSFREHNYL

SEQ ID NO: 231 - protein, 4 aa, synthetic construct
  REGION 1..4: note = Synthetic peptide
ILHL

SEQ ID NO: 232 - protein, 7 aa, synthetic construct
  REGION 1..7: note = Synthetic peptide
ILHLGKD

SEQ ID NO: 233 - protein, 8 aa, synthetic construct
  REGION 1..8: note = Synthetic peptide
GVETHVSF

SEQ ID NO: 234 - protein, 5 aa, synthetic construct
  REGION 1..5: note = Synthetic peptide
THVSF

SEQ ID NO: 235 - protein, 4 aa, synthetic construct
  REGION 1..4: note = Synthetic peptide
HVSF

SEQ ID NO: 236 - protein, 9 aa, synthetic construct
  REGION 1..9: note = Synthetic peptide
HVSFREHNL

SEQ ID NO: 237 - protein, 10 aa, synthetic construct
  REGION 1..10: note = Synthetic peptide
HVSFREHNLY

SEQ ID NO: 238 - protein, 6 aa, synthetic construct
  REGION 1..6: note = Synthetic peptide
FREHNL

SEQ ID NO: 239 - protein, 446 aa, synthetic construct
  REGION 1..446: note = Polypeptide: APE6155 heavy chain (comprising an IgG4 constant domain)
QVQLVQSGAEVKKPGASVKVSCKASGYTFTNYWMNWVRQAPRQGLEWMGMFHPTGDVTRLNQKFKDRVTMTRDTSTSTVYMELSSLRSEDTAVYYCARTTSMIIGGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 240 - protein, 219 aa, synthetic construct
  REGION 1..219: note = Polypeptide: APE6155 light chain (comprising a Kappa constant domain)
DIVMTQTPLSLSVTPGQPASISCRSSKSLLHRNAITYFYWYLHKPGQPPQLLIYQMSNLASGVPDRFSGSGSGTDFTLKISRVEAEDVGVYYCAQNLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 119 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDYNWNYYFDYWGQGTLVTVSS

SEQ ID NO: 242 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 243 - protein, 120 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDYSGSYYYFDYWGQGTLVTVSS

(19) United States
(12) Patent Application Publication
(10) Pub. No.: US 2025/0011442 A1
(43) Pub. Date: Jan. 9, 2025

(54) ANTI-IL36R ANTIBODIES

(71) Applicant: Regeneron Pharmaceuticals, Inc., Tarrytown, NY (US)

(72) Inventors: Jeanette FAIRHURST, Thornwood, NY (US); Elena GARNOVA, White Plains, NY (US); William OLSON, Yorktown Heights, NY (US); Sokol HAXHINASTO, Brookfield, CT (US)

(73) Assignee: Regeneron Pharmaceuticals, Inc., Tarrytown, NY (US)

(21) Appl. No.: 18/760,617

(22) Filed: Jul. 1, 2024

Related U.S. Application Data
(63) Continuation of application No. 17/590,319, filed on Feb. 1, 2022, now Pat. No. 12,060,428.
Application No. 17/590,319 is a division of application No. 16/512,886, filed on Jul. 16, 2019, now Pat. No. 11,267,893.
(60) Provisional application No. 62/866,028, filed on Jun. 25, 2019.
(60) Provisional application No. 62/846,989, filed on May 13, 2019.
(60) Provisional application No. 62/698,482, filed on Jul. 16, 2018.

(51) Int. Cl. C07K 16/28; A61K 9/00; A61K 45/06; A61P 1/00; A61P 17/00

(52) U.S. Cl. CPC C07K 16/2866; A61K 9/0019; A61P 1/00; A61P 17/00; A61K 45/06

(57) ABSTRACT

The present invention provides antibodies and antigen-binding fragments (e.g., human antibodies) that bind specifically to human Interleukin-36 receptor (IL36R). Methods for treating or preventing diseases mediated by IL36R (e.g., skin or colon inflammatory conditions such as palmo-plantar pustular psoriasis, palmoplantar pustulosis, generalized pustular psoriasis, ulcerative colitis or IBD) using the antibodies and fragments are also provided along with methods of making the antibodies and fragments.

### CROSS REFERENCE TO RELATED APPLICATIONS

This application is a continuation of U.S. patent application Ser. No. 17/590,319, filed Feb. 1, 2022, which is a divisional of U.S. patent application Ser. No. 16/512,886, filed Jul. 16, 2019, now U.S. Pat. No. 11,267,893, which claims the benefit of U.S. Provisional Patent Application No. 62/698,482, filed Jul. 16, 2018; U.S. Provisional Patent Application No. 62/846,989, filed May 13, 2019; and U.S. Provisional Patent Application No. 62/866,028, filed Jun. 25, 2019; each of which is herein incorporated by reference in its entirety.

### FIELD OF THE INVENTION

The field of the invention relates, in part, to antibodies that bind to IL-36 receptor and the use of such antibodies to treat inflammatory disorders including psoriasis or inflammatory bowel disease.

### INCORPORATION BY REFERENCE OF SEQUENCE LISTING

The Sequence Listing in the XML file, named as 36432AZ_10484US03_SequenceListing.xml of 242 KB, created on Jun. 27, 2024 and submitted to the United States Patent and Trademark Office via Patent Center, is incorporated herein by reference.

### BACKGROUND OF THE INVENTION

The interleukin (IL)-36 cytokines include 3 agonists, IL-36α, IL-36β, and IL-36γ that bind to a common receptor composed of IL-36R and IL-1RAcP to stimulate inflammatory responses. IL-36 receptor (IL-36R) is a single-pass membrane receptor for a subset of the IL-1 family of cytokines, IL-36α, IL-36β, and IL-367, and upon binding to any of these ligands, there is recruitment of its co-receptor, the IL-1R accessory protein (IL-1RAcP), which induces a signaling cascade that involves NFκB and mitogen-activated kinase pathways (Sims et al, 2010.

A mediator of some inflammatory skin conditions, such as psoriasis, is IL-36. Psoriasis is a common, immune-mediated, inflammatory skin disease which includes the variants plaque psoriasis and generalized pustular psoriasis. Standard therapeutic guidelines include the use of topical steroids, topical vitamin D, systemic immunosuppressants and various biologics, such as anti-tumor necrosis factor (TNF) α, anti-interleukin (IL)-23 and anti-IL-17 antibodies. IL-36 members are overexpressed in the lesional skin of plaque psoriasis and activation of IL-36R might contribute to the persistence and perpetuation of psoriatic inflammation together with the TNF-α/IL-23/IL-17/IL-22 axis. (Di Cesare et al., The IL-23/Th17 axis in the immunopathogenesis of psoriasis, Journal of Investigative Dermatology 129: 1339-1350 (2009) and Blumberg et al., IL-1RL2 and its ligands contribute to the cytokine network in psoriasis. J Immunol 185: 4354-4362 (2010)).

Currently available treatments for palmoplantar pustulosis (PPP) and palmoplantar pustular psoriasis (PPPP), however, are limited. Spesolimab and ANB019 are anti-IL36R antibodies in clinical development which suffer from drawbacks related to immunogenicity and potency.

### SUMMARY OF THE INVENTION

The present invention provides anti-IL36R antibodies and antigen-binding fragments thereof that exhibit superior properties. For example, we observed in pharmacokinetic studies in three cynomolgous monkeys per group (0.5 and 5 mg/kg subcutaneous dose groups; n=3/group), that the anti-IL36R antibodies set forth herein (e.g., H4H14708P2) exhibited about 1.2-fold greater exposure than anti-IL36R antibody, APE6155. Moreover, we also observed that APE6155 exhibited less potency than anti-IL36R antibodies set forth herein, e.g., in reducing skin thickness and pathology scores in IMQ-induced skin inflammation and in reducing pro-inflammatory cytokines in skin. Spesolimab, a humanized anti-IL36R antibody, exhibited high levels of anti-drug antibody in human subjects with GPP. In a Phase 1 clinical trial, 3 of 7 patients had anti-drug antibodies at week 2 and these sustained to week 20 after a single dose. This property of spesolimab would not be ideal for chronic long-term treatment. Amin, First Data in GPP from Competitor Anti-IL36 Provides Proof of Concept of ANB019, Flash Note, Company Update, AnaptysBio, Jefferies (Sep. 16, 2018). The human anti-IL36R antibodies of the present invention are not expected to be highly immunogenic in humans.

The present invention provides an antigen-binding protein (e.g., an antibody or antigen-binding fragment thereof, e.g., a human antibody or antigen-binding fragment thereof or a multispecific antibody) that (i) specifically binds to the same epitope on IL36R as a reference antigen-binding protein; or (ii) competes for binding to IL36R polypeptide with a reference antigen-binding protein, wherein the reference antigen-binding protein comprises: (a) a heavy chain immunoglobulin that comprises CDR-H1, CDR-H2 and CDR-H3 of a heavy chain immunoglobulin that comprises the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and/or (b) a light chain immunoglobulin that comprises CDR-L1, CDR-L2 and CDR-L3 of a light chain immunoglobulin that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226. For example, in an embodiment of the invention, the antigen-binding protein comprises: (i) a heavy chain immunoglobulin that comprises CDR-H1, CDR-H2 and CDR-H3 of a heavy chain immunoglobulin that comprises the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and/or (ii) a light chain immunoglobulin that comprises CDR-L1, CDR-L2 and CDR-L3 of a light chain immunoglobulin that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226. In an embodiment of the invention, the antigen-binding protein comprises (a) a heavy chain immunoglobulin variable region comprising an amino acid sequence having at least 90% amino acid sequence identity to the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and/or (b) a light chain immunoglobulin variable region comprising an amino acid sequence having at least 90% amino acid sequence identity to the amino acid sequence set forth in SEQ ID NO: 10, 26, 42, 58, 74, 90, 106, 122, 146, 162, 182, 186, 190, 194, 198, 202, 206, 210, 214, 218, 222 or 226. For example, in an embodiment of the invention, the antigen-binding protein comprises: (a) a heavy chain immunoglobulin comprising the CDR-H1, CDR-H2 and CDR-H3 of a heavy chain immunoglobulin comprising an amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224 and at least 90% amino acid sequence identity to the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and/or (b) an light chain immunoglobulin comprising the CDR-L1, CDR-L2 and CDR-L3 of a light chain immunoglobulin comprising an amino acid sequence set forth in SEQ ID NO: 10, 26, 42, 58, 74, 90, 106, 122, 146, 162, 182, 186, 190, 194, 198, 202, 206, 210, 214, 218, 222 or 226 and at least 90% amino acid sequence identity to the amino acid sequence set forth in SEQ ID NO: 10, 26, 42, 58, 74, 90, 106, 122, 146, 162, 182, 186, 190, 194, 198, 202, 206, 210, 214, 218, 222 or 226. In an embodiment of the invention, the antigen-binding protein comprises: a heavy chain immunoglobulin that comprises: CDR-H1 comprising the amino acid sequence set forth in SEQ ID NO: 4; CDR-H2 comprising the amino acid sequence set forth in SEQ ID NO: 6; and CDR-H3 comprising the amino acid sequence set forth in SEQ ID NO: 8 and/or CDR-H1 comprising the amino acid sequence set forth in SEQ ID NO: 20; CDR-H2 comprising the amino acid sequence set forth in SEQ ID NO: 22; and CDR-H3 comprising the amino acid sequence set forth in SEQ ID NO: 24 and/or CDR-H1 comprising the amino acid sequence set forth in SEQ ID NO: 36; CDR-H2 comprising the amino acid sequence set forth in SEQ ID NO: 38; and CDR-H3 comprising the amino acid sequence set forth in SEQ ID NO: 40 and/or CDR-H1 comprising the amino acid sequence set forth in SEQ ID NO: 52; CDR-H2 comprising the amino acid sequence set forth in SEQ ID NO: 54; and CDR-H3 comprising the amino acid sequence set forth in SEQ ID NO: 56 and/or CDR-H1 comprising the amino acid sequence set forth in SEQ ID NO: 68; CDR-H2 comprising the amino acid sequence set forth in SEQ ID NO: 70; and CDR-H3 comprising the amino acid sequence set forth in SEQ ID NO: 72 and/or CDR-H1 comprising the amino acid sequence set forth in SEQ ID NO: 84; CDR-H2 comprising the amino acid sequence set forth in SEQ ID NO: 86; and CDR-H3 comprising the amino acid sequence set forth in SEQ ID NO: 88 and/or CDR-H1 comprising the amino acid sequence set forth in SEQ ID NO: 100; CDR-H2 comprising the amino acid sequence set forth in SEQ ID NO: 102; and CDR-H3 comprising the amino acid sequence set forth in SEQ ID NO: 104 and/or CDR-H1 comprising the amino acid sequence set forth in SEQ ID NO: 116; CDR-H2 comprising the amino acid sequence set forth in SEQ ID NO: 118; and CDR-H3 comprising the amino acid sequence set forth in SEQ ID NO: 120 and/or CDR-H1 comprising the amino acid sequence set forth in SEQ ID NO: 132; CDR-H2 comprising the amino acid sequence set forth in SEQ ID NO: 134; and CDR-H3 comprising the amino acid sequence set forth in SEQ ID NO: 136 and/or CDR-H1 comprising the amino acid sequence set forth in SEQ ID NO: 140; CDR-H2 comprising the amino acid sequence set forth in SEQ ID NO: 142; and CDR-H3 comprising the amino acid sequence set forth in SEQ ID NO: 144 and/or CDR-H1 comprising the amino acid sequence set forth in SEQ ID NO: 156; CDR-H2 comprising the amino acid sequence set forth in SEQ ID NO: 158; and CDR-H3 comprising the amino acid sequence set forth in SEQ ID NO: 160 and/or CDR-H1 comprising the amino acid sequence set forth in SEQ ID NO: 172; CDR-H2 comprising the amino acid sequence set forth in SEQ ID NO: 174; and CDR-H3 comprising the amino acid sequence set forth in SEQ ID NO: 176 and/or CDR-L1 comprising the amino acid sequence set forth in SEQ ID NO: 12; CDR-L2 comprising the amino acid sequence set forth in SEQ ID NO: 14; and CDR-L3 comprising the amino acid sequence set forth in SEQ ID NO: 16 and/or CDR-L1 comprising the amino acid sequence set forth in SEQ ID NO: 28; CDR-L2 comprising the amino acid sequence set forth in SEQ ID NO: 30; and CDR-L3 comprising the amino acid sequence set forth in SEQ ID NO: 32 and/or CDR-L1 comprising the amino acid sequence set forth in SEQ ID NO: 44; CDR-L2 comprising the amino acid sequence set forth in SEQ ID NO: 46; and CDR-L3 comprising the amino acid sequence set forth in SEQ ID NO: 48 and/or CDR-L1 comprising the amino acid sequence set forth in SEQ ID NO: 60; CDR-L2 comprising the amino acid sequence set forth in SEQ ID NO: 62; and CDR-L3 comprising the amino acid sequence set forth in SEQ ID NO: 64 and/or CDR-L1 comprising the amino acid sequence set forth in SEQ ID NO: 76; CDR-L2 comprising the amino acid sequence set forth in SEQ ID NO: 78; and CDR-L3 comprising the amino acid sequence set forth in SEQ ID NO: 80 and/or CDR-L1 comprising the amino acid sequence set forth in SEQ ID NO: 92; CDR-L2 comprising the amino acid sequence set forth in SEQ ID NO: 94; and CDR-L3 comprising the amino acid sequence set forth in SEQ ID NO: 96 and/or CDR-L1 comprising the amino acid sequence set forth in SEQ ID NO: 108; CDR-L2 comprising the amino acid sequence set forth in SEQ ID NO: 110; and CDR-L3 comprising the amino acid sequence set forth in SEQ ID NO: 112 and/or CDR-L1 comprising the amino acid sequence set forth in SEQ ID NO: 124; CDR-L2 comprising the amino acid sequence set forth in SEQ ID NO: 126; and CDR-L3 comprising the amino acid sequence set forth in SEQ ID NO: 128 and/or CDR-L1 comprising the amino acid sequence set forth in SEQ ID NO: 124; CDR-L2 comprising the amino acid sequence set forth in SEQ ID NO: 126; and CDR-L3 comprising the amino acid sequence set forth in SEQ ID NO: 128 and/or CDR-L1 comprising the amino acid sequence set forth in SEQ ID NO: 148; CDR-L2 comprising the amino acid sequence set forth in SEQ ID NO: 150; and CDR-L3 comprising the amino acid sequence set forth in SEQ ID NO: 152 and/or CDR-L1 comprising the amino acid sequence set forth in SEQ ID NO: 164; CDR-L2 comprising the amino acid sequence set forth in SEQ ID NO: 166; and CDR-L3 comprising the amino acid sequence set forth in SEQ ID NO: 168 and/or CDR-L1 comprising the amino acid sequence set forth in SEQ ID NO: 124; CDR-L2 comprising the amino acid sequence set forth in SEQ ID NO: 126; and CDR-L3 comprising the amino acid sequence set forth in SEQ ID NO: 128. In an embodiment of the invention, the antigen-binding protein comprises: (1) a heavy chain immunoglobulin variable region comprising a CDR-H1 comprising the amino acid sequence set forth in SEQ ID NO: 4; a CDR-H2 comprising the amino acid sequence set forth in SEQ ID NO: 6; and a CDR-H3 comprising the amino acid sequence set forth in SEQ ID NO: 8; and a light chain immunoglobulin variable region comprising a CDR-L1 comprising the amino acid sequence set forth in SEQ ID NO: 12; a CDR-L2 comprising the amino acid sequence set forth in SEQ ID NO: 14; and a CDR-L3 comprising the amino acid sequence set forth in SEQ ID NO: 16; (2) a heavy chain immunoglobulin variable region comprising a CDR-H1 comprising the amino acid sequence set forth in SEQ ID NO: 20; a CDR-H2 comprising the amino acid sequence set forth in SEQ ID NO: 22; and a CDR-H3 comprising the amino acid sequence set forth in SEQ ID NO: 24; and a light chain immunoglobulin variable region comprising a CDR-L1 comprising the amino acid sequence set forth in SEQ ID NO: 28; a CDR-L2 comprising the amino acid sequence set forth in SEQ ID NO: 30; and a CDR-L3 comprising the amino acid sequence set forth in SEQ ID NO: 32; (3) a heavy chain immunoglobulin variable region comprising a CDR-H1 comprising the amino acid sequence set forth in SEQ ID NO: 36; a CDR-H2 comprising the amino acid sequence set forth in SEQ ID NO: 38; and a CDR-H3 comprising the amino acid sequence set forth in SEQ ID NO: 40; and a light chain immunoglobulin variable region comprising a CDR-L1 comprising the amino acid sequence set forth in SEQ ID NO: 44; a CDR-L2 comprising the amino acid sequence set forth in SEQ ID NO: 46; and a CDR-L3 comprising the amino acid sequence set forth in SEQ ID NO: 48; (4) a heavy chain immunoglobulin variable region comprising a CDR-H1 comprising the amino acid sequence set forth in SEQ ID NO: 52; a CDR-H2 comprising the amino acid sequence set forth in SEQ ID NO: 54; and a CDR-H3 comprising the amino acid sequence set forth in SEQ ID NO: 56; and a light chain immunoglobulin variable region comprising a CDR-L1 comprising the amino acid sequence set forth in SEQ ID NO: 60; a CDR-L2 comprising the amino acid sequence set forth in SEQ ID NO: 62; and a CDR-L3 comprising the amino acid sequence set forth in SEQ ID NO: 64; (5) a heavy chain immunoglobulin variable region comprising a CDR-H1 comprising the amino acid sequence set forth in SEQ ID NO: 68; a CDR-H2 comprising the amino acid sequence set forth in SEQ ID NO: 70; and a CDR-H3 comprising the amino acid sequence set forth in SEQ ID NO: 72; and a light chain immunoglobulin variable region comprising a CDR-L1 comprising the amino acid sequence set forth in SEQ ID NO: 76; a CDR-L2 comprising the amino acid sequence set forth in SEQ ID NO: 78; and a CDR-L3 comprising the amino acid sequence set forth in SEQ ID NO: 80; (6) a heavy chain immunoglobulin variable region comprising a CDR-H1 comprising the amino acid sequence set forth in SEQ ID NO: 84; a CDR-H2 comprising the amino acid sequence set forth in SEQ ID NO: 86; and a CDR-H3 comprising the amino acid sequence set forth in SEQ ID NO: 88; and a light chain immunoglobulin variable region comprising a CDR-L1 comprising the amino acid sequence set forth in SEQ ID NO: 92; a CDR-L2 comprising the amino acid sequence set forth in SEQ ID NO: 94; and a CDR-L3 comprising the amino acid sequence set forth in SEQ ID NO: 96; (7) a heavy chain immunoglobulin variable region comprising a CDR-H1 comprising the amino acid sequence set forth in SEQ ID NO: 100; a CDR-H2 comprising the amino acid sequence set forth in SEQ ID NO: 102; and a CDR-H3 comprising the amino acid sequence set forth in SEQ ID NO: 104; and a light chain immunoglobulin variable region comprising a CDR-L1 comprising the amino acid sequence set forth in SEQ ID NO: 108; a CDR-L2 comprising the amino acid sequence set forth in SEQ ID NO: 110; and a CDR-L3 comprising the amino acid sequence set forth in SEQ ID NO: 112; (8) a heavy chain immunoglobulin variable region comprising a CDR-H1 comprising the amino acid sequence set forth in SEQ ID NO: 116; a CDR-H2 comprising the amino acid sequence set forth in SEQ ID NO: 118; and a CDR-H3 comprising the amino acid sequence set forth in SEQ ID NO: 120; and a light chain immunoglobulin variable region comprising a CDR-L1 comprising the amino acid sequence set forth in SEQ ID NO: 124; a CDR-L2 comprising the amino acid sequence set forth in SEQ ID NO: 126; and a CDR-L3 comprising the amino acid sequence set forth in SEQ ID NO: 128; (9) a heavy chain immunoglobulin variable region comprising a CDR-H1 comprising the amino acid sequence set forth in SEQ ID NO: 132; a CDR-H2 comprising the amino acid sequence set forth in SEQ ID NO: 134; and a CDR-H3 comprising the amino acid sequence set forth in SEQ ID NO: 136; and a light chain immunoglobulin variable region comprising a CDR-L1 comprising the amino acid sequence set forth in SEQ ID NO: 124; a CDR-L2 comprising the amino acid sequence set forth in SEQ ID NO: 126; and a CDR-L3 comprising the amino acid sequence set forth in SEQ ID NO: 128; (10) a heavy chain immunoglobulin variable region comprising a CDR-H1 comprising the amino acid sequence set forth in SEQ ID NO: 140; a CDR-H2 comprising the amino acid sequence set forth in SEQ ID NO: 142; and a CDR-H3 comprising the amino acid sequence set forth in SEQ ID NO: 144; and a light chain immunoglobulin variable region comprising a CDR-L1 comprising the amino acid sequence set forth in SEQ ID NO: 148; a CDR-L2 comprising the amino acid sequence set forth in SEQ ID NO: 150; and a CDR-L3 comprising the amino acid sequence set forth in SEQ ID NO: 152; (11) a heavy chain immunoglobulin variable region comprising a CDR-H1 comprising the amino acid sequence set forth in SEQ ID NO: 156; a CDR-H2 comprising the amino acid sequence set forth in SEQ ID NO: 158; and a CDR-H3 comprising the amino acid sequence set forth in SEQ ID NO: 160; and a light chain immunoglobulin variable region comprising a CDR-L1 comprising the amino acid sequence set forth in SEQ ID NO: 164; a CDR-L2 comprising the amino acid sequence set forth in SEQ ID NO: 166; and a CDR-L3 comprising the amino acid sequence set forth in SEQ ID NO: 168; or (12) a heavy chain immunoglobulin variable region comprising a CDR-H1 comprising the amino acid sequence set forth in SEQ ID NO: 172; a CDR-H2 comprising the amino acid sequence set forth in SEQ ID NO: 174; and a CDR-H3 comprising the amino acid sequence set forth in SEQ ID NO: 176; and a light chain immunoglobulin variable region comprising a CDR-L1 comprising the amino acid sequence set forth in SEQ ID NO: 124; a CDR-L2 comprising the amino acid sequence set forth in SEQ ID NO: 126; and a CDR-L3 comprising the amino acid sequence set forth in SEQ ID NO: 128. In an embodiment of the invention, the antigen-binding protein comprises (a) a heavy chain immunoglobulin that comprises the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and/or (b) a light chain immunoglobulin that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226. The present invention includes an antigen-binding protein (e.g., antibody or antigen-binding fragment thereof) comprising: (a) a heavy chain immunoglobulin variable region that comprises the amino acid sequence set forth in SEQ ID NO: 2, and a light chain immunoglobulin variable region that comprises the amino acid sequence set forth in SEQ ID NO: 10; (b) a heavy chain immunoglobulin variable region that comprises the amino acid sequence set forth in SEQ ID NO: 18, and a light chain immunoglobulin variable region that comprises the amino acid sequence set forth in SEQ ID NO: 26; (c) a heavy chain immunoglobulin variable region that comprises the amino acid sequence set forth in SEQ ID NO: 34, and a light chain immunoglobulin variable region that comprises the amino acid sequence set forth in SEQ ID NO: 42; (d) a heavy chain immunoglobulin variable region that comprises the amino acid sequence set forth in SEQ ID NO: 50, and a light chain immunoglobulin variable region that comprises the amino acid sequence set forth in SEQ ID NO: 58; (e) a heavy chain immunoglobulin variable region that comprises the amino acid sequence set forth in SEQ ID NO: 66, and a light chain immunoglobulin variable region that comprises the amino acid sequence set forth in SEQ ID NO: 74; (f) a heavy chain immunoglobulin variable region that comprises the amino acid sequence set forth in SEQ ID NO: 82, and a light chain immunoglobulin variable region that comprises the amino acid sequence set forth in SEQ ID NO: 90; (g) a heavy chain immunoglobulin variable region that comprises the amino acid sequence set forth in SEQ ID NO: 98, and a light chain immunoglobulin variable region that comprises the amino acid sequence set forth in SEQ ID NO: 106; (h) a heavy chain immunoglobulin variable region that comprises the amino acid sequence set forth in SEQ ID NO: 114, and a light chain immunoglobulin variable region that comprises the amino acid sequence set forth in SEQ ID NO: 122; (i) a heavy chain immunoglobulin variable region that comprises the amino acid sequence set forth in SEQ ID NO: 130, and a light chain immunoglobulin variable region that comprises the amino acid sequence set forth in SEQ ID NO: 122; (j) a heavy chain immunoglobulin variable region that comprises the amino acid sequence set forth in SEQ ID NO: 138, and a light chain immunoglobulin variable region that comprises the amino acid sequence set forth in SEQ ID NO: 146; (k) a heavy chain immunoglobulin variable region that comprises the amino acid sequence set forth in SEQ ID NO: 154, and a light chain immunoglobulin variable region that comprises the amino acid sequence set forth in SEQ ID NO: 162; and/or (1) a heavy chain immunoglobulin variable region that comprises the amino acid sequence set forth in SEQ ID NO: 170, and a light chain immunoglobulin variable region that comprises the amino acid sequence set forth in SEQ ID NO: 122—for example wherein the heavy chain immunoglobulin variable region is linked to an heavy chain constant region (e.g., IgG (e.g., IgG1 or IgG4)) and the light chain immunoglobulin variable region is linked to a light chain constant region (e.g., lambda or kappa). For example, the light and heavy chain constant regions are human constant regions. In an embodiment of the invention, the antigen-binding protein (e.g., antibody or antigen-binding fragment thereof) of the present invention comprises: (a) a heavy chain immunoglobulin that comprises the amino acid sequence set forth in SEQ ID NO: 180, and a light chain immunoglobulin that comprises the amino acid sequence set forth in SEQ ID NO: 182; (b) a heavy chain immunoglobulin that comprises the amino acid sequence set forth in SEQ ID NO: 184, and a light chain immunoglobulin that comprises the amino acid sequence set forth in SEQ ID NO: 186; (c) a heavy chain immunoglobulin that comprises the amino acid sequence set forth in SEQ ID NO: 188, and a light chain immunoglobulin that comprises the amino acid sequence set forth in SEQ ID NO: 190; (d) a heavy chain immunoglobulin that comprises the amino acid sequence set forth in SEQ ID NO: 192, and a light chain immunoglobulin that comprises the amino acid sequence set forth in SEQ ID NO: 194; (e) a heavy chain immunoglobulin that comprises the amino acid sequence set forth in SEQ ID NO: 196, and a light chain immunoglobulin that comprises the amino acid sequence set forth in SEQ ID NO: 198; (f) a heavy chain immunoglobulin that comprises the amino acid sequence set forth in SEQ ID NO: 200, and a light chain immunoglobulin that comprises the amino acid sequence set forth in SEQ ID NO: 202; (g) a heavy chain immunoglobulin that comprises the amino acid sequence set forth in SEQ ID NO: 204, and a light chain immunoglobulin that comprises the amino acid sequence set forth in SEQ ID NO: 206; (h) a heavy chain immunoglobulin that comprises the amino acid sequence set forth in SEQ ID NO: 208, and a light chain immunoglobulin that comprises the amino acid sequence set forth in SEQ ID NO: 210; (i) a heavy chain immunoglobulin that comprises the amino acid sequence set forth in SEQ ID NO: 212, and a light chain immunoglobulin that comprises the amino acid sequence set forth in SEQ ID NO: 214; (j) a heavy chain immunoglobulin that comprises the amino acid sequence set forth in SEQ ID NO: 216, and a light chain immunoglobulin that comprises the amino acid sequence set forth in SEQ ID NO: 218; (k) a heavy chain immunoglobulin that comprises the amino acid sequence set forth in SEQ ID NO: 220, and a light chain immunoglobulin that comprises the amino acid sequence set forth in SEQ ID NO: 222; and/or (1) a heavy chain immunoglobulin that comprises the amino acid sequence set forth in SEQ ID NO: 224, and a light chain immunoglobulin that comprises the amino acid sequence set forth in SEQ ID NO: 226.

Antigen-binding proteins of the present invention, may, in an embodiment of the invention, be characterized by one or more of the following properties:
Binds to human IL36R with a K_{D} of about 2.18 nM to about 13.9 nM at 25° C. or with a K_{D} of about 4.25 nM to about 29.5 nM at 37° C.;
Binds to Macaca fascicularis IL36R with a K_{D} of about 7.87 nM to about 34.4 nM at 25° C. or with a K_{D} of about 14.4 nM to about 58.2 nM at 37° C.;
Binds to human IL36R fused to a mouse IgG2a with a K_{D} of about 173 pM to about 5.79 nM at 25° C. or with a K_{D} of about 205 pM to about 28.7 nM at 37° C.;
Binds to human IL36R fused to IL1RAcP extracellular domain expressed with mouse IgG2a Fc tag with a K_{D} of about 212 pM to about 14 nM at 25° C. or with a K_{D} of about 264 pM to about 40.9 nM at 37° C.;
Competes with H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2 for binding to IL36R;
Blocks activation of one or more NFκB elements, which is/are fused to a reporter gene, in a host cell, by IL-36R (e.g., human or Macaca fascicularis) in the presence of IL-1RAcP and IL36R ligand;
Prevents or ameliorates skin inflammation or reduces skin thickness or total pathology score or reduces pro-inflammatory cytokine levels in a subject suffering from skin inflammation;
Prevents or ameliorates colitis or colon inflammation or reduces fecal levels of LCN2 polypeptide in a subject with such colitis or inflammation;
Protects residues (a) 113-119, 113-122, 116-119 and/or 116-122; and/or (b) 264-271, 267-271, 268-271, 268-276, 268-277 and/or 271-276, of human IL36R (IL-1RL2) set forth herein in SEQ ID NO: 227 (or the corresponding residues in wild-type IL-1RL2), when bound, from digestion with pepsin and/or Protease XIII and/or deuteration in the presence of deuterium;
Binds to IL36R (IL-1RL2) (e.g., human IL36R) at residues 113-119, 113-122, 116-119, 116-122, 264-271, 267-271, 268-271, 268-276, 268-277 and/or 271-276 of human IL36R comprising the amino acid sequence set forth herein in SEQ ID NO: 227 (or the corresponding residues in wild-type IL-1RL2);
Binds Domain II of IL36R (IL-1RL2) (e.g., human IL36R), e.g., with a coverage of about 80.0, 80.1, 81.0 or 81.5% or about 80-81 or 80-82% coverage; and/or
Binds a polypeptide comprising the amino acid sequence YKQILHLGKD (SEQ ID: 229) (amino acids 113-122 of SEQ ID NO: 227);
Inhibits IL36α, IL36β and/or IL36γ (e.g., at a concentration of about 10 nM), e.g., in in vitro epidermal keratinocytes, intestinal myofibroblasts and/or CD14+ monocytes, with an IC₅₀ of about 1, 2, 3, 4, 5 or 6 nM or 1-6 nM; and/or
Competitively inhibits IL36α, IL36β and/or IL367-mediated activation of NF□B (e.g., an NFκB response element (5×)-luciferase-IRES-GFP reporter in a cell such as HEK293) by IL36R; for example, as measured in a Schild Assay format.

Complexes comprising an IL36R polypeptide or antigenic fragment thereof complexed with an antigen-binding protein of the present invention (e.g., an antibody or antigen-binding fragment thereof, e.g., a human antibody or antigen-binding fragment thereof or a multispecific antibody) are also within the scope of the present invention.

Also provided by the present invention are methods for making an antigen-binding protein of the present invention (e.g., an antibody or antigen-binding fragment thereof, e.g., a human antibody or antigen-binding fragment thereof or a multispecific antibody) or an immunoglobulin chain thereof comprising: (a) introducing one or more polynucleotides encoding an immunoglobulin chain of said antigen-binding protein into a host cell (e.g., a Chinese hamster ovary (CHO) cell); (b) culturing the host cell under conditions favorable to expression of the polynucleotide; and (c) optionally, isolating the antigen-binding protein or immunoglobulin chain from the host cell and/or medium in which the host cell is grown. Antigen-binding proteins and immunoglobulin chains which are products of such a method are also part of the present invention.

The present invention also provides a polypeptide comprising: (a) CDR-H1, CDR-H2, and CDR-H3 of an immunoglobulin heavy chain variable region of an immunoglobulin chain that comprises the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and/or (b) CDR-L1, CDR-L2, and CDR-L3 of immunoglobulin light chain variable region of an immunoglobulin chain that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226; or, any amino acid sequence set forth herein, e.g., (c) the amino acid sequence set forth in a member selected from the group consisting of SEQ ID NO: 1-226. A polynucleotide encoding one or more (e.g., 2, e.g., a heavy and a light chain immunoglobulin set forth herein) of such polypeptides are also part of the present invention. Vectors, e.g., plasmids, comprising such a polynucleotide are also part of present invention. A host cell (e.g., a CHO cell) comprising any antigen-binding protein or immunoglobulin chain or polypeptide or polynucleotide or vector set forth herein is part of the present invention, e.g., wherein the polynucleotide and/or vector is integrated into a chromosome of the host cell or is ectopic.

A composition or kit comprising one or more of the antigen-binding proteins set forth herein (e.g., an antibody or antigen-binding fragment thereof, e.g., a human antibody or antigen-binding fragment thereof or a multispecific antibody), optionally in association with a further therapeutic agent (e.g., an anti-inflammatory agent, an anti-TNFalpha antibody or antigen-binding fragment thereof, an IL17 inhibitor, an IL23p19 inhibitor, an IL12p40 inhibitor, guselkumab, ustekinumab, brodalumab, ixekizumab, secukinumab, one or more human TNF receptors or fragments thereof linked to an immunoglobulin, infliximab, adalimumab, etanercept, dupilumab, sarilumab, tocilizumab, golimumab, abatacept, tofacitinib, abatacept, a non-steroidal anti-inflammatory drug (NSAID), ibuprofen, naproxen, acetaminophen, aspirin, celecoxib, cyclophosphamide, methotrexate, a corticosteroid, cortisone and prednisone, form part of the present invention.

Pharmaceutical compositions comprising an antigen-binding protein set forth herein (e.g., an antibody or antigen-binding fragment thereof, e.g., a human antibody or antigen-binding fragment thereof or a multispecific antibody) and a pharmaceutically acceptable carrier and, optionally, a further therapeutic agent, are also part of the present invention.

The present invention also provides a vessel or injection device (e.g., a pre-filled syringe) comprising an antigen-binding protein (e.g., an antibody or antigen-binding fragment thereof, e.g., a human antibody or antigen-binding fragment thereof or a multispecific antibody) or composition set forth herein.

The present invention further provides a method for treating or preventing an IL36R mediated disorder (e.g., an inflammatory or autoimmune disease or inflammatory bowel disease) in a subject in need thereof (e.g., a human), comprising administering (e.g., parenterally), to the subject, a therapeutically effective amount of antigen-binding protein as set forth herein (e.g., an antibody or antigen-binding fragment thereof, e.g., a human antibody or antigen-binding fragment thereof or a multispecific antibody), optionally in association with a further therapeutic agent (e.g., an anti-inflammatory agent).

The present invention also provides a method for administering an antigen-binding protein as set forth herein (e.g., an antibody or antigen-binding fragment thereof, e.g., a human antibody or antigen-binding fragment thereof or a multispecific antibody) into the body of a subject (e.g., a human) comprising injecting (e.g., subcutaneously, intravenously or intramuscularly) the antigen-binding protein into the body of the subject, optionally in association with a further therapeutic agent (e.g., an anti-inflammatory agent).

The present invention encompasses any polypeptide comprising an amino acid sequence which is set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174,176, 180, 184, 188, 192, 196, 200, 204, 208, 212, 216, 220, 224, 182, 186, 190, 194, 198, 202, 206, 210, 214, 218, 222 and/or 226 or a variant thereof.

The present invention includes any polynucleotide comprising a nucleotide sequence which is set forth in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173,175, 179, 183, 187, 191, 195, 199, 203, 207, 211, 215, 219, 223, 181, 185, 189, 193, 197, 201, 205, 209, 213, 217, 221 and/or 225 or a variant thereof.

### BRIEF DESCRIPTION OF THE FIGURES

FIG. 1. Sequence comparison between germline V_{H} (IGHV3-9*01/D1-7*01/J4*02, SEQ ID NO: 241) and V_{H} (SEQ ID NO: 34) of H4H14706P2, and between germline V_{L} (IGKV3-11*01/IGKJ4*01, SEQ ID NO: 242) and V_{L} (SEQ ID NO: 42) of H4H14706P2.

FIG. 2. Sequence comparison between germline V_{H} (IGHV3-9*01/D1-26*01/J4*02, SEQ ID NO: 243) and V_{H} (SEQ ID NO: 50) of H4H14708P2, and between germline V_{L} (IGKV3-11*01/IGKJ4*01, SEQ ID NO: 242) and V_{L} (SEQ ID NO: 58) of H4H14708P2.

FIGS. 3A-3F. Increasing concentration of H4H14706P2 and H4H14708P2 generated rightward shift of IL-36α (3A and 3D), IL-36β (3B and 3E), or IL-36γ (3C and 3F) dose response curves revealing competitive nature of inhibition by H4H14706P2 and H4H14708P2 (RLU, relative light units)

FIG. 4. H4H141708P2 and APE6155 pharmacokinetic analysis (concentration of antibody in serum over time) in cynomolgus monkeys dosed subcutaneously with 0.5 mg/kg or 5.0 mg/kg of antibody.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

An anti-IL36R “antigen-binding protein” is a single polypeptide (e.g., an ScFv (single chain variable fragment)) or complex of more than one polypeptide (e.g., a tetrameric IgG antibody) that binds specifically to the IL36 receptor at the IL1RL2 subunit (IL-1Rrp2). IL-36R, in the context of binding of an antigen-binding protein thereto, refers to IL-1RL2. In an embodiment of the invention, the antigen-binding protein is an antibody or antigen-binding fragment whether monospecific or multispecific (e.g., bispecific) or monovalent or multivalent (e.g., bivalent). A monovalent antigen-binding protein has a single antigen-binding domain whereas a bivalent antigen-binding protein has two antigen-binding domains.

A polynucleotide includes DNA and RNA. The present invention includes any polynucleotide of the present invention which is operably linked to a promoter or other expression control sequence.

In general, a “promoter” or “promoter sequence” is a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level.

Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell, the sequence directs RNA polymerase mediated transcription of the coding sequence into RNA, preferably mRNA, which then may be RNA spliced (if it contains introns) and, optionally, translated into a protein encoded by the coding sequence.

### Interleukin-36 Receptor (IL36R)

IL-36R is a member of the IL-1 receptor family that contains six receptor proteins that form four signaling complexes: IL-1RI, IL-18R, IL-33R, and IL-36R, and two decoy receptors and two negative regulators. IL-36R is a heterodimer that consists of a receptor subunit named IL-1Rrp2 (also known as IL-1RL2, Interleukin 1 receptor-like 2 or Interleukin 1 receptor-related protein 2) and a co-receptor subunit Interleukin-1 receptor accessory protein, IL-1RAcP. The receptor can recognize three different agonists, IL-36α, IL-36β, and IL-36γ (also known as IL-1F6, IL-1F8, and IL-1F9), to induce the expression of inflammatory cytokines. There are also two receptor antagonists, IL-36Ra and IL-38, which bind to IL-36 receptor and decrease the expression of inflammatory cytokines. IL-36α, IL-36β, and IL-36γ signal through the IL-36R/IL-1RAcP receptor to activate NF-κB and MAPKs, such as p38 and JNK, and promote inflammatory responses.

In an embodiment of the invention, the Homo sapiens IL1RL2 sequence is available under Genbank accession number NP_003845.2. In an embodiment of the invention, the amino acid sequence of Homo sapiens IL1RL2 is set forth in SEQ ID NO: 177.

In an embodiment of the invention, Homo sapiens IL-1RAcP sequence is available under Genbank accession no. NP_002173.1. In an embodiment of the invention, the amino acid sequence of Homo sapiens IL-1RAcP is set forth in SEQ ID NO: 178.

### Anti-IL36 Antibodies and Antigen-Binding Fragments Thereof

The present invention provides antigen-binding proteins, such as antibodies (e.g., human antibodies) and antigen-binding fragments thereof, that specifically bind to IL36R protein or an antigenic fragment thereof. Antigen-binding proteins that bind to the same epitope on IL36R as, or compete for binding to IL36R with any of the antigen-binding proteins set forth herein are also part of the present invention.

The term “antibody”, as used herein, refers to immunoglobulin molecules comprising four polypeptide chains, two heavy chains (HCs) and two light chains (LCs) inter-connected by disulfide bonds (i.e., “full antibody molecules”), as well as multimers thereof—for example H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2. In an embodiment of the invention, each heavy chain (HC) comprises a heavy chain variable region (“HCVR” or “V_{H}”) (e.g., SEQ ID NO: 2, 18, 34, 50, 66, 82, 98, 114, 130, 138, 154 or 170 or a variant thereof) and a heavy chain constant region (including domains CH1, CH2 and CH3); and each light chain (LC) comprises a light chain variable region (“LCVR or “V_{L}”) (e.g., SEQ ID NO: 10, 26, 42, 58, 74, 90, 106, 122, 146 or 162 or a variant thereof) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) are identical to the human germline sequences, or are naturally or artificially modified.

Typically, the variable domains of both the heavy and light immunoglobulin chains comprise three hypervariable regions, also called complementarity determining regions (CDRs), located within relatively conserved framework regions (FR). In general, from N-terminal to C-terminal, both light and heavy chains variable domains comprise FR1, CDR1, FR2, CDR2, FR3, CDR3 and FR4. In an embodiment of the invention, the assignment of amino acids to each domain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

For example, the present invention provides an antigen-binding protein that includes (a) a heavy chain immunoglobulin comprising the CDR-H1, CDR-H2 and CDR-H3 of a heavy chain immunoglobulin comprising an amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224 and at least 70, 80 or 90% amino acid sequence identity to the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; and (b) an light chain immunoglobulin comprising the CDR-L1, CDR-L2 and CDR-L3 of a light chain immunoglobulin comprising an amino acid sequence set forth in SEQ ID NO: 10, 26, 42, 58, 74, 90, 106, 122, 146, 162, 182, 186, 190, 194, 198, 202, 206, 210, 214, 218, 222 or 226 and at least 70, 80 or 90% amino acid sequence identity to the amino acid sequence set forth in SEQ ID NO: 10, 26, 42, 58, 74, 90, 106, 122, 146, 162, 182, 186, 190, 194, 198, 202, 206, 210, 214, 218, 222 or 226. In an embodiment of the invention, the antigen-binding protein includes (i) a heavy chain immunoglobulin that comprises CDR-H1, CDR-H2 and CDR-H3 of a heavy chain immunoglobulin that comprises the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224; or a variant thereof; and (ii) a light chain immunoglobulin that comprises CDR-L1, CDR-L2 and CDR-L3 of a light chain immunoglobulin that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226; or a variant thereof.

In an embodiment of the invention, an antigen-binding protein of the present invention includes a heavy chain immunoglobulin that comprises a V_{H} including CDR-H1, CDR-H2 and CDR-H3, wherein the:
CDR-H1 comprises the amino acid sequence set forth in SEQ ID NO: 4, 20, 36, 52, 68, 84, 100, 116, 132, 140, 156 or 172, or a variant thereof;
CDR-H2 comprises the amino acid sequence set forth in SEQ ID NO: 6, 22, 38, 54, 70, 86, 102, 118, 134, 142, 158 or 174, or a variant thereof; and
CDR-H3 comprises the amino acid sequence set forth in SEQ ID NO: 8, 24, 40, 56, 72, 88, 104, 120, 136, 144, 160 or 176, or a variant thereof; and
a light chain immunoglobulin that comprises a V_{L} including CDR-L1, CDR-L2 and CDR-L3, wherein the:
CDR-L1 comprises the amino acid sequence set forth in SEQ ID NO: 12, 28, 44, 60, 76, 92, 108, 124, 124, 148 or 164, or a variant thereof;
CDR-L2 comprises the amino acid sequence set forth in SEQ ID NO: 14, 30, 46, 62, 78, 94, 110, 126, 126, 150 or 166, or a variant thereof; and
CDR-L3 comprises the amino acid sequence set forth in SEQ ID NO: 16, 32, 48, 64, 80, 96, 112, 128, 128, 152 or 168, or a variant thereof.

The present invention includes monoclonal anti-IL36R antigen-binding proteins, e.g., antibodies and antigen-binding fragments thereof, as well as monoclonal compositions comprising a plurality of isolated monoclonal antigen-binding proteins. The term “monoclonal antibody” or “mAb”, as used herein, refers to a member of a population of substantially homogeneous antibodies, i.e., the antibody molecules comprising the population are identical in amino acid sequence except for possible naturally occurring mutations that may be present in minor amounts.

A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment of the invention, an anti-IL36R antigen-binding protein, e.g., antibody or antigen-binding fragment comprises a heavy chain constant domain, e.g., of the type IgA (e.g., IgA1 or IgA2), IgD, IgE, IgG (e.g., IgG1, IgG2, IgG3 and IgG4) or IgM. In an embodiment of the invention, an antigen-binding protein, e.g., antibody or antigen-binding fragment, comprises a light chain constant domain, e.g., of the type kappa or lambda.

The present invention includes human antigen-binding proteins. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See e.g., U.S. Pat. Nos. 8,502,018, 6,596,541 or 5,789,215. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject.

The present invention includes anti-IL36R chimeric antigen-binding proteins, e.g., antibodies and antigen-binding fragments thereof, and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81: 6851-6855).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library.

Recombinant anti-IL36R antigen-binding proteins, e.g., antibodies and antigen-binding fragments, disclosed herein may also be produced in an E. coli/T7 expression system. In this embodiment, nucleic acids encoding the anti-IL36R antibody immunoglobulin molecules of the invention (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the invention, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5;189(1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455. Thus, the present invention includes recombinant methods for making an anti-IL36R antigen-binding protein, such as an antibody or antigen-binding fragment thereof of the present invention, or an immunoglobulin chain thereof, comprising (i) introducing one or more polynucleotides (e.g., including the nucleotide sequence in any one or more of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183, 185, 187, 189, 191, 193, 195, 197, 199, 201, 203, 205, 207, 209, 211, 213, 215, 217, 219, 221, 223 and/or 225; or a variant thereof) encoding light and/or heavy immunoglobulin chains of the antigen-binding protein, e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2, for example, wherein the polynucleotide is in a vector; and/or integrated into a host cell chromosome and/or is operably linked to a promoter; (ii) culturing the host cell (e.g., CHO or Pichia or Pichia pastoris) under conditions favorable to expression of the polynucleotide and, (iii) optionally, isolating the antigen-binding protein (e.g., antibody or fragment) or chain from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods of the present invention include those wherein only a heavy immunoglobulin chain or only a light immunoglobulin chain or both (e.g., any of those discussed herein including mature fragments and/or variable domains thereof) are expressed in a cell. Such single chains are useful, for example, as intermediates in the expression of an antibody or antigen-binding fragment that includes such a chain. For example, the present invention also includes anti-IL36R antigen-binding proteins, such as antibodies and antigen-binding fragments thereof, comprising a heavy chain immunoglobulin (or variable domain thereof or comprising the CDRs thereof) encoded by a polynucleotide comprising the nucleotide sequences set forth in SEQ ID NO: 1, 17, 33, 49, 65, 81, 97, 113, 129, 137, 153, 169, 179, 183, 187, 191, 195, 199, 203, 207, 211, 215, 219 or 223; and a light chain immunoglobulin (or variable domain thereof or comprising the CDRs thereof) encoded by the nucleotide sequence set forth in SEQ ID NO: 9, 25, 41, 57, 73, 89, 105, 121, 145, 161, 181, 185, 189, 193, 197, 201, 205, 209, 213, 217, 221 or 225 which are the product of such production methods, and, optionally, the purification methods set forth herein. For example, in an embodiment of the invention, the product of the method is an anti-IL36R antigen-binding protein which is an antibody or fragment comprising a heavy chain immunoglobulin or V_{H} comprising the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224 and a light chain immunoglobulin or V_{L} comprising the amino acid sequence set forth in SEQ ID NO: 10, 26, 42, 58, 74, 90, 106, 122, 146, 162, 182, 186, 190, 194, 198, 202, 206, 210, 214, 218, 222 or 226.

In an embodiment of the invention, a method for making an anti-IL36R antigen-binding protein, e.g., antibody or antigen-binding fragment thereof, includes a method of purifying the antigen-binding protein, e.g., by column chromatography, precipitation and/or filtration. The product of such a method also forms part of the present invention.

Eukaryotic and prokaryotic host cells, including mammalian cells, may be used as hosts for expression of an anti-IL36R antigen-binding protein (e.g., antibody or antigen-binding fragment thereof). Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present invention includes an isolated host cell (e.g., a CHO cell or any type of host cell set forth above) comprising an antigen-binding protein, such as H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2; and/or a polynucleotide encoding one or more immunoglobulin chains thereof.

The present invention also includes a cell which is expressing IL36R or an antigenic fragment or fusion thereof (e.g., His₆, Fc and/or myc) which is bound by an antigen-binding protein of the present invention e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2, e.g., wherein the cell is in the body of a subject or is in vitro.

In addition, the present invention also provides a complex comprising an anti-IL36R antigen-binding protein, e.g., antibody or antigen-binding fragment, discussed herein complexed with IL36R polypeptide or an antigenic fragment thereof or fusion thereof and/or with a secondary antibody or antigen-binding fragment thereof (e.g., detectably labeled secondary antibody) that binds specifically to the anti-IL36R antibody or fragment. In an embodiment of the invention, the complex is in vitro (e.g., is immobilized to a solid substrate) or is in the body of a subject.

The term “specifically binds” refers to those antigen-binding proteins (e.g., mAbs) having a binding affinity to an antigen, such as IL36R protein, expressed as K_{D}, of at least about 58 nM (e.g., 10⁻⁹ M; 10⁻¹⁰ M, 10⁻¹¹ M, or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present invention includes antigen-binding proteins that specifically bind to IL36R protein. In an embodiment of the invention, an anti-IL36R antigen-binding protein comprises a K_{D} value, for binding to human and/or Macaca fascicularis IL36R, which value is set forth in any of Tables 4-1 to 4-8.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein. In an embodiment of the invention, the antigen-binding fragment comprises three or more CDRs of H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2 (e.g., CDR-H1, CDR-H2 and CDR-H3; or CDR-L1, CDR-L2 and CDR-L3).

An antigen-binding fragment of an antibody will, in an embodiment of the invention, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (X) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Antigen-binding proteins (e.g., antibodies and antigen-binding fragments) may be monospecific or multi-specific (e.g., bispecific). Multispecific antigen-binding proteins are discussed further herein.

In specific embodiments, antigen-binding proteins of the present invention (e.g., an antibody or antibody fragment) may be conjugated to a moiety such a ligand, a detectable label or a therapeutic moiety (“immunoconjugate”), a second anti-IL36R antibody, or any other therapeutic moiety.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

The present invention includes antigen-binding proteins, e.g., antibodies or antigen-binding fragments, that bind to the same epitope as an antigen-binding protein of the present invention (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2).

The term “epitope” refers to an antigenic determinant (e.g., on IL1RL2) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present invention includes antigen-binding proteins that compete for binding to IL36R, e.g., a variant IL36R epitope as discussed herein, with an antigen-binding protein of the present invention, e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2. The term “competes” as used herein, refers to an antigen-binding protein (e.g., antibody or antigen-binding fragment thereof) that binds to an antigen (e.g., IL1RL2) and inhibits or blocks the binding of another antigen-binding protein (e.g., antibody or antigen-binding fragment thereof) to the antigen. The term also includes competition between two antigen-binding proteins e.g., antibodies, in both orientations, i.e., a first antibody that binds and blocks binding of second antibody and vice versa. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between anti-IL36R antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the invention which is modified in some way retains the ability to specifically bind to IL36R, e.g., retains at least 10% of its IL36R binding activity (when compared to the parental antibody) when that activity is expressed on a molar basis. Preferably, an antibody or antigen-binding fragment of the invention retains at least 20%, 50%, 70%, 80%, 90%, 95% or 100% or more of the IL36R binding affinity as the parental antibody. It is also intended that an antibody or antigen-binding fragment of the invention may include conservative or non-conservative amino acid substitutions (referred to as “conservative variants” or “function conserved variants” of the antibody) that do not substantially alter its biologic activity.

A “variant” of a polypeptide, such as an immunoglobulin chain (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2 V_{H}, V_{L}, HC or LC), refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 180, 182, 184, 186, 188, 190, 192, 194, 196, 198, 200, 202, 204, 206, 208, 210, 212, 214, 216, 218, 220, 222, 224 or 226); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (e.g., any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173,175, 179, 183, 187, 191, 195, 199, 203, 207, 211, 215, 219, 223, 181, 185, 189, 193, 197, 201, 205, 209, 213, 217, 221 and/or 225); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

Anti-IL36R antigen-binding proteins, e.g., antibodies and antigen-binding fragments thereof of the present invention, in an embodiment of the invention, include a heavy chain immunoglobulin or variable region thereof having at least 70% (e.g., 80%, 85%, 90%, 95%, 99%) amino acid sequence identity to the amino acids set forth in SEQ ID NO: 2, 18, 34, 50, 66, 82, 98, 114, 130, 138, 154,170, 180, 184, 188, 192, 196, 200, 204, 208, 212, 216, 220 or 224; and/or a light chain immunoglobulin or variable region thereof having at least 70% (e.g., 80%, 85%, 90%, 95%, 99%) amino acid sequence identity to the amino acids set forth in SEQ ID NO: 10, 26, 42, 58, 74, 90, 106, 122, 146, 162, 182, 186, 190, 194, 198, 202, 206, 210, 214, 218, 222 or 226.

In addition, an anti-IL36R antigen-binding protein may include a polypeptide comprising an amino acid sequence that is set forth herein except for one or more (e.g., 1, 2, 3, 4, 5, 6, 7, 8, 9 or 10) mutations such as, for example, missense mutations (e.g., conservative substitutions), non-sense mutations, deletions, or insertions. For example, the present invention includes anti-IL36R antigen-binding proteins which include an immunoglobulin light chain (or V_{L}) variant comprising the amino acid sequence set forth in SEQ ID NO: 10, 26, 42, 58, 74, 90, 106, 122, 146, 162, 182, 186, 190, 194, 198, 202, 206, 210, 214, 218, 222 or 226 but having one or more of such mutations and/or an immunoglobulin heavy chain (or V_{H}) variant comprising the amino acid sequence set forth in SEQ ID NO: 2, 18, 34, 50, 66, 82, 98, 114, 130, 138, 154,170, 180, 184, 188, 192, 196, 200, 204, 208, 212, 216, 220 or 224 but having one or more of such mutations. In an embodiment of the invention, an anti-IL36R antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

Embodiments of the present invention also include antigen-binding proteins, e.g., anti-IL36R antibodies and antigen-binding fragments thereof, that comprise immunoglobulin V_{H}S and V_{L}S; or HCs and LCs, which comprise a variant amino acid sequence having 70% or more (e.g., 80%, 85%, 90%, 95%, 97% or 99%) overall amino acid sequence identity or similarity to the amino acid sequences of the corresponding V_{H}S, V_{L}s, HCs or LCs specifically set forth herein, but wherein the CDR-L1, CDR-L2, CDR-L3, CDR-H1, CDR-H2 and CDR-H3 of such immunoglobulins are not variants and comprise the amino acid sequences specifically set forth herein. Thus, in such embodiments, the CDRs within variant antigen-binding proteins are not, themselves, variants.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g. charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present invention includes anti-IL36R antigen-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45.

Anti-IL36R antigen-binding proteins set forth herein, e.g., comprising variant immunoglobulin chains, may exhibit one or more of the following properties:
Binds to human IL36R (e.g., IL1RL2) (e.g., fused to a myc-myc-His₆ tag) with a K_{D} of about 2.18 nM to about 13.9 nM, e.g., at 25° C. or with a K_{D} of about 4.25 nM to about 29.5 nM, e.g., at 37° C.;
Binds to Macaca fascicularis IL36R (e.g., IL1RL2) (e.g., fused to a myc-myc-His₆ tag) with a K_{D} of about 7.87 nM to about 34.4 nM, e.g., at 25° C. or with a K_{D} of about 14.4 nM to about 58.2 nM, e.g., at 37° C.;
Binds to human IL36R (e.g., IL1RL2) (e.g., fused to a mouse IgG2a) with a K_{D} of about 173 pM to about 5.79 nM, e.g., at 25° C. or with a K_{D} of about 205 pM to about 28.7 nM, e.g., at 37° C.;
Binds to human IL36R (e.g., IL1RL2) (e.g., fused to IL1RAcP extracellular domain expressed with mouse IgG2a Fe tag) with a K_{D} of about 212 pM to about 14 nM, e.g., at 25° C. or with a K_{D} of about 264 pM to about 40.9 nM, e.g., at 37° C.;
Competes with one or more of the following anti-IL36R antibodies: H4H14699P2; H4H14700P2; H4H14706P2; H4H14708P2; H4H14709P; H4H14728P; H4H14731P; H4H14732P2; H4H14734P2; H4H14757P; H4H14758P and/or H4H14760P2 for binding to IL36R (e.g., IL1RL2), optionally with the proviso that such anti-IL36R antibody or fragment which competes with H4H14699P2; H4H14700P2; H4H14706P2; H4H14708P2; H4H14709P; H4H14728P; H4H14731P; H4H14732P2; H4H14734P2; H4H14757P; H4H14758P and/or H4H14760P2 is not antibody APE3798; APE4086; APE5125/APE5100; APE5216; APE5281; APE5214/APE4881; APE5280; APE5257; APE5258/APE5076; APE5212; APE5213/5066; APE5211; APE5217/APE5060; APE3849; APE3850; APE5600; APE5598; APE5627; APE6064; APE6060; APE6157; APE6155/APE6917; APE6194; APE3847; APE5713; APE6083; APE6903/APE7247; APE6904; and/or APE6907 (e.g., APE6155) or an antigen-binding fragment thereof or antigen-binding protein comprising the CDRs or variable regions thereof (see WO2016/168542);
Blocks activation of NFκB by IL-36R (e.g., human or Macaca fascicularis) in the presence of IL-1RAcP and ligand such as hIL-36α, hIL-36β, and/or hIL-367, e.g., wherein the NFκB is in a host cell, such as HEK293, e.g., containing NFκB response element (5×)-luciferase-IRES-GFP, e.g., with an IC₅₀ of about 1×10⁻¹⁰ M-7×10⁻⁹ M;
Prevents or ameliorates skin inflammation (e.g., chronic or acute) or reduces skin thickness or total pathology score or reduces pro-inflammatory cytokine levels (e.g., KC-GRO, IL6, IL1b and/or TNFalpha) in a subject suffering from skin inflammation (e.g., chronic or acute), e.g., chemically-induced skin inflammation (e.g., imiquimod-induced), e.g., in a mouse such as a mouse displaying symptoms of human DITRA (Deficiency of Interleukin Thirty-six Receptor Antagonist) disease, which disease is described in e.g., Marrakchi et al., Interleukin-36-receptor antagonist deficiency and generalized pustular psoriasis, N Engl J Med 365:620-628 (2011)—e.g., relative to a subject not treated with such an antigen-binding protein; and/or
Prevents or ameliorates colitis or colon inflammation, e.g., chemically-induced colitis or colon inflammation, e.g., induced by dextran sulfate sodium (DSS) or oxazolone, or reduces fecal levels of LCN2 polypeptide in a subject with such colitis or inflammation e.g., in a mouse such as a DITRA mouse—e.g., relative to a subject not treated with such an antigen-binding protein.
Protects residues (a) 113-119, 113-122, 116-119 and/or 116-122; and/or (b) 264-271, 267-271, 268-271, 268-276, 268-277 and/or 271-276, of an IL36R polypeptide comprising the amino acid sequence set forth in SEQ ID NO: 227, when bound, from digestion with pepsin and/or Protease XIII (e.g., from Aspergillus saitoi) and/or deuteration in the presence of deuterium (e.g., D₂O);*
Binds to IL36R, for example, the IL1RL2 subunit thereof, at residues 113-119, 113-122, 116-119, 116-122, 264-271, 267-271, 268-271, 268-276, 268-277 and/or 271-276 of an IL36R polypeptide comprising the amino acid sequence set forth in SEQ ID NO: 227;*
Binds Domain II of IL36R, e.g., with a coverage of about 80.0, 80.1, 81.0 or 81.5%;
Binds a polypeptide comprising the amino acid sequence YKQILHLGKD (SEQ ID NO: 229) (amino acids 113-122 of SEQ ID NO: 227) and/or GVETHVSFREHNYL (SEQ ID NO: 230) (amino acids 264-277 of SEQ ID NO: 227)
Inhibits IL36α, IL36β and/or IL36γ (e.g., at a concentration of about 10 nM), e.g., in in vitro epidermal keratinocytes, intestinal myofibroblasts and/or CD14+ monocytes, with an IC₅₀ of about 1, 2, 3, 4, 5 or 6 nM or 1-6 nM; and/or
Competitively inhibits IL36α, IL36β and/or IL367-mediated activation of NFκB (e.g., an NFκB response element (5×)-luciferase-IRES-GFP reporter in a cell such as HEK293) by IL36R; for example, as measured in a Schild Assay format.
   *Includes antigen-binding proteins that bind to a native IL36R (IL1-RL2), e.g., as set forth under UniProt Accession No. Q9HB29, at residues corresponding to those set forth in the tagged IL36R polypeptide comprising the amino acid sequence set forth in SEQ ID NO: 227. See below:

113-119:
(SEQ ID NO: 228)
YKQILHL (amino acids 113-119 of SEQ ID NO: 227);
113-122:
(SEQ ID NO: 229)
YKQIHLGKD (amino acids 113-122 of SEQ ID NO: 227);
116-119:
(SEQ ID NO: 231)
ILHL (amino acids 116-119 of SEQ ID NO: 227);
116-122:
(SEQ ID NO: 232)
ILHLGKD (amino acids 116-122 of SEQ ID NO: 227);
264-271:
(SEQ ID NO: 233)
GVETHVSF (amino acids 264-271 of SEQ ID NO: 227);
267-271:
(SEQ ID NO: 234)
THVSF (amino acids 267-271 of SEQ ID NO: 227);
268-271:
(SEQ ID NO: 235)
HVSF (amino acids 268-271 of SEQ ID NO: 227);
268-276:
(SEQ ID NO: 236)
HVSFREHNL (amino acids 268-276 of SEQ ID NO: 227);
268-277:
(SEQ ID NO: 237)
HVSFREHNLY (amino acids 268-277 of SEQ ID NO: 227);
271-276:
(SEQ ID NO: 238)
FREHNL (amino acids 271-276 of SEQ ID NO: 227).

See residues highlighted below in human IL36R (IL1RL2):

(SEQ ID NO: 117)
MTGLVSLSYF PLSTRSCALQ SCRQPGLGMW
SLLLCGLSIA LPLSVTADGC KDIFMKNEIL
SASQPFAFNC TFPPITSGEV SVTWYKNSSK
IPVSKIIQSR IHQDETWILF LPMEWGDSGV
YQCVIKGRDS CHRIHVNLTV FEKHWCDTSI
GGLPNLSDEY KQILHLGKDD SLTCHLHFPK
SCVLGPIKWY KDCNEIKGER FTVLETRLLV
SNVSAEDRGN YACQAILTHS GKQYEVLNGI
TVSITERAGY GGSVPKIIYP KNHSIEVQLG
TTLIVDCNVT DTKDNTNLRC WRVNNTLVDD
YYDESKRIRE GVETHVSFRE HNLYTVNITF
LEVKMEDYGL PFMCHAGVST AYIILQLPAP
DFRAYLIGGL IALVAVAVSV VYIYNIFKID
IVLWYRSAFH STETIVDGKL YDAYVLYPKP
HKESQRHAVD ALVINILPEV LERQCGYKLF
IFGRDEFPGQ AVANVIDENV KLCRRLIVIV
VPESLGFGLL KNLSEEQIAV YSALIQDGMK
VILIELEKIE DYTVMPESIQ YIKQKHGAIR
WHGDFTEQSQ CMKTKFWKTV RYHMPPRRCR
PFPPVQLLQH TPCYRTAGPE LGSPRKKCTLTTG

The present invention includes “neutralizing” or “antagonist” anti-IL36R antigen-binding proteins, e.g., antibody or antigen-binding fragment, which includes molecules that inhibit an activity of IL36R to any detectable degree (e.g., IL36 ligand binding).

“H4H14699P2”, “H4H14700P2”, “H4H14706P2”, “H4H14708P2”, “H4H14709P”, “H4H14728P”, “H4H14731P”, “H4H14732P2”, “H4H14734P2”, “H4H14757P”, “H4H14758P” and “H4H14760P2” refer to antigen-binding proteins, e.g., antibodies and antigen-binding fragments thereof (including multispecific antigen-binding proteins), comprising the immunoglobulin heavy chain or variable region thereof (V_{H}) of SEQ ID NO: 2, 18, 34, 50, 66, 82, 98, 114, 130, 138, 154,170, 180, 184, 188, 192, 196, 200, 204, 208, 212, 216, 220 or 224 (or a variant thereof), respectively; and the immunoglobulin light chain or variable region thereof (V_{L}) of 10, 26, 42, 58, 74, 90, 106, 122, 146, 162, 182, 186, 190, 194, 198, 202, 206, 210, 214, 218, 222 or 226 (or a variant thereof), respectively; or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)), e.g., wherein the immunoglobulin chains, variable regions and/or CDRs comprise the specific amino acid sequences described below. In an embodiment of the invention, the V_{H} is linked to an IgG constant heavy chain domain (e.g., IgG1 or IgG4) and/or the V_{L} is linked to a lambda or kappa constant light chain domain.

Antibodies and antigen-binding fragments of the present invention comprise immunoglobulin chains including the amino acid sequences set forth herein as well as cellular and in vitro post-translational modifications to the antibody or fragment. For example, the present invention includes antibodies and antigen-binding fragments thereof that specifically bind to IL36R comprising heavy and/or light chain amino acid sequences set forth herein (e.g., CDR-H1, CDR-H2, CDR-H3, CDR-L1, CDR-L2 and/or CDR-L3) as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine is missing.

The present invention provides a vessel (e.g., a plastic or glass vial, e.g., with a cap or a chromatography column, hollow bore needle or a syringe cylinder) comprising an anti-IL36R antigen-binding protein of the present invention, e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2.

The present invention also provides an injection device comprising one or more antigen-binding proteins (e.g., antibody or antigen-binding fragment) that bind specifically to IL36R, e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2, or a pharmaceutical composition thereof. The injection device may be packaged into a kit. An injection device is a device that introduces a substance into the body of a subject via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the antibody or fragment or a pharmaceutical composition thereof), a needle for piecing skin and/or blood vessels for injection of the fluid; and a plunger for pushing the fluid out of the cylinder and through the needle bore.

The present invention further provides methods for administering an anti-IL36R antigen-binding protein of the present invention, e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2, to a subject, comprising introducing the antigen-binding protein into the body of the subject (e.g., a human), for example, parenterally. For example, the method comprises piercing the body of the subject with a needle of a syringe and injecting the antigen-binding protein into the body of the subject, e.g., into the vein, artery, tumor, muscular tissue or subcutis of the subject.

### Preparation of Human Antibodies

Methods for generating human antibodies in transgenic mice are known in the art. Any such known methods can be used in the context of the present invention to make human antibodies that specifically bind to IL36R (e.g., IL1RL2). In certain embodiments of the invention, the antibodies of the invention are obtained from mice immunized with IL36R (e.g., IL1RL2 polypeptide or an immunogenic fragment thereof), or with a live attenuated or inactivated virus, or with DNA encoding the protein or fragment thereof. Alternatively, IL36R may be produced using standard biochemical techniques and modified and used as immunogen. In certain embodiments of the invention, the immunogen may be an IL36R (e.g., IL1RL2) polypeptide vaccine. In certain embodiments, one or more booster injections may be administered. In certain embodiments, the immunogen may be a recombinant IL36R polypeptide (e.g., IL1RL2) expressed in E. coli or in any other eukaryotic or mammalian cells such as Chinese hamster ovary (CHO) cells.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to IL36R can be initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

### Anti-IL36R Antibodies Comprising Fc Variants

According to certain embodiments of the present invention, anti-IL36R antigen-binding proteins, e.g., antibodies or antigen-binding fragments, are provided comprising an Fc domain comprising one or more mutations, which, for example, enhance or diminish antibody binding to the FcRn receptor, e.g., at acidic pH as compared to neutral pH. For example, the present invention includes anti-IL36R antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-IL36R antigen-binding proteins, e.g., antibodies or antigen-binding fragments, comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 3111 (e.g., P257I and Q3111); 257I and 434H (e.g., P257I and N434H); 376V and 434H (e.g., D376V and N434H); 307A, 380A and 434A (e.g., T307A, E380A and N434A); and 433K and 434F (e.g., H433K and N434F).

Anti-IL36R antigen-binding proteins, e.g., antibodies and antigen-binding fragments thereof, that comprise a V_{H} and/or V_{L} as set forth herein comprising any possible combinations of the foregoing Fc domain mutations, are contemplated within the scope of the present invention.

The present invention also includes anti-IL36R antigen-binding proteins, antibodies or antigen-binding fragments, comprising a V_{H} set forth herein and a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., WO2014/022540).

### Multispecific Antigen-Binding Proteins

The present invention includes anti-IL36R antigen-binding proteins, e.g., antibodies and antigen-binding fragments thereof, as well as methods of use thereof and methods of making such antigen-binding proteins. The term “anti-IL36R” antigen-binding protein, e.g., antibodies or antigen-binding fragments, includes multispecific (e.g., bispecific or biparatopic) molecules that include at least one first antigen-binding domain that specifically binds to IL36R (e.g., IL1RL2) (e.g., an antigen-binding domain from H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2) and at least one second antigen-binding domain that binds to a different antigen or to an epitope in IL36R which is different from that of the first antigen-binding domain (e.g., IL23-p19, IL12/IL23-p40, TNFalpha, IL-22, MADCAM, a4b7, CCR9, and/or CCR6). In an embodiment of the invention, the first and second epitopes overlap. In another embodiment of the invention, the first and second epitopes do not overlap.

“H4H14699P2”; “H4H14700P2”; “H4H14706P2”; “H4H14708P2”; “H4H14709P”; “H4H14728P”; “H4H14731P”; “H4H14732P2”; “H4H14734P2”; “H4H14757P”; “H4H14758P” or “H4H14760P2” includes a multispecific molecules, e.g., antibodies or antigen-binding fragments, that include the HCDRs and LCDRs, V_{H} and V_{L}, or HC and LC of H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2, respectively and one or more antigen-binding domains that bind to a different epitope.

In an embodiment of the invention, an antigen-binding domain that binds specifically to IL36R (e.g., IL1RL2), which may be included in a multispecific molecule, comprises:

(1)
(i) a heavy chain variable domain (V_{H}) sequence that comprises CDR-H1, CDR-H2 and CDR-H3 from an immunoglobulin heavy chain selected from: H4H14699P2; H4H14700P2; H4H14706P2; H4H14708P2; H4H14709P; H4H14728P; H4H14731P; H4H14732P2; H4H14734P2; H4H14757P; H4H14758P and H4H14760P2, and
(ii) a light chain variable domain (V_{L}) sequence that comprises CDR-L1, CDR-L2 and CDR-L3 from an immunoglobulin heavy chain selected from: H4H14699P2; H4H14700P2; H4H14706P2; H4H14708P2; H4H14709P; H4H14728P; H4H14731P; H4H14732P2; H4H14734P2; H4H14757P; H4H14758P and H4H14760P2, respectively; or,
   (2)
(i) a heavy chain variable domain (V_{H}) sequence selected from: H4H14699P2; H4H14700P2; H4H14706P2; H4H14708P2; H4H14709P; H4H14728P; H4H14731P; H4H14732P2; H4H14734P2; H4H14757P; H4H14758P and H4H14760P2; and
(ii) a light chain variable domain (V_{L}) sequence selected from: H4H14699P2; H4H14700P2; H4H14706P2; H4H14708P2; H4H14709P; H4H14728P; H4H14731P; H4H14732P2; H4H14734P2; H4H14757P; H4H14758P and H4H14760P2, respectively; and
one or more antigen-binding domains that bind to a different epitope.

In an embodiment of the invention, the multispecific antibody or fragment includes more than two different binding specificities (e.g., a trispecific molecule), for example, one or more additional antigen-binding domains which are the same or different from the first and/or second antigen-binding domain.

In one embodiment of the invention, a bispecific antigen-binding fragment comprises a first scFv (e.g., comprising V_{H} and V_{L} of H4H14699P2; H4H14700P2; H4H14706P2; H4H14708P2; H4H14709P; H4H14728P; H4H14731P; H4H14732P2; H4H14734P2; H4H14757P; H4H14758P and H4H14760P2) having binding specificity for a first epitope (e.g., IL36R) and a second scFv having binding specificity for a second, different epitope. For example, in an embodiment of the invention, the first and second scFv are tethered with a linker, e.g., a peptide linker (e.g., a GS linker such as (GGGGS)ₙ (SEQ ID NO: 177) wherein n is, for example, 1, 2, 3, 4, 5, 6, 7, 8, 9 or 10).

Other bispecific antigen-binding fragments include an F(ab)₂ of a bispecific IgG antibody which comprises the heavy and light chain CDRs of H4H14699P2; H4H14700P2; H4H14706P2; H4H14708P2; H4H14709P; H4H14728P; H4H14731P; H4H14732P2; H4H14734P2; H4H14757P; H4H14758P and H4H14760P2 and of another antibody that binds to a different epitope.

### Immunoconjugates

The invention encompasses anti-IL36R antigen-binding proteins, e.g., antibodies or antigen-binding fragments, conjugated to another moiety, e.g., a therapeutic moiety (an “immunoconjugate”). In an embodiment of the invention, an anti-IL36R antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a radioactive agent, a reporter moiety, an enzyme, a peptide, a protein or a therapeutic agent.

### Therapeutic and Prophylactic Methods

The present invention provides methods for treating or preventing an IL-36R-mediated disease by administering a therapeutically effective amount of anti-IL36R antigen-binding protein, e.g., antibody or antigen-binding fragment, (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2) to a subject (e.g., a human) in need of such treatment or prevention.

“Treat” or “treating” means to administer an anti-IL36R antigen-binding protein, e.g., antibody or antigen-binding fragment of the present invention (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), to a subject, having an IL36R-mediated disease, such that one or more signs and/or symptoms and/or clinical indicia of the IL36R-mediated disease regresses or is eliminated and/or the progression thereof is inhibited (e.g., the disease in the subject is stabilized, reduced or eliminated).

“Preventing” an IL36R-mediated disease means to administer anti-IL36R antigen-binding protein, e.g., antibody or antigen-binding fragment of the present invention (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), to a subject, prior to manifestation of the disease in the body of the subject.

Interleukin IL-36RN is an IL-1 cytokine family member that antagonizes the proinflammatory signals of IL-36alpha, IL-36beta and IL-36gamma at the IL-36R.

An IL-36R-mediated disease is any disease which is caused or exacerbated by an activity of IL-36R (e.g., activation of downstream inflammatory signaling via NFκB and MAP kinases due to receptor binding of ligand such as IL36γ, IL36 γ and/or IL36 γ), for example, due to a deficiency in an IL36R antagonist (e.g., IL-36RN). In an embodiment of the invention, a mutation in IL36RN underlies the IL-36R-mediated disease. An example of such a disease is an autoimmune and/or inflammatory disorder. In embodiment of the invention, the IL-36R-mediated disease treated with an anti-IL36R antigen-binding protein is an inflammatory disorder, an autoimmune disorder, deficiency of interleukin IL-36 receptor antagonist (DITRA) syndrome, impetigo herpetiformis, acrodermatitis, a skin neutrophilic pustular disease, psoriasis, a pustular disease, generalized pustular psoriasis (GPP; e.g., familial or sporadic), psoriasis vulgaris/plaque psoriasis, palmoplantar pustular psoriasis (PPPP), palmoplantar pustulosis (PPP), colitis, inflammatory bowel disease, ulcerative colitis, Crohn's disease, chemically-induced colitis, inflammation, airway inflammation (e.g., neutrophilic airway inflammation, COPD (chronic obstructive pulmonary disease) or asthma), joint inflammation (e.g., ankylosing spondylitis, rheumatoid arthritis or psoriatic arthritis), kidney inflammation, alopecia areata, skin inflammation (e.g., chemically-induced skin inflammation, psoriasis, pustular psoriasis, generalized pustular psoriasis, palmoplantar pustulosis, palmo-plantar pustular psoriasis, psoriasis vulgaris or psoriatic skin lesions), acanthosis, hyperkeratosis, kindler syndrome, systemic lupus erythematosus (SLE), nephrotic syndrome, ANCA (anti-neutrophil cytoplasmic antibody)-associated vasculopathies, tubulointerstitial lesions and glomerulonephritis.

An inflammatory disorder is a disorder characterized by uncontrolled or unwanted inflammation which may cause destruction of healthy tissue.

An autoimmune disorder is a condition in which one's immune system mistakenly attacks one's own body.

Impetigo herpetiformis (IH) is among rare dermatosis of pregnancy, which is currently considered as a form of generalized pustular psoriasis. In an embodiment of the invention, a mutation in IL36RN underlies the IH.

Acrodermatitis is a skin condition that may affect children, e.g., between the ages of 3 months and 15 years, which is characterized by itchy red or purple blisters on the body, bloated abdomen, fever, and swollen, sore lymph nodes. The cause of acrodermatitis may be viral.

Mutations of IL-36 receptor antagonists (e.g., IL-36Ra) are present in a high proportion of patients with GPP and acrodermatitis continua. In an embodiment of the invention, a mutation in IL36RN underlies the acrodermatitis.

Psoriasis is an autoimmune disease that causes skin plaques, which are itchy or sore patches of thick, red, dry skin. The most common form of psoriasis is psoriasis vulgaris (plaque psoriasis) which is characterized by well-defined plaques of red raised skin that can appear on any area of skin, including the knees, elbows, scalp and trunk. A flaky silvery white buildup on top of the plaques is called scale; it is composed of dead skin cells. This scale comes loose and sheds constantly from the plaques. Skin symptoms include pain, itching and cracking.

Generalized pustular psoriasis (GPP) is a severe form of psoriasis. Individuals with GPP typically have repeated episodes in which large areas of skin become red and inflamed and develop small pus-filled blisters (pustules). A portion of subjects with GPP suffer from plaques. The skin problems can be accompanied by fever, extreme tiredness (fatigue), muscle weakness, an increased number of white blood cells, and other signs of inflammation throughout the body (systemic inflammation). IL-36 cytokine appears to play a role in the development of GPP. In an embodiment of the invention, a mutation in IL36RN underlies the GPP.

Palmoplantar pustular psoriasis (PPPP; 4P) is a form of localized pustular psoriasis characterized by plaques and pustules occurring on palmar and plantar surfaces of the skin. PPPP may be associated with homozygous or compound heterozygous IL36RN gene mutations leading to aberrations in IL-36R antagonist function. In an embodiment of the invention, a mutation in IL36RN underlies the PPPP.

Palmoplantar pustulosis (PPP; 3P) is an immune-mediated disorder that causes blister-like pustules to show up on the palms of your hands and the soles of your feet. Generally, subjects with PPP do not suffer from plaques. In an embodiment of the invention, a mutation in IL36RN underlies the PPP.

Deficiency of interleukin IL-36 receptor antagonist (DITRA) syndrome is a rare autosomal recessive disease caused by mutations in IL36RN. DITRA is a rare, genetic, auto-inflammatory syndrome with immune deficiency disease characterized by recurrent and severe flares of generalized pustular psoriasis associated with high fever, asthenia, and systemic inflammation, due to IL36R antagonist deficiency. Psoriatic nail changes (e.g., pitting and onychomadesis) and ichthyosis may occasionally be associated. See Marrakchi et al., New Engl J. Med. 365(7): 620-628 (2011). In an embodiment of the invention, a mutation in IL36RN underlies the DITRA.

An inflammatory disease is a condition characterized by abnormal inflammation at one or more sites within the body of a subject. An autoimmune disease is a condition characterized by the abnormal attack of the subject's body tissue by the subject's own immune system.

ANCA-associated vasculopathies (AAV) are inflammatory disorders that include Granulomatosis with polyangiitis (formerly Wegener's), microscopic polyangiitis, and EGPA/Churg Strauss. These conditions are characterized by chronic inflammation leading to blockages of blood vessels and diminished blood flow to vital organs like the kidney.

Inflammatory bowel disease (IBD) is a term that includes two conditions (Crohn's disease and ulcerative colitis) that are characterized by chronic inflammation of the gastrointestinal (GI) tract.

Neutrophilic airway inflammation is inflammation of the airway which is mediated by the influx of neutrophils into the lungs. Signs and symptoms of neutrophilic airway inflammation include asthma and wheezing.

Chronic obstructive pulmonary disease (COPD) is a chronic inflammatory lung disease that causes obstructed airflow from the lungs. Signs and symptoms include breathing difficulty, cough, mucus (sputum) production and wheezing.

Ankylosing spondylitis (AS) is a disease characterized by long term inflammation of the spine (e.g., the sacroiliac (SI) joints and the axial skeleton). Over time, AS can cause some of the vertebrae in your spine to fuse. Symptoms include pain and stiffness in your lower back and hips.

Rheumatoid arthritis is an autoimmune condition characterized by joint inflammation. Symptoms include tender, warm, swollen joints; joint stiffness, fatigue, fever and weight loss.

Psoriatic arthritis is a form of arthritis that affects some people who have psoriasis. Symptoms can include swollen fingers and toes, foot pain and lower back pain.

Alopecia areata is spot baldness characterized by small bald patches on the body.

Acanthosis is diffuse epidermal thickening (hyperplasia) of the stratum spinosum (prickle cell layer) of the skin which may appear to be darker than other skin. Hyperkeratosis is a thickening of the outer layer of the skin.

Hyperkeratosis is the thickening of skin often due to irritation from the sun, chemicals or frequent friction or pressure. The skin thickening typically occurs in the outer layer of the skin, which contains a tough, protective protein called keratin.

Kindler syndrome is an autosomal recessive genodermatosis characterized by congenital acral skin blistering, photosensitivity, progressive poikiloderma, and diffuse cutaneous atrophy.

Mucosal manifestations are common, with frequent involvement of the oral mucosa, gingiva, and gastrointestinal tract.

Systemic lupus erythematosus (SLE) is an autoimmune disease. In this disease, the body's immune system mistakenly attacks healthy tissue. SLE can affect the skin, joints, kidneys, brain, and other organs.

Nephrotic syndrome is a kidney disorder that causes your body to excrete too much protein in your urine. Nephrotic syndrome is typically caused by damage to the clusters of small blood vessels in the kidneys that filter waste and excess water from your blood. Nephrotic syndrome symptoms may include swelling (edema), particularly in the feet and ankles, foamy urine, weight gain (from fluid retention), fatigue and loss of appetite.

Glomerulonephritis is inflammation of kidney glomeruli. Symptoms include ink or cola-colored urine from red blood cells in your urine (hematuria), foamy urine (due to proteinuria), high blood pressure (hypertension), fluid retention (edema).

An effective or therapeutically effective dose of anti-IL36R antigen-binding protein, e.g., antibody or antigen-binding fragment (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), for treating or preventing an IL-36R-mediated disease refers to the amount of the antibody or fragment sufficient to alleviate one or more of the clinical indicia, signs and/or symptoms of the disease in the treated subject, whether by inducing the regression or elimination of such indicia, signs and/or symptoms or by inhibiting the progression of such indicia, signs and/or symptoms. The dose amount may vary depending upon the age and the size of a subject to be administered, target disease, conditions, route of administration, and the like. In an embodiment of the invention, an effective or therapeutically effective dose of antibody or antigen-binding fragment thereof of the present invention, for treating or preventing IL36R mediated disease, e.g., in an adult human subject, is about 1 mg/kg or more, e.g., about 1 mg/kg to about 25 mg/kg. Depending on the severity of the infection, the frequency and the duration of the treatment can be adjusted. In certain embodiments, the antigen-binding protein of the present invention can be administered at an initial dose, followed by one or more secondary doses. In certain embodiments, the initial dose may be followed by administration of a second or a plurality of subsequent doses of antigen-binding protein in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

As used herein, the term “subject” refers to a mammal (e.g., rat, mouse, cat, dog, cow, sheep, horse, goat, rabbit), preferably a human, for example, in need of prevention and/or treatment of an IL-36R-mediated disease. The subject may have an IL-36R-mediated disease or be predisposed to developing such a disease. In an embodiment of the invention, the subject has a homozygous or heterozygous IL36RN mutation genotype.

The present invention encompasses methods for administering an anti-IL36R antigen-binding protein to a subject at risk of developing an IL36R-mediated disease. For example, in an embodiment of the invention, the disease is a skin inflammatory disease or colon inflammatory disease. Example 5 herein demonstrated that skin inflammation diseases could be prevented in a DITRA-like mouse model prior to exposure to imiquimod and the development of skin inflammation symptoms. In an embodiment of the invention, an IL36R-mediated disease (e.g., skin inflammation) is prevented by administration of a prophylactic dose of antigen-binding protein to a subject prior to any clinically significant inflammation, e.g., skin inflammation or any increase in inflammation-induced skin thickness, in total pathology score (as discussed herein) or in the presence of pro-inflammatory cytokines, such as KC-GRO, IL-6, IL-1beta or TNFalpha, in the skin. In an embodiment of the invention, a dose of anti-IL36R antigen-binding protein of the invention for preventing an IL36R-mediated disease is from about 1 mg/kg to about 10 mg/kg.

### Combinations and Pharmaceutical Compositions

The present invention provides compositions that include anti-IL36R antigen-binding proteins and one or more ingredients; as well as methods of use thereof and methods of making such compositions.

To prepare pharmaceutical compositions of the anti-IL36R antigen-binding proteins, e.g., antibodies and antigen-binding fragments thereof (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the invention, the pharmaceutical composition is sterile. Such compositions are part of the present invention.

Pharmaceutical compositions of the present invention include pharmaceutically acceptable carriers, diluents, excipients and/or stabilizers, such as, for example, water, buffering agents, stabilizing agents, preservatives, isotonifiers, non-ionic detergents, antioxidants and/or other miscellaneous additives.

The scope of the present invention includes desiccated, e.g., freeze-dried, compositions comprising an anti-IL36R antigen-binding protein, e.g., antibody or antigen-binding fragment thereof (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), or a pharmaceutical composition thereof that includes a pharmaceutically acceptable carrier but substantially lacks water.

In a further embodiment of the invention, a further therapeutic agent that is administered to a subject in association with an anti-IL36R antigen-binding protein, e.g., antibody or antigen-binding fragment thereof (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), disclosed herein is administered to the subject in accordance with the Physicians' Desk Reference 2003 (Thomson Healthcare; 57^{th} edition (Nov. 1, 2002)).

The mode of administration of an antigen-binding protein or composition thereof can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial.

The present invention provides methods for administering an anti-IL36R antigen-binding protein, e.g., antibody or antigen-binding fragment thereof (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2) to a subject, comprising introducing the protein or a pharmaceutical composition or combination thereof into the body of the subject. For example, in an embodiment of the invention, the method comprises piercing the body of the subject, e.g., with a needle of a syringe, and injecting the antigen-binding protein or a pharmaceutical composition or combination thereof into the body of the subject, e.g., into the vein, artery, tumor, muscular tissue or subcutis of the subject.

The present invention provides a vessel (e.g., a plastic or glass vial, e.g., with a cap or a chromatography column, hollow bore needle or a syringe cylinder) comprising any of the anti-IL36R antigen-binding proteins, e.g., antibodies or antigen-binding fragments thereof (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), or a pharmaceutical composition comprising a pharmaceutically acceptable carrier or combination thereof.

The present invention includes combinations including an anti-IL36R antigen-binding protein, e.g., antibody or antigen-binding fragment thereof of the present invention (e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2), in association with one or more further therapeutic agents. The anti-IL36R antigen-binding protein and the further therapeutic agent can be in a single composition or in separate compositions. For example, in an embodiment of the invention, the further therapeutic agent is an anti-inflammatory drug. In an embodiment of the invention, the further therapeutic agent is another anti-IL35R antigen-binding protein, an IL17 inhibitor, an IL23p19 inhibitor, an IL12p40 inhibitor, guselkumab, ustekinumab, brodalumab, ixekizumab, secukinumab, an anti-TNFalpha antibody or antigen-binding fragment thereof, one or more human TNF receptors or fragments thereof linked to an immunoglobulin such as an Fc portion of a human IgG1, infliximab, adalimumab, etanercept, dupilumab, sarilumab, tocilizumab, golimumab, abatacept, tofacitinib, abatacept, a non-steroidal anti-inflammatory drug (NSAID), ibuprofen, naproxen, acetaminophen, aspirin, celecoxib, cyclophosphamide, methotrexate, a corticosteroid, cortisone or prednisone.

Methods for treating or preventing an IL-36-mediated disease in a subject in need of said treatment or prevention by administering an anti-IL36R antigen-binding protein, e.g., H4H14699P2; H4H14700P2; H4H14706P2; H4H14708P2; H4H14709P; H4H14728P; H4H14731P; H4H14732P2; H4H14734P2; H4H14757P; H4H14758P or H4H14760P2, in association with a further therapeutic agent are part of the present invention.

The term “in association with” indicates that components, an anti-IL36R antigen-binding protein, e.g., antibody or antigen-binding fragment thereof of the present invention, along with another agent such as methotrexate, can be formulated into a single composition, e.g., for simultaneous delivery, or formulated separately into two or more compositions (e.g., a kit including each component). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route.

### EXAMPLES

The following examples are put forth so as to provide those of ordinary skill in the art with a complete disclosure and description of how to make and use the methods and compositions of the invention, and are not intended to limit the scope of what the inventors regard as their invention.

### Example 1: Generation of Human Antibodies that Specifically Bind to IL-36R

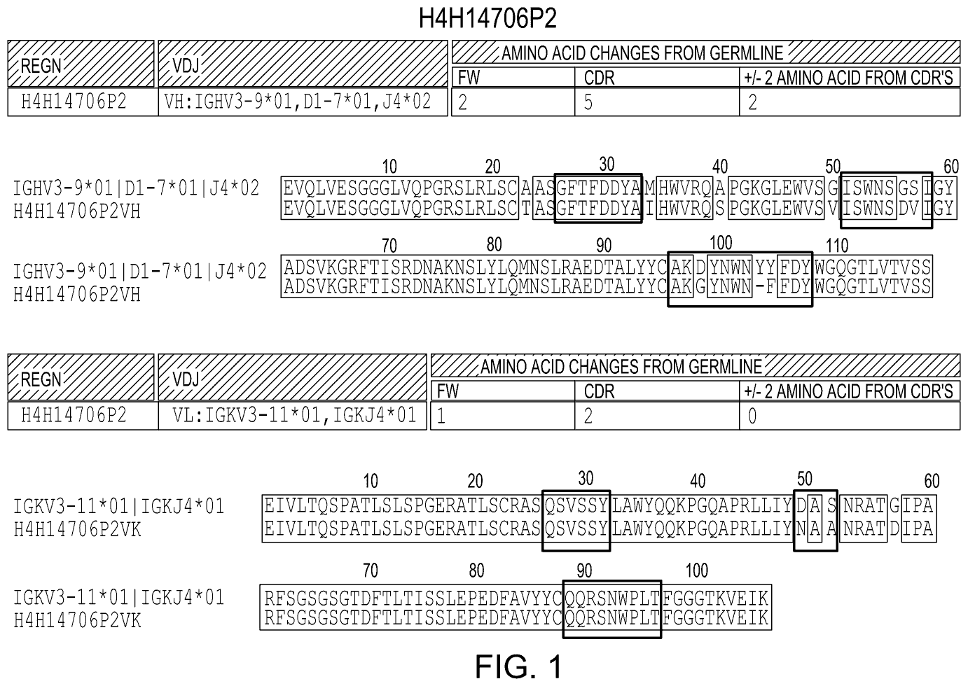

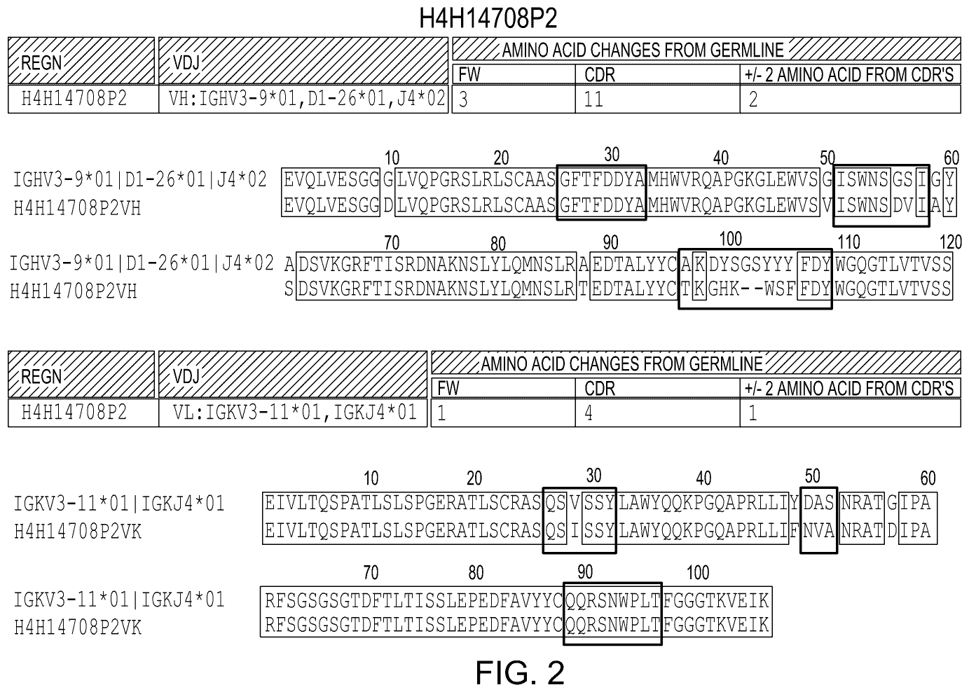

Anti-IL36R antibodies were obtained by immunizing a VELOCIMMUNE mouse (i.e., an engineered mouse comprising DNA encoding human immunoglobulin heavy and kappa light chain variable regions) with a DNA immunogen comprising the full length IL36R (IL-1RL2) sequence. The antibody immune response was monitored by an IL36R-specific immunoassay and fully human anti-IL36R antibodies were isolated and purified. Two exemplary comparisons between the V_{H} and V_{L} of antibodies generated as set forth herein and their respective germlines are set forth in FIG. 1 and FIG. 2.

TABLE 1
Immunoglobulin chain sequences of the present invention*

| Antibody | | VH | | CDR1 | | CDR2 | | CDR3 | |
|---|---|---|---|---|---|---|---|---|---|
| # | Name | DNA | PEP | DNA | PEP | DNA | PEP | DNA | PEP |
| 1 | H4H14699P2 | 1 | 2 | 3 | 4 | 5 | 6 | 7 | 8 |
| 2 | H4H14700P2 | 17 | 18 | 19 | 20 | 21 | 22 | 23 | 24 |
| 3 | H4H14706P2 | 33 | 34 | 35 | 36 | 37 | 38 | 39 | 40 |
| 4 | H4H14708P2 | 49 | 50 | 51 | 52 | 53 | 54 | 55 | 56 |
| 5 | H4H14709P | 65 | 66 | 67 | 68 | 69 | 70 | 71 | 72 |
| 6 | H4H14728P | 81 | 82 | 83 | 84 | 85 | 86 | 87 | 88 |
| 7 | H4H14731P | 97 | 98 | 99 | 100 | 101 | 102 | 103 | 104 |
| 8 | H4H14732P2 | 113 | 114 | 115 | 116 | 117 | 118 | 119 | 120 |
| 9 | H4H14734P2 | 129 | 130 | 131 | 132 | 133 | 134 | 135 | 136 |
| 10 | H4H14757P | 137 | 138 | 139 | 140 | 141 | 142 | 143 | 144 |
| 11 | H4H14758P | 153 | 154 | 155 | 156 | 157 | 158 | 159 | 160 |
| 12 | H4H14760P2 | 169 | 170 | 171 | 172 | 173 | 174 | 175 | 176 |
| Antibody | | VK | | CDR1 | | CDR2 | | CDR3 | |
| # | Name | DNA | PEP | DNA | PEP | DNA | PEP | DNA | PEP |
| 1 | H4H14699P2 | 9 | 10 | 11 | 12 | 13 | 14 | 15 | 16 |
| 2 | H4H14700P2 | 25 | 26 | 27 | 28 | 29 | 30 | 31 | 32 |
| 3 | H4H14706P2 | 41 | 42 | 43 | 44 | 45 | 46 | 47 | 48 |
| 4 | H4H14708P2 | 57 | 58 | 59 | 60 | 61 | 62 | 63 | 64 |
| 5 | H4H14709P | 73 | 74 | 75 | 76 | 77 | 78 | 79 | 80 |
| 6 | H4H14728P | 89 | 90 | 91 | 92 | 93 | 94 | 95 | 96 |
| 7 | H4H14731P | 105 | 106 | 107 | 108 | 109 | 110 | 111 | 112 |
| 8 | H4H14732P2 | 121 | 122 | 123 | 124 | 125 | 126 | 127 | 128 |
| 9 | H4H14734P2 | 121 | 122 | 123 | 124 | 125 | 126 | 127 | 128 |
| 10 | H4H14757P | 145 | 146 | 147 | 148 | 149 | 150 | 151 | 152 |
| 11 | H4H14758P | 161 | 162 | 163 | 164 | 165 | 166 | 167 | 168 |
| 12 | H4H14760P2 | 121 | 122 | 123 | 124 | 125 | 126 | 127 | 128 |

*Numbers corresponding to V_{H}, CDR-H1, CDR-H2, CDR-H3, V_{L}, CDR-L1, CDR-L2 and CDR-L3 refer to SEQ ID NOs set forth herein.
“PEP” refers to an amino acid sequence;
“DNA” refers to a nucleotide sequence.

SEQ ID NO: 1
GAAGTGCAGCTGGTGGAGTCTGGGGGAGGCTTGGTGCAGCCTGGCAGGTCCCTGAGACTCTCCTGTGCGGCCTCTGGATT
CACCTTTGATGATTATGCCATACACTGGGTCCGGCAAGCTCCAGGGAAGGGCCTGGAGTGGGTCTCAGTTATCAGTTGGA
ATAGTGATATCATAGGCTATGCGGACTCTGTGAAGGGCCGATTCACCGTCTCCAGAGACAACGCCAAGAACTCCCTGTAT
CTGCAAATGAATAGTCTGAGAACTGAGGACACGGCCTTGTATTACTGTGCAAAAGGATATAACTGGAACTTCTTTGACTA
TTGGGGCCAGGGAACCCTGGTCACCGTCTCCTCA;
SEQ ID NO: 2
EVQLVESGGGLVQPGRSLRLSCAASGFTFDDYAIHWVRQAPGKGLEWVSVISWNSDIIGYADSVKGRFTVSRDKAKNSLY
LQMNSLRTEDTALYYCAKGYNWNFFDYWGQGTLVTVSS;
SEQ ID NO: 3
GGA TTC ACC TTT GAT GAT TAT GCC;
SEQ ID NO: 4
G F T F D D Y A;
SEQ ID NO: 5
ATC AGT TGG AAT AGT GAT ATC ATA;
SEQ ID NO: 6
I S W N S D I I;
SEQ ID NO: 7
GCA AAA GGA TAT AAC TGG AAC TTC TTT GAC TAT;
SEQ ID NO: 8
A K G Y N W N F F D Y;
SEQ ID NO: 9
GAAATTGTGTTGACGCAGTCTCCAGCCACCCTGTCTTTATCTCCAGGGGAAAGAGCCACCCTCTCCTGCAGGGCCAGTCA
GAGTGTTAGCAGCTACTTAGCCTGGTACCAACAGAAACCTGGCCAGGCTCCCAGGCTCCTCATCTATAATGCAGCAAACA
GGGCCACTGACATCCCAGCCAGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACTTTCGGCGGAGGGACCAAGGTGGAGATCAA
A;
SEQ ID NO: 10
EIVLTQSPATLSLSPGERATLSCRASQSVSSYLAWYQQKPGQAPRLLIYNAANRATDIPARFSGSGSGTDFTLTISSLEP
EDFAVYYCQQRSNWPLTFGGGTKVEIK;
SEQ ID NO: 11
CAG AGT GTT AGC AGC TAC;
SEQ ID NO: 12
Q S V S S Y;
SEQ ID NO: 13
AAT GCA GCA;
SEQ ID NO: 14
N A A;
SEQ ID NO: 15
CAG CAG CGT AGC AAC TGG CCT CTC ACT;
SEQ ID NO: 16
Q Q R S N W P L T;
SEQ ID NO: 17
GAAGTGCAGCTGGTGGAGTCTGGGGGAGGCTTGGTACAGCCTGGCAGGTCCCTGAGACTCTCCTGTGCAGCCTCTGGATT
CACCTTTGATGATTATGCCATGCACTGGGTCCGGCAAACTCCAGGGAAGGGCCTGGAGTGGGTCTCAGTTATTAGTTGGA
ATAGTGATGTCATAGCCTATTCGGACTCTGTGAAGGGCCGCTTCACCATTTCCAGAGACAACGCCAAGAACTCCCTGTAT
CTGCAAATGAACAGTCTGGGAACTGAGGACACGGCCTTATATTACTGTGCAAAAGGCCATAACTGGAACTTCTTTGACTA
TTGGGGCCAGGGAACCCTGGTCACCGTCTCCTCA;
SEQ ID NO: 18
EVQLVESGGGLVQPGRSLRLSCAASGFTFDDYAMHWVRQTPGKGLEWVSVISWNSDVIAYSDSVKGRFTISRDNAKNSLY
LQMNSLGTEDTALYYCAKGHNWNFFDYWGQGTLVTVSS;
SEQ ID NO: 19
GGA TTC ACC TTT GAT GAT TAT GCC;
SEQ ID NO: 20
G F T F D D Y A;
SEQ ID NO: 21
ATT AGT TGG AAT AGT GAT GTC ATA;
SEQ ID NO: 22
I S W N S D V I;
SEQ ID NO: 23
GCA AAA GGC CAT AAC TGG AAC TTC TTT GAC TAT;
SEQ ID NO: 24
A K G H N W N F F D Y;
SEQ ID NO: 25
GAAATTGTGTTGACACAGTCTCCAGCCACCCTGTCTTTGTCTCCAGGAGAAAGAGCCACCCTCTCCTGCAGGGCCAGTCA
GAGTGTTAGCAGCTACTTAGCCTGGTACCAACAGAAACCTGGCCAGGCTCCCAGGCTCCTCATCTATAATGTAGCCAACA
GGGCCACAGACATCCCAGCCAGGTTCAGTGGCAGTGGGTCTGGGACAGACTTCACTCTCACCATCAGCGGCCTAGAGCCT
GAAGATTTTGCAGTTTATTTCTGTCAGCAGCGTAGCAACTGGCCTCTCACTTTCGGCGGAGGGACCAAGGTGGAGATCAA
A;
SEQ ID NO: 26
EIVLTQSPATLSLSPGEPRATLSCRASQSVSSYLAWYQQKPGQAPRLLIYKVANRATDIPARFSGSGSGTDFTLTISGLEP
EDFAVYFCQQRSNWPLTFGGGTKVEIK;
SEQ ID NO: 27
CAG AGT GTT AGC AGC TAG;
SEQ ID NO: 28
Q S V S S Y;
SEQ ID NO: 29
AAT GTA GCC;
SEQ ID NO: 30
N V A;
SEQ ID NO: 31
CAG CAG CGT AGC AAC TGG CCT CTC ACT;
SEQ ID NO: 32
Q Q R S N W P L T;
SEQ ID NO: 33
GAAGTGCAGCTGGTGGAGTCTGGGGGAGGCTTGGTACAGCCTGGCAGGTCCCTGAGACTCTCCTGTACAGCCTCTGGATT
CACCTTTGATGATTATGCCATAGACTGGGTCCGGCAATCTCCAGGGAAGGGCCTGGAGTGGGTCTCAGTTATCAGTTGGA
ATAGTGATGTCATAGGCTATGCGGACTCTGTGAAGGGCCGATTCACCATCTCCAGAGACAACGCCAAGAACTCCCTGTAT
CTGCAGATGAATAGTCTGAGAGCTGAGGACACGGCCTTGTATTACTGTGCAAAAGGATATAACTGGAACTTCTTTGACTA
TTGGGGCCAGGGAACCCTGGTCACCGTCTCCTCA;
SEQ ID NO: 34
EVQLVESGGGLVQPGRSLRLSCTASGFTFDDYAIHWVRQSPGKGLEWVSVISWNSDVIGYADSVKGRFTISRDNAKNSLY
LQMNSLRAEDTALYYCAKGYNWNFFDYWGQGTLVTVSS;
SEQ ID NO: 35
GGA TTC ACC TTT GAT GAT TAT GCC;
SEQ ID NO: 36
G F T F D D Y A;
SEQ ID NO: 37
ATC AGT TGG AAT AGT GAT GTC ATA;
SEQ ID NO: 38
I S W N S D V I;
SEQ ID NO: 39
GCA AAA GGA TAT AAC TGG AAC TTC TTT GAC TAT;
SEQ ID NO: 40
A K G Y N W N F F D Y;
SEQ ID NO: 41
GAAATTGTGTTGACGCAGTCTCCAGCCACCCTGTCTTTATCTCCAGGGGAAAGAGCCACCCTCTCCTGCAGGGCCAGTCA
GAGTGTTAGCAGCTACTTAGCCTGGTACCAACAGAAACCTGGCCAGGCTCCCAGGCTCCTCATCTATAATGCAGCAAACA
GGGCCACTGACATCCCAGCCAGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACTTTCGGCGGAGGGACCAAGGTGGAGATCAA
A;
SEQ ID NO: 42
EIVLTQSPATLSLSPGERATLSCRASQSVSSYLAWYQQKPGQAPRLLIYNAANRATDIPARFSGSGSGTDFTLTISSLEP
EDFAVYYCQQRSNWPLTFGGGTKVEIK;
SEQ ID NO: 43
CAG AGT GTT AGC AGC TAC;
SEQ ID NO: 44
Q S V S S Y;
SEQ ID NO: 45
AAT GCA GCA;
SEQ ID NO: 46
K A A;
SEQ ID NO: 47
CAG CAG CGT AGC AAC TGG CCT CTC ACT;
SEQ ID NO: 48
Q Q R S N W P L T;
SEQ ID NO: 49
GAAGTGCAGCTGGTGGAGTCTGGGGGAGACTTGGTACAGCCTGGCAGGTCCCTGAGACTCTCCTGTGCAGCCTCTGGATT
CACCTTTGATGATTATGCCATGCACTGGGTGCGGCAAGCTCCAGGGAAGGGCCTGGAATGGGTCTCAGTTATTAGTTGGA
ATAGTGATGTCATAGCCTATTCGGACTCTGTGAAGGGCCGATTCACCATCTCCAGAGACAACGCCAAGAACTCCCTGTAT
CTGCAAATGAACAGTCTGAGAACTGAGGACACGGCCTTATATTACTGTACAAAAGGCCATAAGTGGAGCTTCTTTGACTA
TTGGGGCCAGGGAACCCTGGTCACCGTCTCCTCA;
SEQ ID NO: 50
EVQLVESGGDLVQPGRSLRLSCAASGFTFDDYAMHWVRQAPGKGLEWVSVISWNSDVIAYSDSVKGRFTISRDNAKMSLY
LQMNSLRTEDTALYYCTKGHKWSFFDYWGQGTLVTVSS;
SEQ ID NO: 51
GGA TTC ACC TTT GAT GAT TAT GCC;
SEQ ID NO: 52
G F T F D D Y A;
SEQ ID NO: 53
ATT AGT TGG AAT AGT GAT GTC ATA;
SEQ ID NO: 54
I S W N S D V I;
SEQ ID NO: 55
AGA AAA GGC CAT AAG TGG AGC TTC TTT GAC TAT;
SEQ ID NO: 56
T K G H K W S F F D Y;
SEQ ID NO: 57
GAAATTGTGTTGACACAGTCTCCAGCCACCCTGTCTTTGTCTCCAGGGGAAAGAGCCACCCTCTCCTGCAGGGCCAGTCA
GAGTATTAGCAGCTACTTAGCCTGGTACCAACAGAAACCTGGCCAGGCTCCCAGACTCCTCATCTTTAATGTAGCCAACA
GGGCCACTGACATCCCAGCCAGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACTTTCGGCGGAGGGACCAAGGTGGAGATCAA
A;
SEQ ID NO: 58
EIVLTQSPATLSLSPGERATLSCRASQSISSYLAWYQQKPGQAPRLLIFNVANRATDIPARFSGSGSGTDFTLTISSLEP
EDFAVYYCQQRSNWPLTFGGGTKVEIK;
SEQ ID NO: 59
CAG AGT ATT AGC AGC TAC;
SEQ ID NO: 60
Q S I S S Y;
SEQ ID NO: 61
AAT GTA GCC;
SEQ ID NO: 62
N V A;
SEQ ID NO: 63
CAG CAG CGT AGC AAC TGG CCT CTC ACT;
SEQ ID NO: 64
Q Q R S N W P L T;
SEQ ID NO: 65
GAGGTGCAGCTGGTGGAGTCTGGGGGAGGCTTGGTTCAGCCTGGGGGGTCCCTGAGACTCTCCTGCGCAGCCTCTGGATT
CACCTTTAGCGACTATGCCATGAGCTGGGTCCGCCAGGCTCCGGGGAAGGGGCTGGAGTGGGTCTCAGGTATTAGTGGAA
ATGGTGGTGACACATACTACGGAGACTTCGTGAAGGGCCGGTTCACCATCTCCAGAGACAATTCCAAGAACACGCTGTAT
CTGCAAATGAACAGCCTGAGAGGCGAGGACACGGCCGCATATTTCTGTGTGATAGATCTTGACTATTGGGGTCAGGGAAC
CCTGGTCACCGTCTCCTCA;
SEQ ID NO: 66
EVQLVESGGGLVQPGGSLRLSCAASGFTFSDYAMSWVRQAPGKGLEWVSGISGNGGDTYYGDFVKGRFTISRDNSKNTLY
LQMNSLRGEDTAAYFCVIDLDYWGQGTLVTVSS;
SEQ ID NO: 67
GGA TTC ACC TTT AGC GAC TAT GCC;
SEQ ID NO: 68
G F T F S D A;
SEQ ID NO: 69
ATT AGT GGA AAT GGT GGT GAC ACA;
SEQ ID NO: 70
I S G N G G D T;
SEQ ID NO: 71
GTG ATA GAT CTT GAC TAT;
SEQ ID NO: 72
V I D L D Y;
SEQ ID NO: 73
GACATCCAGATGACCCAGTCTCCTTCCACCCTGTCTGCATCTGAAGGAGACAGAGTCACCATCACTTGCCGGGCCAGTCA
GAGTATTAGTAGCTGGTTGGCCTGGTATCAACAGAAACCAGGAAAAGCCCCTAGGCTCCTGATCTATAAGGCGTCTATTT
TAGGAGATGGGGTCCCATCAAGGTTCAGCGGCAGTGGATCTGGGACAGAATTCACTCTCACCATCAGCAGCCTGCAGCCT
GATGATTTTGCTACTTATTACTGCCACCAGTATAATAGTTATTTGTGGACGTTCGGCCAAGGGACCAAGGTGGAAATCAA
A;
SEQ ID NO: 74
DIQMTQSPSTLSASEGDRVTITCRASQSISSWLAWYQQKPGKAPRLLIYKASILGDGVPSRFSGSGSGTEFTLTISSLQP
DDFATYYCHQYNSYLWTFGQGTKVEIK;
SEQ ID NO: 75
CAG AGT ATT AGT AGC TGG;
SEQ ID NO: 76
Q S I S S W;
SEQ ID NO: 77
AAG GCG TCT;
SEQ ID NO: 78
K A S;
SEQ ID NO: 79
CAC CAG TAT AAT AGT TAT TTG TGG ACG;
SEQ ID NO: 80
H Q Y N S Y L W T;
SEQ ID NO: 81
CAGGTGCAGCTGCAGGAGTCGGGCCCAGGACTGGTGAAGCCTTCACAGACCCTGTCCCTCACCTGCACTGTCTCTGGTGG
CTCCATCAGCAGTGCTGATTACTATTGGAGCTGGATCCGCCAGCACCCAGGGAAGGGCCTGGAGTGGATTGGATCCATCT
ATTATACTGGGAGTACTTACTACAACCCGTCCCTCAAGAGTCGACTTACCATATCAATAGACACGTCTGAGAACCAGTTC
TCTTTGAAACTGACCTCTCTGACTGCCGCGGACACGGCCGTGTATTACTGTGCGAGCGAGGAGGCTAACTGGGGATCCGA
CTTTGACTCCTGGGGCCAGGGAACCCTGGTCACCGTCTCCTCA;
SEQ ID NO: 82
QVQLQESGPGLVKPSQTLSLTCTVSGGSISSADYYWSWIRQKPGKGLEWIGSIYYTGSTYYNPSLKSRLTISIDTSENQF
SLKLTSLTAADTAVYYCAEEANWGSHFDSWGQGTLVTVSS;
SEQ ID NO: 83
GGT GGC TCC ATC AGC AGT GCT GAT TAC TAT;
SEQ ID NO: 84
G G S I S S A D Y Y;
SEQ ID NO: 85
ATC TAT TAT ACT GGG AGT ACT;
SEQ ID NO: 86
I Y Y T G S T;
SEQ ID NO: 87
GCG AGC GAG GAG GCT AAC TGG GGA TCC CAC TTT GAC TCC;
SEQ ID NO: 88
A S E E A N W G S H F D S;
SEQ ID NO: 89
GACATCCAGATGACCCAGTCTCCATCCTCCCTGTCTGCATCTGTAGGAGACAGAGTCACCATCACTTGCCGGGCAAGTCA
GAGCATTGACAACTTTTTAAATTGGTATCAGCAGAAACCAGGGAAAGCCCCTAAGCTCCTGATCTATGCTGCATCCAGTT
TGCAAAGTGGGGTCCCATCAAGGTTCAGTGGCAGTGGATCTGGGACAGATTTCACTCTCACCATCAGCAGTCTGCAACCT
GAAGATTTTGCATCTTACTACTGTCAAGATAGTCACAGTGCCCATCCGATCACCTTCGGCCAAGGGACACGACTGGAGAT
TAAA;
SEQ ID NO: 90
DIQMTQSPSSLSASVGDRVTITCRASQSIDNFLNWYQQKPGKAPKLLIYAASSLQSGVPSRFSGSGSGTDFTLTISSLQP
EDFASYYCQHSHSAHPITFGQGTRLEIK;
SEQ ID NO: 91
CAG AGC ATT GAC AAC TTT;
SEQ ID NO: 92
Q S I D N F;
SEQ ID NO: 93
GCT GCA TCC;
SEQ ID NO: 94
A A S;
SEQ ID NO: 95
CAA CAT AGT CAC AGT GCC CAT CCG ATC ACC;
SEQ ID NO: 96
Q H S K S A H P I T;
SEQ ID NO: 97
CAGCTGCAGCTGCAGGAGTCGGGCCCAGGACTGGTGAAGCCTTCGGAGACCCTGTCCCTCACCTGCACTGTCTCTGGTGG
CTCCATCAGCAGTAGTAATTACTACTGGGGCTGGATCCGCCAGCCCCCAGGGAAGAGACTGGAGTGGATTGGGAGTATCT
ATTATAGTGGGAGCACCTACTACAACCCGTCCCTCAAGACTCGAGTCACCATATCCGTAGACACGTCCAAGAATCAGTTC
TCCCTGAAGCTGACCTCTGTGACCGCCGCAGACACGGCTGTGTATTACTGTGCGAGAGAGGAAGCAGCAGCTTTGACGCA
CTTTGACTTCTGGGGCCAGGGAACCCTGGTCACCGTCTCCTCA;
SEQ ID NO: 98
QLQLQESGPGLVKPSETLSLTCTVSGGSISSSNYYWGWIRQPPGKRLEWIGSIYYSGSTYYNPSLKTRVTISVDTSKNQF
SLKLTSVTAADTAVYYCAREEAAALTHFDFWGQGTLVTVSS;
SEQ ID NO: 99
GGT GGC TCC ATC AGC AGT AGT AAT TAC TAG;
SEQ ID NO: 100
G G S I S S S N Y Y;
SEQ ID NO: 101
ATC TAT TAT AGT GGG AGC ACC;
SEQ ID NO: 102
I Y Y S G S T;
SEQ ID NO: 103
GCG AGA GAG GAA GCA GCA GCT TTG ACG CAC TTT GAC TTC;
SEQ ID NO: 104
A R E E A A A L T H F D F;
SEQ ID NO: 105
GACATCCAGATGACCCAGTCTCCATCCTCCCTGTCTGCATCTGTAGGAGACAGAGTCACCATCACTTGCCGGGCAAGTCA
GAGCATTAGCAACTATTTAAATTGGTATCAGCAGAAACCAGGGAAAGCCCCTAAGCTCCTGATCTTTGCTGCATCCAGTT
TACAAAGTGGGGTCCCATCAAGGTTCAGTGGCAGTGGATCTGGGACAGATTTCACTCTCACCATCAGCAGTCTGCAACCT
GAAGATTTTGCAACTTACTACTGTCAACATAGTCACAGTTCCCATCCGATCACCTTCGGCCAAGGGACACGACTGGAGAT
TAAA;
SEQ ID NO: 106
DIQMTQSPSSLSASVGDRVTITCRASQSISNYLNWYQQKPGKAPKLLIFAASSLQSGVPSRFSGSGSGTDFTLTISSLQP
EDFATYYCQHSHSSHPITFGQGTRLEIK;
SEQ ID NO: 107
CAG AGC ATT AGC AAC TAT;
SEQ ID NO: 108
Q S I S N Y;
SEQ ID NO: 109
GCT GCA TCC;
SEQ ID NO: 110
A A S;
SEQ ID NO: 111
CAA CAT AGT CAC AGT TCC CAT CCG ATC ACC;
SEQ ID NO: 112
Q H S H S S H P I T;
SEQ ID NO: 113
GAAGTGCAGCTGGTGGAGTCTGGGGGAGGCTTGGTACAGCCTGGCAGGTCCCTGAGACTCTCCTGTGCAGCCTCTGGATT
CACCTTTGATGATTATGCCATGCACTGGGTCCGGCAAGCTCCAGGGAAGGGCCTGGAGTGGGTCTCAGGTATTAATTGGG
CTGGTTATAACATAGACTATGCGGACTCTGTGAAGGGCCGATTCACCATCTCCAGAGACAACGCCAAGAACTCCCTGTAT
CTGCAAATGAACAGTCTGAGAGCTGAGGACACGGCCTTGTATTACTGTGCAAAAGATATGCGTGGATTCAGTTATGGTTT
CCCCTTTGACTACTGGGGCCAGGGAACCCTGGTCACCGTCTCCTCA;
SEQ ID NO: 114
EVQLVESGGGLVQPGRSLRLSCAASGFTFDDYAMHWVRQAPGKGLEWVSGINWAGYNIDYADSVKGRFTISRDNAKNSLY
LQMNSLRAEDTALYYCAKDMRGFSYGFPFDYWGQGTLVTVSS;
SEQ ID NO: 115
GGA TTC ACC TTT GAT GAT TAT GCC;
SEQ ID NO: 116
G F T F D D Y A;
SEQ ID NO: 117
ATT AAT TGG GCT GGT TAT AAC ATA;
SEQ ID NO: 118
I N W A G Y N I;
SEQ ID NO: 119
GCA AAA GAT ATG CGT GGA TTC AGT TAT GGT TTC CCC TTT GAC TAC;
SEQ ID NO: 120
A K D M R G F S Y G F P F D Y;
SEQ ID NO: 121
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA;
SEQ ID NO: 122
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK;
SEQ ID NO: 123
CAG AGC ATT AGC AGC TAT;
SEQ ID NO: 124
Q S I S S Y;
SEQ ID NO: 125
GCT GCA TCC;
SEQ ID NO: 126
A A S;
SEQ ID NO: 127
CAA CAG AGT TAG AGT ACC CCT CCG ATC ACC;
SEQ ID NO: 128
Q Q S Y S T P P I T;
SEQ ID NO: 129
GAGGTGCAGCTGGTGGAGTCTGGGGGAGGCTTGGTAAAGCCGGGGGGGTCCCTTAGACTCTCCTGTGCAGCCTCTGGATT
TATTTTCAGTAACGCCTGGATGAACTGGGTCCGCCAGGCTCCAGGGAAGGGACTGGCGTGGGTTGGCCGTATTAAAACCG
AAACTGATGGTGGGACAACAGACTACGCTGCACCCGTAAAAGGCAGATTCACCATCTCAAGAGATGACTCAAAAAACACG
CTGTATCTGCAAATGAACAGCGTGAAAACCGAGGACACAGCCGTGTATTACTGTACAGGGGGATACAGCTATGGTGACGA
TAGCAGCAGCTGGAACGAGGGCTACTACTACTACGGTATGGACGTCTGGGGCCAAGGGACCACGGTCACCGTCTCCTCA;
SEQ ID NO: 130
EVQLVESGGGLVKPGGSLRLSCAASGFIFSNAWMNWVRQAPGKGLAWVGRIKTETDGGTTDYAAPVKGRFTISRDDSKNT
LYLQMNSVKTEDTAVYYCTGGYSYGDDSSSWNEGYYYYGMDVWGQGTTVTVSS;
SEQ ID NO: 131
GGA TTT ATT TTC AGT AAC GCC TGG;
SEQ ID NO: 132
G F I F S N A W;
SEQ ID NO: 133
ATT AAA ACC GAA ACT GAT GGT GGG ACA ACA;
SEQ ID NO: 134
I K T E T D G G T T;
SEQ ID NO: 135
ACA GGG GGA TAC AGC TAT GGT GAC GAT AGC AGC AGC TGG AAC GAG GGC TAG TAC TAG TAC
GGT ATG GAC GTC;
SEQ ID NO: 136
T G G Y S Y G D D S S S W N E G Y Y Y Y
G M D V;
SEQ ID NO: 137
GAAGTGGAGCTGGTGGAGTCTGGGGGAGGCTTGGTACAGCCTGGCAGGTCCCTGAGACTCTCCTGTACAGCCTCTGGATT
CACCTTTGATGATTATGCCATGCACTGGGTCCGGCAAGCTCCAGGGAAGGGCCTGGAGTGGGTCTCAGGTATTCGTTGGA
ATGGTGGTAGTATAGGCTATGTGGAGTCTGTGAAGGGCCGATTCACCATCTCCAGAGAGAACGCCAAGAAGTCCGTGCAT
CTGCAAATGAACAGTCTAAAAACTGAGGACACGGCCTTGTATTACTGTGCAAAAGATATAGGCGATATTTTGACTGGTTT
TTATGGAGAATACGGAATGGACGTCTGGGGCCAAGGGACCACGGTCACCGTCTCCTCA;
SEQ ID NO: 138
EVQLVESGGGLVQPGRSLRLSCTASGFTFDDYAMHWVRQAPGKGLEWVSGIRWNGGSIGYVDSVKGRFTISRDNAKKSLH
LQMNSLKTEDTALYYCAKDIGDILTGFYGEYGMDVWGQGTTVTVSS;
SEQ ID NO: 139
GGA TTC ACC TTT GAT GAT TAT GCC;
SEQ ID NO: 140
G F T F D D Y A;
SEQ ID NO: 141
ATT CGT TGG AAT GGT GGT AGT ATA;
SEQ ID NO: 142
I R W N G G S I;
SEQ ID NO: 143
GCA AAA GAT ATA GGC GAT ATT TTG ACT GGT TTT TAT GGA GAA TAC GGA ATG GAC GTC;
SEQ ID NO: 144
A K D I G D I L T G F Y G E Y G M D V;
SEQ ID NO: 145
GACATCCAGATGACCCAGTCTCCATCCTCCCTGTCTGCATCTGAAGGAGACAGAGTCACCATCACTTGCCGGGCAAGTCA
GAGCATTAGCAGCTATTTAAATTGGTATCAGCAGAAAGCAGGGAAAGCCCCTAACCTCCTGATCTATGCTGCATCCAGTT
TGCAAAGTGGGGTCCCATCAAGGTTCAGTGGCAGTGGATCTGGGACAGAGTACACTCTCACCATCAGCAGTCTGCAACCT
GAAGATTTTGCAACTTACTACTGTCAACAGAGTTACATTATCCCGTACACTTTTGGCCAGGGGACCAAGCTGGAGATCAA
A;
SEQ ID NO: 146
DIQMTQSPSSLSASEGDRVTITCRASQSISSYLNWYQQKAGKAPNLLIYAASSLQSGVPSRFSGSGSGTEYTLTISSLQP
EDFATYYCQQSYIIPYTFGQGTKLETK;
SEQ ID NO: 147
CAG AGC ATT AGC AGC TAT;
SEQ ID NO: 148
Q S I S S Y;
SEQ ID NO: 149
GCT GCA TCC;
SEQ ID NO: 150
A A S;
SEQ ID NO: 151
CAA CAG AGT TAG ATT ATG CCG TAG ACT;
SEQ ID NO: 152
Q Q S Y I I P Y T;
SEQ ID NO: 153
GAAGTGCAGCTGGTGGAGTCTGGGGGAGGGTTGGTACAGCCTGGCAGGTCCCTGAGACTCTCCTGTGCAGCCTCTGGATT
CACCTTTGATGATTATGCCATGCACTGGGTCCGGCAAGCTCCAGGGAAGGGCCTGGAGTGGGTCTCAAGTGTTAGGTGGA
ATGGTGGTATTATAGGCTATGCGGACTCTGTGAAGGGCCGATTCACCATCTCCAGAGACAACGCCAAGAACTCCCTGTAT
CTGCAAATGAACAGTCTGAGACCTGAGGACACGGCCCTCTATTACTGTGCAAAAGATATAGGCGATGTTTTGACTGGTTA
TTATGGAGAATACGGTATGGACGTCTGGGGCCAAGGGACCACGGTCACCGTCTCCTCA;
SEQ ID NO: 154
EVQLVESGGGLVQPGRSLRLSCAASGFTFDDYAMHWVRQAPGKGLEWVSSVRWKGGIIGYADSVKGRFTISRDNAKNSLY
LQMNSLRPEDTALYYCAKDIGDVLTGYYGEYGMDVWGQGTTVTVSS;
SEQ ID NO: 155
GGA TTC ACC TTT GAT GAT TAT GCC;
SEQ ID NO: 156
G F T F D D Y A;
SEQ ID NO: 157
GTT AGG TGG AAT GGT GGT ATT ATA;
SEQIDNO: 158
V R W N G G I I;
SEQ ID NO: 159
GCA AAA GAT ATA GGC GAT GTT TTG ACT GGT TAT TAT GGA GAA TAG GGT ATG GAC GTC;
SEQ ID NO: 160
A K D I G D V L T G Y Y G E Y G M D V;
SEQ ID NO: 161
GACATCCAGATGACCCAGTCTCCATCCTCCCTGTCTGCATCTGTGGGAGACAGAGTCACCATCGCTTGCCGGGCAAGTCA
GAGCATTACCACCTATTTAAATTGGTATCAGCAGAAACCAGGGAAAGCCCCTAAACTCCTGATCTATGCTGCATCCAGTT
TGCAAAGTGGGGTCCCATCAAGGTTCAGTGGCAGTGGATCTGGGACAGATTTCACTCTCACCATCAGTAGTCTGCAACCT
GAAGATTTTGCAACTTACTACTGTCAACAGAGTTACATTTCCCCGTACACTTTTGGCCAGGGGACCAAGCTGGAGATCAAA;
SEQ ID NO: 162
DIQMTQSPSSLSASVGDRVTIACRASQSITTYLNWYQQKPGKAPKLLIYAASSLQSGVPSRFSGSGSGTDFTLTISSLQP
EDFATYYCQQSYISPYTFGQGTKLEIK;
SEQ ID NO: 163
CAG AGC ATT ACC ACC TAT;
SEQ ID NO: 164
Q S I T T Y;
SEQ ID NO: 165
GCT GCA TCC;
SEQ ID NO: 166
A A S;
SEQ ID NO: 167
CAA CAG AGT TAC ATT TCC CCG TAC ACT;
SEQ ID NO: 168
Q Q S Y I S P Y T;
SEQ ID NO: 169
CAGGTGCAGCTGGTGGAGTCTGGGGGAGGCGTGGTCCAGCCTGGGAAGTCCCTGAGACTCTCCTGTGCAGCCTCTGGATT
CACCTTCAGTAATTATGGCATACACTGGGTCCGCCAGGCTCCAGGCAAGGGGCTGGAGTGGGTGGCGATTATATTATATG
ATGGAAGTAATCAACACTATGCAGATTCCGTGAAGGGCCGATTCACCATTTCCAGAGACAATTCCAAAAACACGCTGTAT
CTTCAAATGAACAACCTGAGAGCTGAGGACACGGCCGTTTATTACTGTGCGAGAGATCTTGATCTTTGGAGTGGTTATTA
TACAAACGGGGACGGTATGGACGTCTGGGGCCAAGGGACCACGGTCACCGTCTCCTCA;
SEQ ID NO: 170
QVQLVESGGGVVQPGKSLRLSCAASGFTFSNYGIHWVRQAPGKGLEWVAIILYDGSNQHYADSVKGRFTISRDKSKNTLY
LQMNNLRAEDTAVYYCARDLDLWSGYYTNGDGMDVWGQGTTVTVSS;
SEQ ID NO: 171
GGA TTC ACC TTC AGT AAT TAT GGC;
SEQ ID NO: 172
G F T F S N Y G;
SEQ ID NO: 173
ATA TTA TAT GAT GGA AGT AAT CAA;
SEQ ID NO: 174
I L Y D G S N Q;
SEQ ID NO: 175
GCG AGA GAT CTT GAT CTT TGG AGT GGT TAT TAT ACA AAC GGG GAC GGT ATG GAG GTC;
SEQ ID NO: 176
A R D L D L W S G Y Y T N G D G M D V;

The amino acid and nucleotide sequences of heavy and light chain immunoglobulins, including constant domains, of antigen-binding proteins of the present invention are set forth below:

H4H14699P2
Heavy chain DNA
(SEQ ID NO: 179)
GAAGTGCAGCTGGTGGAGTCTGGGGGAGGCTTGGTGCAGCCTGGCAGGTCCCTGAGACTCTCCTGTGCGGCCTCTGGATT
CACCTTTGATGATTATGCCATACACTGGGTCCGGCAAGCTCCAGGGAAGGGCCTGGAGTGGGTCTCAGTTATCAGTTGGA
ATAGTGATATCATAGGCTATGCGGACTCTGTGAAGGGCCGATTCACCGTCTCCAGAGACAACGCCAAGAACTCCCTGTAT
CTGCAAATGAATAGTCTGAGAACTGAGGACACGGCCTTGTATTACTGTGCAAAAGGATATAACTGGAACTTCTTTGACTA
TTGGGGCCAGGGAACCCTGGTCACCGTCTCCTCAGCCTCCACCAAGGGCCCATCGGTCTTCCCCCTGGCGCCCTGCTCCA
GGAGCACCTCCGAGAGCACAGCCGCCCTGGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTCAGGACTCTACTCCCTCAGCAGCGTGGT
GACCGTGCCCTCCAGCAGCTTGGGCACGAAGACCTACACCTGCAACGTAGATCACAAGCCCAGCAACACCAAGGTGGACA
AGAGAGTTGAGTCCAAATATGGTCCCCCATGCCCACCCTGCCCAGCACCTGAGTTCCTGGGGGGACCATCAGTCTTCCTG
TTCCCCCCAAAACCCAAGGACACTCTCATGATCTCCCGGACCCCTGAGGTCACGTGCGTGGTGGTGGACGTGAGCCAGGA
AGACCCCGAGGTCCAGTTCAACTGGTACGTGGATGGCGTGGAGGTGCATAATGCCAAGACAAAGCCGCGGGAGGAGCAGT
TCAACAGCACGTACCGTGTGGTCAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGCAGCCCCGAGAGCCACAGGTGTA
CACCCTGCCCCCATCCCAGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCCGTGCTGGACTCCGACGGC
TCCTTCTTCCTCTACAGCAGGCTCACCGTGGACAAGAGCAGGTGGCAGGAGGGGAATGTCTTCTCATGCTCCGTGATGCA
TGAGGCTCTGCACAACCACTACACACAGAAGTCCCTCTCCCTGTCTCTGGGTAAATGA
Heavy chain polypeptide
(SEQ ID NO: 180)
EVQLVESGGGLVQPGRSLRLSCAASGFTFDDYAIHWVRQAPGKGLEWVSVISWDSDIIGYADSVKGRFTVSRDNAKNSLY
LQMNSLRTEDTALYYCAKGYNWNFFDYWGQGTLVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Light chain DNA
(SEQ ID NO: 181)
GAAATTGTGTTGACGCAGTCTCCAGCCACCCTGTCTTTATCTCCAGGGGAAAGAGCCACCCTCTCCTGCAGGGCCAGTCA
GAGTGTTAGCAGCTACTTAGCCTGGTACCAACAGAAACCTGGCCAGGCTCCCAGGCTCCTCATCTATAATGCAGCAAACA
GGGCCACTGACATCCCAGCCAGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACTTTCGGCGGAGGGACCAAGGTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 182)
EIVLTQSPATLSLSPGERATLSCRASQSVSSYLAWYQQKPGQAPRLLIYNAANRATDIPARFSGSGSGTDFTLTISSLEP
EDFAVYYCQQRSNWPLTFGGGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H14700P2
Heavy chain DNA
(SEQ ID NO: 183)
GAAGTGCAGCTGGTGGAGTCTGGGGGAGGCTTGGTACAGCCTGGCAGGTCCCTGAGACTCTCCTGTGCAGCCTCTGGATT
CACCTTTGATGATTATGCCATGCACTGGGTCCGGCAAACTCCAGGGAAGGGCCTGGAGTGGGTCTCAGTTATTAGTTGGA
ATAGTGATGTCATAGCCTATTCGGACTCTGTGAAGGGCCGCTTCACCATTTCCAGAGACAACGCCAAGAACTCCCTGTAT
CTGCAAATGAACAGTCTGGGAACTGAGGACACGGCCTTATATTACTGTGCAAAAGGCCATAACTGGAACTTCTTTGACTA
TTGGGGCCAGGGAACCCTGGTCACCGTCTCCTCAGCCTCCACCAAGGGCCCATCGGTCTTCCCCCTGGCGCCCTGCTCCA
GGAGCACCTCCGAGAGCACAGCCGCCCTGGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTCAGGACTCTACTCCCTCAGCAGCGTGGT
GACCGTGCCCTCCAGCAGCTTGGGCACGAAGACCTACACCTGCAACGTAGATCACAAGCCCAGCAACACCAAGGTGGACA
AGAGAGTTGAGTCCAAATATGGTCCCCCATGCCCACCCTGCCCAGCACCTGAGTTCCTGGGGGGACCATCAGTCTTCCTG
TTCCCCCCAAAACCCAAGGACACTCTCATGATCTCCCGGACCCCTGAGGTCACGTGCGTGGTGGTGGACGTGAGCCAGGA
AGACCCCGAGGTCCAGTTCAACTGGTACGTGGATGGCGTGGAGGTGCATAATGCCAAGACAAAGCCGCGGGAGGAGCAGT
TCAACAGCACGTACCGTGTGGTCAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGCAGCCCCGAGAGCCACAGGTGTA
CACCCTGCCCCCATCCCAGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCCGTGCTGGACTCCGACGGC
TCCTTCTTCCTCTACAGCAGGCTCACCGTGGACAAGAGCAGGTGGCAGGAGGGGAATGTCTTCTCATGCTCCGTGATGCA
TGAGGCTCTGCACAACCACTACACACAGAAGTCCCTCTCCCTGTCTCTGGGTAAATGA
Heavy chain polypeptide
(SEQ ID NO: 184)
EVQLVESGGGLVQPGRSLRLSCAASGFTFDDYAMHWVRQTPGKGLEWVSVISWNSDVIAYSDSVKGRFTISRDNAKNSLY
LQMNSLGTEDTALYYCAKGHNWNFFDYWGQGTLVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Light chain DNA
(SEQ ID NO: 185)
GAAATTGTGTTGACACAGTCTCCAGCCACCCTGTCTTTGTCTCCAGGAGAAAGAGCCACCCTCTCCTGCAGGGCCAGTCA
GAGTGTTAGCAGCTACTTAGCCTGGTACCAACAGAAACCTGGCCAGGCTCCCAGGCTCCTCATCTATAATGTAGCCAACA
GGGCCACAGACATCCCAGCCAGGTTCAGTGGCAGTGGGTCTGGGACAGACTTCACTCTCACCATCAGCGGCCTAGAGCCT
GAAGATTTTGCAGTTTATTTCTGTCAGCAGCGTAGCAACTGGCCTCTCACTTTCGGCGGAGGGACCAAGGTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 186)
EIVLTQSPATLSLSPGERATLSCRASQSVSSYLAWYQQKPGQAPRLLIYNVANRATDIPARFSGSGSGTDFTLTISGLEP
EDFAVYFCQQRSNWPLTFGGGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H14706P2
Heavy chain DNA
(SEQ ID NO: 187)
GAAGTGCAGCTGGTGGAGTCTGGGGGAGGCTTGGTACAGCCTGGCAGGTCCCTGAGACTCTCCTGTACAGCCTCTGGATT
CACCTTTGATGATTATGCCATACACTGGGTCCGGCAATCTCCAGGGAAGGGCCTGGAGTGGGTCTCAGTTATCAGTTGGA
ATAGTGATGTCATAGGCTATGCGGACTCTGTGAAGGGCCGATTCACCATCTCCAGAGACAACGCCAAGAACTCCCTGTAT
CTGCAGATGAATAGTCTGAGAGCTGAGGACACGGCCTTGTATTACTGTGCAAAAGGATATAACTGGAACTTCTTTGACTA
TTGGGGCCAGGGAACCCTGGTCACCGTCTCCTCAGCCTCCACCAAGGGCCCATCGGTCTTCCCCCTGGCGCCCTGCTCCA
GGAGCACCTCCGAGAGCACAGCCGCCCTGGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTCAGGACTCTACTCCCTCAGCAGCGTGGT
GACCGTGCCCTCCAGCAGCTTGGGCACGAAGACCTACACCTGCAACGTAGATCACAAGCCCAGCAACACCAAGGTGGACA
AGAGAGTTGAGTCCAAATATGGTCCCCCATGCCCACCCTGCCCAGCACCTGAGTTCCTGGGGGGACCATCAGTCTTCCTG
TTCCCCCCAAAACCCAAGGACACTCTCATGATCTCCCGGACCCCTGAGGTCACGTGCGTGGTGGTGGACGTGAGCCAGGA
AGACCCCGAGGTCCAGTTCAACTGGTACGTGGATGGCGTGGAGGTGCATAATGCCAAGACAAAGCCGCGGGAGGAGCAGT
TCAACAGCACGTACCGTGTGGTCAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGCAGCCCCGAGAGCCACAGGTGTA
CACCCTGCCCCCATCCCAGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCCGTGCTGGACTCCGACGGC
TCCTTCTTCCTCTACAGCAGGCTCACCGTGGACAAGAGCAGGTGGCAGGAGGGGAATGTCTTCTCATGCTCCGTGATGCA
TGAGGCTCTGCACAACCACTACACACAGAAGTCCCTCTCCCTGTCTCTGGGTAAATGA
Heavy chain polypeptide
(SEQ ID NO: 188)
EVQLVESGGGLVQPGRSLRLSCTASGFTFDDYAIHWVRQSPGKGLEWVSVISWNSDVIGYADSVKGRFTISRDNAKNSLY
LQMNSLRAEDTALYYCAKGYNWNFFDYWGQGTLVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Light chain DNA
(SEQ ID NO: 189)
GAAATTGTGTTGACGCAGTCTCCAGCCACCCTGTCTTTATCTCCAGGGGAAAGAGCCACCCTCTCCTGCAGGGCCAGTCA
GAGTGTTAGCAGCTACTTAGCCTGGTACCAACAGAAACCTGGCCAGGCTCCCAGGCTCCTCATCTATAATGCAGCAAACA
GGGCCACTGACATCCCAGCCAGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACTTTCGGCGGAGGGACCAAGGTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 190)
EIVLTQSPATLSLSPGERATLSCRASQSVSSYLAWYQQKPGQAPRLLIYNAANRATDIPARFSGSGSGTDFTLTISSLEP
EDFAVYYCQQRSNWPLTFGGGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H14708P2
Heavy chain DNA
(SEQ ID NO: 191)
GAAGTGCAGCTGGTGGAGTCTGGGGGAGACTTGGTACAGCCTGGCAGGTCCCTGAGACTCTCCTGTGCAGCCTCTGGATT
CACCTTTGATGATTATGCCATGCACTGGGTCCGGCAAGCTCCAGGGAAGGGCCTGGAATGGGTCTCAGTTATTAGTTGGA
ATAGTGATGTCATAGCCTATTCGGACTCTGTGAAGGGCCGATTCACCATCTCCAGAGACAACGCCAAGAACTCCCTGTAT
CTGCAAATGAACAGTCTGAGAACTGAGGACACGGCCTTATATTACTGTACAAAAGGCCATAAGTGGAGCTTCTTTGACTA
TTGGGGCCAGGGAACCCTGGTCACCGTCTCCTCAGCCTCCACCAAGGGCCCATCGGTCTTCCCCCTGGCGCCCTGCTCCA
GGAGCACCTCCGAGAGCACAGCCGCCCTGGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTCAGGACTCTACTCCCTCAGCAGCGTGGT
GACCGTGCCCTCCAGCAGCTTGGGCACGAAGACCTACACCTGCAACGTAGATCACAAGCCCAGCAACACCAAGGTGGACA
AGAGAGTTGAGTCCAAATATGGTCCCCCATGCCCACCCTGCCCAGCACCTGAGTTCCTGGGGGGACCATCAGTCTTCCTG
TTCCCCCCAAAACCCAAGGACACTCTCATGATCTCCCGGACCCCTGAGGTCACGTGCGTGGTGGTGGACGTGAGCCAGGA
AGACCCCGAGGTCCAGTTCAACTGGTACGTGGATGGCGTGGAGGTGCATAATGCCAAGACAAAGCCGCGGGAGGAGCAGT
TCAACAGCACGTACCGTGTGGTCAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGCAGCCCCGAGAGCCACAGGTGTA
CACCCTGCCCCCATCCCAGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCCGTGCTGGACTCCGACGGC
TCCTTCTTCCTCTACAGCAGGCTCACCGTGGACAAGAGCAGGTGGCAGGAGGGGAATGTCTTCTCATGCTCCGTGATGCA
TGAGGCTCTGCACAACCACTACACACAGAAGTCCCTCTCCCTGTCTCTGGGTAAATGA
Heavy chain polypeptide
(SEQ ID NO: 192)
EVQLVESGGDLVQPGRSLRLSCAASGFTFDDYAMHWVRQAPGKGLEWVSVISWNSDVIAYSDSVKGRFTISRDNAKNSLY
LQMNSLRTEDTALYYCTKGHKWSFFDYWGQGTLVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Light chain DNA
(SEQ ID NO: 193)
GAAATTGTGTTGACACAGTCTCCAGCCACCCTGTCTTTGTCTCCAGGGGAAAGAGCCACCCTCTCCTGCAGGGCCAGTCA
GAGTATTAGCAGCTACTTAGCCTGGTACCAACAGAAACCTGGCCAGGCTCCCAGACTCCTCATCTTTAATGTAGCCAACA
GGGCCACTGACATCCCAGCCAGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACTTTCGGCGGAGGGACCAAGGTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 194)
EIVLTQSPATLSLSPGERATLSCRASQSISSYLAWYQQKPGQAPRLLIFNVANRATDIPARFSGSGSGTDFTLTISSLEP
EDFAVYYCQQRSNWPLTFGGGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H14709P
Heavy chain DNA
(SEQ ID NO: 195)
GAGGTGCAGCTGGTGGAGTCTGGGGGAGGCTTGGTTCAGCCTGGGGGGTCCCTGAGACTCTCCTGCGCAGCCTCTGGATT
CACCTTTAGCGACTATGCCATGAGCTGGGTCCGCCAGGCTCCGGGGAAGGGGCTGGAGTGGGTCTCAGGTATTAGTGGAA
ATGGTGGTGACACATACTACGGAGACTTCGTGAAGGGCCGGTTCACCATCTCCAGAGACAATTCCAAGAACACGCTGTAT
CTGCAAATGAACAGCCTGAGAGGCGAGGACACGGCCGCATATTTCTGTGTGATAGATCTTGACTATTGGGGTCAGGGAAC
CCTGGTCACCGTCTCCTCAGCCTCCACCAAGGGCCCATCGGTCTTCCCCCTGGCGCCCTGCTCCAGGAGCACCTCCGAGA
GCACAGCCGCCCTGGGCTGCCTGGTCAAGGACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCCCTCAGCAGCGTGGTGACCGTGCCCTCCAG
CAGCTTGGGCACGAAGACCTACACCTGCAACGTAGATCACAAGCCCAGCAACACCAAGGTGGACAAGAGAGTTGAGTCCA
AATATGGTCCCCCATGCCCACCCTGCCCAGCACCTGAGTTCCTGGGGGGACCATCAGTCTTCCTGTTCCCCCCAAAACCC
AAGGACACTCTCATGATCTCCCGGACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGAGGAGCAGTTCAACAGCACGTACC
GTGTGGTCAGCGTCCTCACCGTCCTGCACCAGGACTGGCTGAACGGCAAGGAGTACAAGTGCAAGGTCTCCAACAAAGGC
CTCCCGTCCTCCATCGAGAAAACCATCTCCAAAGCCAAAGGGCAGCCCCGAGAGCCACAGGTGTACACCCTGCCCCCATC
CCAGGAGGAGATGACCAAGAACCAGGTCAGCCTGACCTGCCTGGTCAAAGGCTTCTACCCCAGCGACATCGCCGTGGAGT
GGGAGAGCAATGGGCAGCCGGAGAACAACTACAAGACCACGCCTCCCGTGCTGGACTCCGACGGCTCCTTCTTCCTCTAC
AGCAGGCTCACCGTGGACAAGAGCAGGTGGCAGGAGGGGAATGTCTTCTCATGCTCCGTGATGCATGAGGCTCTGCACAA
CCACTACACACAGAAGTCCCTCTCCCTGTCTCTGGGTAAATGA
Heavy chain polypeptide
(SEQ ID NO: 196)
EVQLVESGGGLVQPGGSLRLSCAASGFTFSDYAMSWVRQAPGKGLEWVSGISGNGGDTYYGDFVKGRFTISRDNSKNTLY
LQMNSLRGEDTAAYFCVIDLDYWGQGTLVTVSSASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPPVLDSDGSFFLY
SRLTVDKSRWQEGNVFSCSVMHEALHNHYTQKSLSLSLGK
Light chain DNA
(SEQ ID NO: 197)
GACATCCAGATGACCCAGTCTCCTTCCACCCTGTCTGCATCTGAAGGAGACAGAGTCACCATCACTTGCCGGGCCAGTCA
GAGTATTAGTAGCTGGTTGGCCTGGTATCAACAGAAACCAGGAAAAGCCCCTAGGCTCCTGATCTATAAGGCGTCTATTT
TAGGAGATGGGGTCCCATCAAGGTTCAGCGGCAGTGGATCTGGGACAGAATTCACTCTCACCATCAGCAGCCTGCAGCCT
GATGATTTTGCTACTTATTACTGCCACCAGTATAATAGTTATTTGTGGACGTTCGGCCAAGGGACCAAGGTGGAAATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 198)
DIQMTQSPSTLSASEGDRVTITCRASQSISSWLAWYQQKPGKAPRLLIYKASILGDGVPSRFSGSGSGTEFTLTISSLQP
DDFATYYCHQYNSYLWTFGQGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H14728P
Heavy chain DNA
(SEQ ID NO: 199)
CAGGTGCAGCTGCAGGAGTCGGGCCCAGGACTGGTGAAGCCTTCACAGACCCTGTCCCTCACCTGCACTGTCTCTGGTGG
CTCCATCAGCAGTGCTGATTACTATTGGAGCTGGATCCGCCAGCACCCAGGGAAGGGCCTGGAGTGGATTGGATCCATCT
ATTATACTGGGAGTACTTACTACAACCCGTCCCTCAAGAGTCGACTTACCATATCAATAGACACGTCTGAGAACCAGTTC
TCTTTGAAACTGACCTCTCTGACTGCCGCGGACACGGCCGTGTATTACTGTGCGAGCGAGGAGGCTAACTGGGGATCCCA
CTTTGACTCCTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAATGA
Heavy chain polypeptide
(SEQ ID NO: 200)
QVQLQESGPGLVKPSQTLSLTCTVSGGSISSADYYWSWIRQHPGKGLEWIGSIYYTGSTYYNPSLKSRLTISIDTSENQF
SLKLTSLTAADTAVYYCASEAANWGSHFDSWGQGTLVTVSSASTKGPSVFPLAPCSRSTSESTAALGCLVKDYFPEPVTV
WSNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
Light chain DNA
(SEQ ID NO: 201)
GACATCCAGATGACCCAGTCTCCATCCTCCCTGTCTGCATCTGTAGGAGACAGAGTCACCATCACTTGCCGGGCAAGTCA
GAGCATTGACAACTTTTTAAATTGGTATCAGCAGAAACCAGGGAAAGCCCCTAAGCTCCTGATCTATGCTGCATCCAGTT
TGCAAAGTGGGGTCCCATCAAGGTTCAGTGGCAGTGGATCTGGGACAGATTTCACTCTCACCATCAGCAGTCTGCAACCT
GAAGATTTTGCATCTTACTACTGTCAACATAGTCACAGTGCCCATCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGT
Light chain polypeptide
(SEQ ID NO: 202)
DIQMTQSPSSLSASVGDRVTITCRASQSIDNFLNWYQQKPGKAPKLLIYAASSLQSGVPSRFSGSGSGTDFTLTISSLQP
EDFACYYCQHSHSAHPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
H4H14731P
Heavy chain DNA
(SEQ ID NO: 203)
CAGCTGCAGCTGCAGGAGTCGGGCCCAGGACTGGTGAAGCCTTCGGAGACCCTGTCCCTCACCTGCACTGTCTCTGGTGG
CTCCATCAGCAGTAGTAATTACTACTGGGGCTGGATCCGCCAGCCCCCAGGGAAGAGACTGGAGTGGATTGGGAGTATCT
ATTATAGTGGGAGCACCTACTACAACCCGTCCCTCAAGACTCGAGTCACCATATCCGTAGACACGTCCAAGAATCAGTTC
TCCCTGAAGCTGACCTCTGTGACCGCCGCAGACACGGCTGTGTATTACTGTGCGAGAGAGGAAGCAGCAGCTTTGACGCA
CTTTGACTTCTGGGGCCAGGGAACCCTGGTCACCGTCTCCTCAGCCTCCACCAAGGGCCCATCGGTCTTCCCCCTGGCGC
CCTGCTCCAGGAGCACCTCCGAGAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGTTCCTGGGGGGACCATCA
GTCTTCCTGTTCCCCCCAAAACCCAAGGACACTCTCATGATCTCCCGGACCCCTGAGGTCACGTGCGTGGTGGTGGACGT
GAGCCAGGAAGACCCCGAGGTCCAGTTCAACTGGTACGTGGATGGCGTGGAGGTGCATAATGCCAAGACAAAGCCGCGGG
AGGAGCAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACCACGCCTCCCGTGCTGGAC
TCCGACGGCTCCTTCTTCCTCTACAGCAGGCTCACCGTGGACAAGAGCAGGTGGCAGGAGGGGAATGTCTTCTCATGCTC
CGTGATGCATGAGGCTCTGCACAACCACTACACACAGAAGTCCCTCTCCCTGTCTCTGGGTAAATGA
Heavy chain polypeptide
(SEQ ID NO: 204)
QLQLQESGPGLVKPSETLSLTCTVSGGSISSSNYYWGWIRQPPGKRLEWIGSIYYSGSTYYNPSLKTRVTISVDTSKNQF
SLKLTSVTAADTAVYYCAREEAAALTHFDFWGQGTLVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
Light chain DNA
(SEQ ID NO: 205)
GACATCCAGATGACCCAGTCTCCATCCTCCCTGTCTGCATCTGTAGGAGACAGAGTCACCATCACTTGCCGGGCAAGTCA
GAGCATTAGCAACTATTTAAATTGGTATCAGCAGAAACCAGGGAAAGCCCCTAAGCTCCTGATCTTTGCTGCATCCAGTT
TACAAAGTGGGGTCCCATCAAGGTTCAGTGGCAGTGGATCTGGGACAGATTTCACTCTCACCATCAGCAGTCTGCAACCT
GAAGATTTTGCAACTTACTACTGTCAACATAGTCACAGTTCCCATCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGT
Light chain polypeptide
(SEQ ID NO: 206)
DIQMTQSPSSLSASVGDRVTITCRASQSISNYLNWYQQKPGKAPKLLIFAASSLQSGVPSRFSGSGSGTDFTLTISSLQP
EDFATYYCQHSHSSHPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
H4H14732P
Heavy chain DNA
(SEQ ID NO: 207)
GAAGTGCAGCTGGTGGAGTCTGGGGGAGGCTTGGTACAGCCTGGCAGGTCCCTGAGACTCTCCTGTGCAGCCTCTGGATT
CACCTTTGATGATTATGCCATGCACTGGGTCCGGCAAGCTCCAGGGAAGGGCCTGGAGTGGGTCTCAGGTATTAATTGGG
CTGGTTATAACATAGACTATGCGGACTCTGTGAAGGGCCGATTCACCATCTCCAGAGACAACGCCAAGAACTCCCTGTAT
CTGCAAATGAACAGTCTGAGAGCTGAGGACACGGCCTTGTATTACTGTGCAAAAGATATGCGTGGATTCAGTTATGGTTT
CCCCTTTGACTACTGGGGCCAGGGAACCCTGGTCACCGTCTCCTCAGCCTCCACCAAGGGCCCATCGGTCTTCCCCCTGG
CGCCCTGCTCCAGGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGCACCTGAGTTCCTGGGGGGACCA
TCAGTCTTCCTGTTCCCCCCAAAACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTCTGGGTAAATGA
Heavy chain polypeptide
(SEQ ID NO: 208)
EVQLVESGGGLVQPGRSLRLSCAASGFTFDDYAMHWVRQAPGKGLEWVSGINWAGYNIDYADSVKGRFTISRDNAKNSLY
LQMNSLRAEDTALYYCAKDMRGFSYGFPFDYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK
Light chain DNA
(SEQ ID NO: 209)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light chain polypeptide
(SEQ ID NO: 210)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKTVAAPSVFIFPPSDEQLKSGTASVVCLLNNFYPREAKVQWKVDNAKQSGNSQ
ESVTEQDSKDSTYSLSSTLTLSKADYEKHKVYACEVTHQGLSSPVTKSFNRGEC
H4H14734P2
Heavy chain DNA
(SEQ ID NO: 211)
GAGGTGCAGCTGGTGGAGTCTGGGGGAGGCTTGGTAAAGCCGGGGGGGTCCCTTAGACTCTCCTGTGCAGCCTCTGGATT
TATTTTCAGTAACGCCTGGATGAACTGGGTCCGCCAGGCTCCAGGGAAGGGACTGGCGTGGGTTGGCCGTATTAAAACCG
AAACTGATGGTGGGACAACAGACTACGCTGCACCCGTAAAAGGCAGATTCACCATCTCAAGAGATGACTCAAAAAACACG
CTGTATCTGCAAATGAACAGCGTGAAAACCGAGGACACAGCCGTGTATTACTGTACAGGGGGATACAGCTATGGTGACGA
TAGCAGCAGCTGGAACGAGGGCTACTACTACTACGGTATGGACGTCTGGGGCCAAGGGACCACGGTCACCGTCTCCTCAG
CCTCCACCAAGGGCCCATCGGTCTTCCCCCTGGCGCCCTGCTCCAGGAGCACCTCCGAGAGCACAGCCGCCCTGGGCTGC
CTGGTCAAGGACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGCACGAAGACCT
ACACCTGCAACGTAGATCACAAGCCCAGCAACACCAAGGTGGACAAGAGAGTTGAGTCCAAATATGGTCCCCCATGCCCA
CCCTGCCCAGCACCTGAGTTCCTGGGGGGACCATCAGTCTTCCTGTTCCCCCCAAAACCCAAGGACACTCTCATGATCTC
CCGGACCCCTGAGGTCACGTGCGTGGTGGTGGACGTGAGCCAGGAAGACCCCGAGGTCCAGTTCAACTGGTACGTGGATG
GCGTGGAGGTGCATAATGCCAAGACAAAGCCGCGGGAGGAGCAGTTCAACAGCACGTACCGTGTGGTCAGCGTCCTCACC
GTCCTGCACCAGGACTGGCTGAACGGCAAGGAGTACAAGTGCAAGGTCTCCAACAAAGGCCTCCCGTCCTCCATCGAGAA
AACCATCTCCAAAGCCAAAGGGCAGCCCCGAGAGCCACAGGTGTACACCCTGCCCCCATCCCAGGAGGAGATGACCAAGA
ACCAGGTCAGCCTGACCTGCCTGGTCAAAGGCTTCTACCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTACAGCAGGCTCACCGTGGACAA
GAGCAGGTGGCAGGAGGGGAATGTCTTCTCATGCTCCGTGATGCATGAGGCTCTGCACAACCACTACACACAGAAGTCCC
TCTCCCTGTCTCTGGGTAAATGA
Heavy chain polypeptide
(SEQ ID NO: 212)
EVQLVESGGGLVKPGGSLRLSCAASGFIFSNAWMNWVRQAPGKGLAWVGRIKTETDGGTTDYAAPVKGRFTISRDDSKNT
LYLQMNSVKTEDTAVYYCTGGYSYGDDSSSWNEGYYYYGMDVWGQGTTVTVSSASTKGPSVFPLAPCSRSTSESTAALGC
LVKDYFPEPVTVSWNSGALTSGVHTFPAVLQSSGLYSLSSVVTVPSSSLGTKTYTCNVDHKPSNTKVDKRVESKYGPPCP
PCPAPEFLGGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSLSLSLGK
Light chain DNA
(SEQ ID NO: 213)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light chain polypeptide
(SEQ ID NO: 214)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H14757P
Heavy chain DNA
(SEQ ID NO: 215)
GAAGTGCAGCTGGTGGAGTCTGGGGGAGGCTTGGTACAGCCTGGCAGGTCCCTGAGACTCTCCTGTACAGCCTCTGGATT
CACCTTTGATGATTATGCCATGCACTGGGTCCGGCAAGCTCCAGGGAAGGGCCTGGAGTGGGTCTCAGGTATTCGTTGGA
ATGGTGGTAGTATAGGCTATGTGGACTCTGTGAAGGGCCGATTCACCATCTCCAGAGACAACGCCAAGAAGTCCCTGCAT
CTGCAAATGAACAGTCTAAAAACTGAGGACACGGCCTTGTATTACTGTGCAAAAGATATAGGCGATATTTTGACTGGTTT
TTATGGAGAATACGGAATGGACGTCTGGGGCCAAGGGACCACGGTCACCGTCTCCTCAGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA
Heavy chain polypeptide
(SEQ ID NO: 216)
EVQLVESGGGLVQPGRSLRLSCTASGFTFDDYAMHWVRQAPGKGLEWVSGIRWNGGSIGYVDSVKGRFTISRDNAKKSLH
LQMNSLKTEDTALYYCAKDIGDILTGFYGEYGMDVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK
Light chain DNA
(SEQ ID NO: 217)
GACATCCAGATGACCCAGTCTCCATCCTCCCTGTCTGCATCTGAAGGAGACAGAGTCACCATCACTTGCCGGGCAAGTCA
GAGCATTAGCAGCTATTTAAATTGGTATCAGCAGAAAGCAGGGAAAGCCCCTAACCTCCTGATCTATGCTGCATCCAGTT
TGCAAAGTGGGGTCCCATCAAGGTTCAGTGGCAGTGGATCTGGGACAGAGTACACTCTCACCATCAGCAGTCTGCAACCT
GAAGATTTTGCAACTTACTACTGTCAACAGAGTTACATTATCCCGTACACTTTTGGCCAGGGGACCAAGCTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 218)
DIQMTQSPSSLSASEGDRVTITCRASQSISSYLNWYQQKAGKAPNLLIYAASSLQSGVPSRFSGSGSGTEYTLTISSLQP
EDFATYYCQQSYIIPYTFGQGTKLEIKRTVAAPSVFIFPPSDEQLKSGTASVVCLLNNFYPREAKVQWKVDNALQSGNSQ
ESVTEQDSKDSTYSLSSTLTLSKADYEKHKVYAVEVTHQGLSSPVTKSFNRGEC
H4H14758P
Heavy chain DNA
(SEQ ID NO: 219)
GAAGTGCAGCTGGTGGAGTCTGGGGGAGGGTTGGTACAGCCTGGCAGGTCCCTGAGACTCTCCTGTGCAGCCTCTGGATT
CACCTTTGATGATTATGCCATGCACTGGGTCCGGCAAGCTCCAGGGAAGGGCCTGGAGTGGGTCTCAAGTGTTAGGTGGA
ATGGTGGTATTATAGGCTATGCGGACTCTGTGAAGGGCCGATTCACCATCTCCAGAGACAACGCCAAGAACTCCCTGTAT
CTGCAAATGAACAGTCTGAGACCTGAGGACACGGCCCTCTATTACTGTGCAAAAGATATAGGCGATGTTTTGACTGGTTA
TTATGGAGAATACGGTATGGACGTCTGGGGCCAAGGGACCACGGTCACCGTCTCCTCAGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA
Heavy chain polypeptide
(SEQ ID NO: 220)
EVQLVESGGGLVQPGRSLRLSCAASGFTFDDYAMHWVRQAPGKGLEWVSSVRWNGGIIGYADSVKGRFTISRDNAKNSLY
LQMNSLRPEDTALYYCAKDIGDVLTGYYGEYGMDVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESGYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK
Light chain DNA
(SEQ ID NO: 221)
GACATCCAGATGACCCAGTCTCCATCCTCCCTGTCTGCATCTGTGGGAGACAGAGTCACCATCGCTTGCCGGGCAAGTCA
GAGCATTACCACCTATTTAAATTGGTATCAGCAGAAACCAGGGAAAGCCCCTAAACTCCTGATCTATGCTGCATCCAGTT
TGCAAAGTGGGGTCCCATCAAGGTTCAGTGGCAGTGGATCTGGGACAGATTTCACTCTCACCATCAGTAGTCTGCAACCT
GAAGATTTTGCAACTTACTACTGTCAACAGAGTTACATTTCCCCGTACACTTTTGGCCAGGGGACCAAGCTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GT
Light chain polypeptide
(SEQ ID NO: 222)
DIQMTQSPSSLASAVGDRVTIACRASQSITTYLNWYQQKPGKAPKLLIYAASSLQSGVPSRFSGSGSGTDFTLTISSLQP
EDFATYYCQQSYISPYTFGQGTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRVEC
H4H14760P2
Heavy chain DNA
(SEQ ID NO: 223)
CAGGTGCAGCTGGTGGAGTCTGGGGGAGGCGTGGTCCAGCCTGGGAAGTCCCTGAGACTCTCCTGTGCAGCCTCTGGATT
CACCTTCAGTAATTATGGCATACACTGGGTCCGCCAGGCTCCAGGCAAGGGGCTGGAGTGGGTGGCGATTATATTATATG
ATGGAAGTAATCAACACTATGCAGATTCCGTGAAGGGCCGATTCACCATTTCCAGAGACAATTCCAAAAACACGCTGTAT
CTTCAAATGAACAACCTGAGAGCTGAGGACACGGCCGTTTATTACTGTGCGAGAGATCTTGATCTTTGGAGTGGTTATTA
TACAAACGGGGACGGTATGGACGTCTGGGGCCAAGGGACCACGGTCACCGTCTCCTCAGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA
Heavy chain polypeptide
(SEQ ID NO: 224)
QVQLVESGGGVVQPGKSLRLSCAASGFTFSNYGIWHVRQAPGKGLEWVAIILYDSSNQHYADSVKGRFTISRDNSKNTLY
LQMNNLRAEDTAVYYCARDLDLWSGYYTNGDGMDVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLDKGFYPSDIAVEWESNGQPENNYKTT
PPVLDSDGSFFLYSRLTVDKSRWQEGNVFSCSVMHEALHNHYTQKSLSLSLGK
Light chain DNA
(SEQ ID NO: 225)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light chain polypeptide
(SEQ ID NO: 226)
DIQMTQSPSSLSASVGDRVTITCRASQSISSYLNWYQQKPGKAPKLLIYAASSLQSGVPSRFSGSGSGTDFTLTISSLQP
EDFATYYCQQYSYTPPITFGQGTRLEIKTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC

### Example 2: Bioassay with HEK293/D9 (NFκB-Luciferase)/hIL-36R and HEK293/NFκB-Luciferase/mfIL-36R Cells

IL-36 receptor (IL-36R) is a single-pass membrane receptor for a subset of members of the IL-1 family of cytokines, IL-36α, IL-36β, and IL-367, and upon binding to these ligands, there is recruitment of its co-receptor, the IL-1R accessory protein (IL-1RAcP), which induces a signaling cascade that involves NFκB and mitogen-activated kinase pathways (Sims et al, 2010). A bioassay was developed to detect the transcriptional activation by NFκB via IL-36R activation using reporter cell lines that stably express either full-length human IL-36R (hIL-36R; amino acids 1 through 575 of accession number NP_003845.2) or Macaca fascicularis IL-36R (MfIL-36R) along with a luciferase reporter [NFκB response element (5×)-luciferase-IRES-GFP] in HEK293 cells. IL-1RAcP is endogenously expressed in the HEK293 cell line. The resulting stable cell lines, referred to as HEK293/NFκB-luc/hIL-36R and HEK293/NFκB-luc/MfIL-36R, was isolated and maintained in DMEM containing 10% FBS, NEAA, penicillin/streptomycin/glutamine, and 500 μg/mL G418.

For the bioassay, cells were seeded into 96-well assay plates at 10,000 cells/well in OPTIMEM supplemented with 0.1% FBS and then incubated at 37° C. in 5% CO₂ overnight. The next day, to determine the dose response of ligands, human IL-36a (hIL-36α; R&D Systems, #6995/IL), human IL-36β (hIL-36β; R&D Systems, #6334-IL), or human IL-36γ (hIL-367; R&D Systems, #6835-IL) were serially diluted at 1:3 (from 10 nM to 0.0002 nM) and added to cells. A control containing dilution buffer but no IL-36 ligand was also added to one sample of cells. To measure inhibition, antibodies were serially diluted at 1:3 (from 100 nM to 0.002 nM) plus a control sample containing no antibody and pre-incubated with the cells followed by addition of constant concentrations of hIL-36α, hIL-36β, or hIL-367. For testing with HEK293/NFκB-luc/hIL-36R cells, 20 pM of hIL-36α, 15 pM of hIL-36β, or 10 pM of hIL-36γ was used as a constant concentration and for testing with HEK293/NFκB-luc/mfIL-36R cells, 500 pM of hIL-36α, 600 pM of hIL-36β, or 300 pM of hIL-36γ was used as a constant concentration. After 5.5 hours of incubation at 37° C. in 5% CO₂, OneGlo reagent (Promega, #E6051) was added to the samples and luciferase activity was then measured using a Victor X (Perkin Elmer) plate reader.

The results were analyzed using nonlinear regression (4-parameter logistics) with Prism 6 software (GraphPad) to obtain EC₅₀ and IC₅₀ values. To determine the maximum inhibition, the range between the maximum and minimum RLU values for each antibody was calculated as a percentage of the RLU range between no IL-36 ligand and the constant amount of IL-36 ligand used per assay.

As shown in Table 2-1, 9 out of 12 anti-IL-36R antibodies of the invention tested completely blocked the stimulation of HEK293/NFkB-luc/hIL-36R cells by 20 pM hIL-36a with IC₅₀ values ranging from 100 pM to 970 pM. One of the IL-36R antibodies tested demonstrated partial blockade of hIL-36a stimulation of HEK293/NFkB-luc/hIL-36R cells with a maximum percent blockade of 22%. One of the IL-36R antibodies tested demonstrated weak blockade of hIL-36a stimulation of HEK293/NFkB-luc/hIL-36R cells with a maximum percent blockade of 61%, while another of the anti-IL-36R antibodies tested did not demonstrated any inhibition of hIL-36a stimulation. Six out of 12 anti-IL-36R antibodies of the invention tested completely blocked the stimulation of HEK293/NFkB-luc/hIL-36R cells by 15 pM hIL-36β with IC₅₀ values ranging from 120 pM to 1.3 nM. One of the IL-36R antibodies tested demonstrated weak blockade of hIL-360 stimulation of HEK293/NFkB-luc/hIL-36R cells with a maximum percent blockade of 69% and 5 anti-IL-36R antibodies tested did not demonstrate measurable inhibition of hIL-36β stimulation. Six out of 12 anti-IL-36R antibodies of the invention tested completely blocked the stimulation of HEK293/NFkB-luc/hIL-36R cells by 10 pM hIL-36γ with IC₅₀ values ranging from 120 pM to 1.2 nM. Four anti-IL-36R antibodies of the invention tested demonstrated partial blockade of hIL-36γ stimulation with maximum percent blockade ranging from 24 to 87%. One anti-IL-36R antibody of the invention tested showed weak blockade of hIL-36γ stimulation with maximum percent blockade of 69%, and one anti-IL36R antibody of the invention did not demonstrate inhibition of hIL-36γ stimulation. The isotype control antibody tested did not demonstrate inhibition of IL-36 ligand stimulation of the HEK293/NFkB-luc/hIL-36R cells. As shown in Table 2-1, hIL-36α, hIL-36β, and hIL-36γ activated HEK293/NFkB-luc/hIL-36R cells with EC₅₀ values of 12 pM, 14 pM, and 8.4 pM respectively.

As shown in Table 2-2, six out of 12 anti-IL-36R antibodies of the invention tested completely or nearly completely blocked the stimulation of HEK293/NFkB-luc/MfIL-36R cells by 500 pM hIL-36a with IC₅₀ values ranging from 60 pM to 3.1 nM. Two anti-IL-36R antibodies of the invention tested demonstrated weak blockade of hIL-36a stimulation of HEK293/NFkB-luc/MfIL-36R cells with maximum percent blockade of 29 and 47%, while 4 anti-IL-36R antibodies did not show inhibition of hIL-36a stimulation of this cell line. Six out of 12 anti-IL-36R antibodies of the invention tested completely or nearly completely blocked the stimulation of HEK293/NFkB-luc/MfIL-36R cells by 600 pM hIL-36β with IC₅₀ values ranging from 120 pM to 7.1 nM. Three anti-IL-36R antibodies of the invention tested demonstrated weak blockade of hIL-36β stimulation of HEK293/NFkB-luc/MfIL-36R cells with maximum percent blockade ranging from 36 to 48%, while three anti-IL-36R antibodies of the invention did not show inhibition of hIL-36β stimulation of this cell line. Six out of anti-IL-36R antibodies of the invention tested completely or nearly completely blocked the stimulation of HEK293/NFkB-luc/MfIL-36R cells by 300 pM hIL-36γ with IC₅₀ values ranging from 85 pM to 5.4 nM. Three anti-IL-36R antibodies of the invention tested showed weak blockade of hIL-36γ stimulation of HEK293/NFkB-luc/MfIL-36R cells with maximum percent blockade ranging from 25 to 43%, while three anti-IL-36R antibodies of the invention did not show inhibition of hIL-36γ stimulation of this cell line. The isotype control antibody tested did not demonstrate inhibition of IL-36 ligand stimulation of the HEK293/NFkB-luc/MfIL-36R cells. As shown in Table 2-1, hIL-36α, hIL-36β, and hIL-36γ activated HEK293/NFkB-luc/MfIL-36R cells with EC₅₀ values of 170 pM, 270 pM, and 62 pM respectively.

TABLE 2-1
Anti-IL-36R antibody inhibition of stimulation of HEK293/
NFκB-luc/hIL-36R cells by hIL-36 ligands.

| Ligand | hIL-36α | | hIL-36β | | hIL-36γ | |
|---|---|---|---|---|---|---|
| EC₅₀ | 1.2E−11M | | 1.4E−11M | | 8.4E−12M | |
| Constant | 20pM | | 15pM | | 10pM | |
| | | Max | | Max | | Max |
| | | Inhibition | | Inhibition | | Inhibition |
| Antibodies | IC₅₀ [M] | (%) | IC₅₀ [M] | (%) | IC₅₀ [M] | (%) |
| H4H14699P2 | 1.3E−10 | 100 | 1.3E−10 | 100 | 1.4E−10 | 99 |
| H4H14700P2 | 1.9E−10 | 101 | 2.0E−10 | 100 | 12E−10 | 100 |
| H4H14706P2 | 1.0E−10 | 101 | 1.2E−10 | 101 | 1.2E−10 | 99 |
| H4H14708P2 | 1.3E−10 | 101 | 2.0E−10 | 100 | 1.6E−10 | 99 |
| H4H14709P | 1.4E−10 | 22 | No | No | 1.3E−10 | 24 |
| | (partial) | | inhibition | inhibition | (partial) | |
| H4H14728P | 9.7E−10 | 97 | 1.3E−09 | 99 | 1.2E−09 | 99 |
| H4H14731P | 7.8E−10 | 99 | 9.4E−10 | 99 | 7.3E−10 | 99 |
| H4H14732P2 | >1.0E−08 | 61 | >1.0E−08 | 69 | >1.0E−08 | 69 |
| H4H14734P2 | No | No | No | No | No | No |
| | inhibition | inhibition | inhibition | inhibition | inhibition | inhibition |
| H4H14757P | 1.8E−10 | 101 | No | No | 1.3E-10 | 87 |
| | | | inhibition | inhibition | (partial) | |
| H4H14758P | 1.2E−10 | 100 | No | No | 1.6E−10 | 57 |
| | | | inhibition | inhibition | (partial) | |
| H4H14760P2 | 4.9E−10 | 99 | No | No | 7.0E−10 | 49 |
| | | | inhibition | inhibition | (partial) | |
| Isotype | No | No | No | No | No | No |
| control | inhibition | inhibition | inhibition | inhibition | inhibition | inhibition |
| antibody | | | | | | |

TABLE 2-2
Anti-IL-36R antibody inhibition of stimulation of HEK293/
NFκB-luc/MfIL-36R cells by hIL-36 ligands.

| Ligand | hIL-36α | | hIL-36β | | hIL-36γ | |
|---|---|---|---|---|---|---|
| EC₅₀ | 1.7E-10M | | 2.7E-10M | | 6.2E-11M | |
| Constant | 500pM | | 600pM | | 300pM | |
| | | Max | | Max | | Max |
| | | Inhibition | | Inhibition | | Inhibition |
| Antibodies | IC₅₀ [M] | (%) | IC₅₀ [M] | (%) | IC₅₀ [M] | (%) |
| H4H14699P2 | 8.4E−11 | 97 | 1.9E−10 | 98 | 2.0E−10 | 97 |
| H4H14700P2 | 1.2E−10 | 99 | 1.8E−10 | 99 | 1.8E−10 | 99 |
| H4H14706P2 | 6.0E−11 | 100 | 1.2E−10 | 100 | 8.5E−11 | 100 |
| H4H14708P2 | 8.9E−11 | 99 | 1.2E−10 | 100 | 1.2E−10 | 100 |
| H4H14709P | No | No | No | No | No | No |
| | inhibition | inhibition | inhibition | inhibition | inhibition | inhibition |
| H4H14728P | 1.3E−09 | 93 | 1.5E−09 | 95 | 2.0E−09 | 93 |
| H4H14731P | 3.1E−09 | 84 | 7.1E−09 | 78 | 5.4E−09 | 75 |
| H4H14732P2 | >1.0E−07 | 47 | >1.0E−07 | 43 | >1.0E−07 | 36 |
| H4H14734P2 | No | No | No | No | No | No |
| | inhibition | inhibition | inhibition | inhibition | inhibition | inhibition |
| H4H14757P | >1.0E−07 | 29 | >1.0E−07 | 36 | >1.0E−07 | 25 |
| H4H14758P | No | No | No | No | No | No |
| | inhibition | inhibition | inhibition | inhibition | inhibition | inhibition |
| H4H14760P2 | No | No | >1.0E−07 | 48 | >1.0E−07 | 43 |
| | inhibition | inhibition | | | | |
| Isotype | No | No | No | No | No | No |
| control | inhibition | inhibition | inhibition | inhibition | inhibition | inhibition |
| antibody | | | | | | |

### Example 3: IL-36R Octet Cross-Competition

Binding competition between a panel of different anti-IL-36R antibodies was determined using a real time, label-free bio-layer interferometry assay on an Octet® HTX biosensor (ForteBio, A Division of Pall Life Sciences). The entire experiment was performed at 25° C. in 0.01 M HEPES pH 7.4, 0.15 M NaCl, 3 mM EDTA, 0.05% v/v Surfactant Tween-20, 0.002% NaN₃ and 1 mg/mL BSA (HBS-ET kinetics buffer) with the plate shaking at the speed of 1000 rpm. To assess whether two antibodies are able to compete with one another for binding to their respective epitopes on the recombinant human IL-36R extracellular domain expressed with a C-terminal myc-myc-hexahistidine tag (hIL-36R-MMH: mROR1 signal sequence (M1-A29)-human IL36R (D20-Y337)-mycmycHis₆), around 0.3 nM of hIL-36R-MMH was first captured onto anti-His antibody coated Octet biosensors (Fortebio Inc, #18-5079) by submerging the biosensors for 3 minutes into wells containing 30 μg/mL of hIL-36R-MMH. The antigen-captured biosensors were then saturated with a first anti-IL-36R antibody (subsequently referred to as mAb-1) by submerging into wells containing 50 μg/mL solution of mAb-1 for 4 minutes. The biosensors were then subsequently submerged into wells containing a 50 μg/mL solution of a second anti-IL-36R antibody (subsequently referred to as mAb-2) for 3 minutes. The biosensors were washed in HBS-ET kinetics buffer in between every step of the experiment. The real-time binding response was monitored during the entire course of the experiment and the maximum binding response for all the steps was recorded. The response of mAb-2 binding to hIL-36R-MMH pre-complexed with mAb-1 was compared and competitive/non-competitive behavior of different anti-IL-36R antibodies was determined as shown in Table 3-1.

TABLE 3-1
Cross-competition of anti-IL-36R antibodies for binding
to human IL-36R-MMH

| First antibody | Second antibody |
|---|---|
| (mAb-1) binding | (mAb-2) shown to |
| to captured hIL- | compete with |
| 36R-MMH | mAb-1 |
| H4H14699P2 | H4H14700P2 |
| | H4H14706P2 |
| | H4H14708P2 |
| | H4H14732P2 |
| H4H14700P2 | H4H14699P2 |
| | H4H14706P2 |
| | H4H14708P2 |
| | H4H14732P2 |
| H4H14706P2 | H4H14699P2 |
| | H4H14700P2 |
| | H4H14708P2 |
| | H4H14732P2 |
| H4H14708P2 | H4H14699P2 |
| | H4H14700P2 |
| | H4H14706P2 |
| | H4H14732P2 |
| H4H14732P2 | H4H14699P2 |
| | H4H14700P2 |
| | H4H14706P2 |
| | H4H14708P2 |
| H4H14757P | H4H14758P |
| | H4H14760P2 |
| H4H14758P | H4H14757P |
| | H4H14760P2 |
| H4H14760P2 | H4H14757P |
| | H4H14758P |
| H4H14728P | H4H14731P |
| H4H14731P | H4H14728P |
| H4H14709P | H4H14734P2 |
| H4H14734P2 | H4H14709P |

### Example 4: Antibody Binding Kinetics

Equilibrium dissociation constants (K_{D} values) for IL-36R binding to purified anti-IL-36R antibodies were determined using a real-time surface plasmon resonance biosensor using a Biacore 4000 instrument. The Biacore sensor surface was first derivatized by amine coupling with a monoclonal mouse anti-human Fc antibody (GE, #BR-1008-39) to capture anti-IL-36R monoclonal antibodies. All binding studies were performed in 0.01 M Hepes pH 7.4, 0.15 M NaCl, 3 mM EDTA, and 0.05% v/v Surfactant Tween-20 (HBS-ET running buffer) at 25° C. and 37° C. Different concentrations of IL-36R reagents, human IL-36R extracellular domain expressed with a C-terminal myc-myc-hexahistidine tag (hIL-36R-MMH), Macaca fascicularis IL-36R extracellular domain expressed with a C-terminal myc-myc-hexahistidine tag (mfIL-36R-MMH: mROR1 signal sequence (M1-A29). Macaca fascicularis IL36R_ecto domain (D20-A336).mycmycHis6), human IL-36R extracellular domain expressed with a C-terminal mouse IgG2a Fc tag (hIL-36R-mFc: mROR1 signal sequence (M1-A29)-human IL36R (D20-Y337)-mouse IgG2aFc (E98-K330)) or an in-line fusion protein of human IL-36R extracellular domain and IL1RAcP extracellular domain expressed with mouse IgG2a Fc tag (hIL-36R-Trap-mFc: mROR1 signal sequence (M1-A29)-human IL36R ecto domain (D20-Y337)-human IL1RacP ecto domain (S21-E359)-mouse IgG2aFc) in HBS-ET running buffer (ranging from 100 nM to 3.7 nM, 3-fold dilutions) were injected over the anti-IL-36R antibody captured surface for 4 minutes at a flow rate of 30 μL/minute and their dissociation in HBS-ET running buffer was monitored for 10 minutes. Kinetic association rate constant (kₐ) and dissociation rate constant (k_{d}) were determined by fitting the real-time sensorgrams to a 1:1 binding model using Scrubber 2.0c curve fitting software. Binding dissociation equilibrium constants (K_{D}) and dissociative half-lives (t½) were calculated from the kinetic rate constants as:

$${K}_{D} ( M ) = \frac{kd}{ka} , and t \frac{1}{2} ( min ) = \frac{ln ( 2 )}{60 * kd}$$

Binding kinetic parameters for hIL-36R-MMH, mfIL-36R-MMH or hIL-36R.mFc binding to different anti-IL-36R antibodies of the invention at 25° C. and 37° C. are shown in Tables 4-1 through 4-8. At 25° C., hIL-36R-MMH bound to all of the anti-IL-36R antibodies of the invention with K_{D} values ranging from 2.18 nM to 13.9 nM, as shown in Table 4-1. At 37° C., hIL-36R-MMH bound to all of the anti-IL-36R antibodies of the invention with K_{D} values ranging from 4.25 nM to 29.5 nM, as shown in Table 4-2. At 25° C., mfIL-36R-MMH bound to 9 of the 12 anti-IL-36R antibodies of the invention with K_{D} values ranging from 7.87 nM to 34.4 nM, as shown in Table 4-3. At 37° C., mfIL-36R-MMH bound to 9 of the 12 anti-IL-36R antibodies of the invention with K_{D} values ranging from 14.4 nM to 58.2 nM, as shown in Table 4-4. At 25° C., hIL-36R-mFc bound to 11 of the 12 anti-IL-36R antibodies of the invention with K_{D} values ranging from 173 pM to 5.79 nM, as shown in Table 4-5. One anti-IL-36R antibody of the invention demonstrated inconclusive binding to hIL-36R-mFc under the experimental conditions at 25° C. At 37° C., hIL-36R-mFc bound to all of the anti-IL-36R antibodies of the invention with K_{D} values ranging from 205 pM to 28.7 nM, as shown in Table 4-6. At 25° C., hIL-36R-Trap-mFc bound to all of the anti-IL-36R antibodies of the invention with K_{D} values ranging from 212 pM to 14 nM, as shown in Table 4-7. At 37° C., hIL-36R-Trap-mFc bound to all of the anti-IL-36R antibodies of the invention with K_{D} values ranging from 264 pM to 40.9 nM, as shown in Table 4-8.

TABLE 4-1
Binding Kinetics parameters of anti-IL-36R
antibodies binding to hIL-36R-MMH at 25° C.

| | mAb Capture | 100 nM hIL-36R- | kₐ | k_{d} | K_{D} | t_{1/2} |
|---|---|---|---|---|---|---|
| Antibody | Level (RU) | MMH Bound (RU) | (1/Ms) | (1/s) | (M) | (min) |
| H4H14699P2 | 198 ± 0.6 | 74 | 1.65E+05 | 1.68E−03 | 1.02E−08 | 7 |
| H4H14700P2 | 159 ± 0.3 | 67 | 1.20E+05 | 5.79E−04 | 4.82E−09 | 20 |
| H4H14706P2 | 199 ± 0.5 | 89 | 1.21E+05 | 4.95E−04 | 4.08E−09 | 23 |
| H4H14708P2 | 209 ± 0.9 | 76 | 9.14E+04 | 6.23E−04 | 6.82E−09 | 19 |
| H4H14709P | 156 ± 0.2 | 64 | 7.23E+04 | 2.96E−04 | 4.09E−09 | 39 |
| H4H14728P | 175 ± 0.6 | 69 | 983E+04 | 7.73E−04 | 7.87E−09 | 15 |
| H4H14731P | 204 ± 0.6 | 54 | 9.22E+04 | 3.43E−04 | 3.72E−09 | 34 |
| H4H14732P2 | 197 ± 0.3 | 26 | 4.10E+04 | 5.69E−04 | 1.39E−08 | 20 |
| H4H14734P2 | 174 ± 0.8 | 22 | 3.32E+04 | 4.04E−04 | 1.22E−08 | 29 |
| H4H14757P | 180 ± 0.9 | 94 | 1.82E+05 | 3.96E−04 | 2.18E−09 | 29 |
| H4H14758P | 177 ± 0.7 | 87 | 1.21E+05 | 9.23E−04 | 7.63E−09 | 13 |
| H4H14760P2 | 180 ± 0.5 | 61 | 6.79E+04 | 4.15E−04 | 6.11E−09 | 28 |

TABLE 4-2
Binding Kinetics parameters of anti-IL-36R
antibodies binding to hIL-36R-MMH at 37° C.

| | mAb Capture | 100 nM hIL-36R-MMH | kₐ | k_{d} | K_{D} | t_{1/2} |
|---|---|---|---|---|---|---|
| Antibody | Level (RU) | Bound (RU) | (1/Ms) | (1/s) | (M) | (min) |
| H4H14699P2 | 257 ± 1.2 | 89 | 1.42E+05 | 4.18E−03 | 2.95E−08 | 2.8 |
| H4H14700P2 | 218 ± 0.7 | 89 | 1.76E+05 | 1.84E−03 | 1.05E−08 | 6 |
| H4H14706P2 | 266 ± 1 | 113 | 1.50E+05 | 1.30E−03 | 8.65E−09 | 9 |
| H4H14708P2 | 280 ± 2.5 | 106 | 1.36E+05 | 1.86E−03 | 1.36E−08 | 6 |
| H4H14709P | 218 ± 0.9 | 106 | 1.31E+05 | 5.54E−04 | 4.25E−09 | 21 |
| H4H14728P | 242 ± 0.7 | 93 | 1.36E+05 | 2.93E−03 | 2.15E−08 | 4 |
| H4H14731P | 262 ± 0.7 | 81 | 1.37E+05 | 9.71E−04 | 7.11E−09 | 12 |
| H4H14732P2 | 272 ± 0.8 | 34 | 4.21E+04 | 1.16E−03 | 2.76E−08 | 10 |
| H4H14734P2 | 248 ± 0.8 | 25 | 5.39E+04 | 7.49E−04 | 1.39E−08 | 15 |
| H4H14757P | 262 ± 1.0 | 129 | 2.10E+05 | 1.09E−03 | 5.18E−09 | 11 |
| H4H14758P | 247 ± 1.2 | 111 | 1.58E+05 | 2.37E−03 | 1.50E−08 | 5 |
| H4H14760P2 | 252 ± 0.8 | 83 | 9.06E+04 | 2.08E−03 | 2.30E−08 | 6 |

TABLE 4-3
Binding Kinetics parameters of anti-IL-36R
antibodies binding to mfIL-36R-MMH at 25° C.

| | mAb Capture | 100 nM hIL-36R-MMH | kₐ | k_{d} | K_{D} | t_{1/2} |
|---|---|---|---|---|---|---|
| Antibody | Level (RU) | Bound (RU) | (1/Ms) | (1/s) | (M) | (min) |
| H4H4699P2 | 198 ± 0.4 | 53 | 6.38E+04 | 1.83E−03 | 2.87E−08 | 6 |
| H4H14700P2 | 158 ± 0.3 | 48 | 6.02E+04 | 6.35E−04 | 1.06E−08 | 18 |
| H4H14706P2 | 199 ± 0.4 | 65 | 6.72E+04 | 5.52E−04 | 8.21E−09 | 21 |
| H4H14708P2 | 209 ± 0.9 | 51 | 4.50E+04 | 5.79E−04 | 1.29E−08 | 20 |
| H4H14709P | 156 ± 0.3 | 34 | 2.80E+04 | 4.01E−04 | 1.43E−08 | 29 |
| H4H14728P | 175 ± 0.6 | 51 | 5.15E+04 | 4.06E−04 | 7.87E−09 | 28 |
| H4H14731P | 203 ± 0.4 | 33 | 5.98E+04 | 9.51E−04 | 1.59E−08 | 12 |
| H4H14732P2 | 197 ± 0.4 | 12 | 1.88E+04 | 6.49E−04 | 3.44E−08 | 18 |
| H4H14734P2 | 175 ± 0.6 | 12 | 2.60E+04 | 2.54E−04 | 9.78E−09 | 46 |
| H4H14757P | 183 ± 0.7 | 1 | NB* | NB* | NB* | NB* |
| H4H14758P | 179 ± 0.6 | −1 | NB* | NB* | NB* | NB* |
| H4H14760P2 | 181 ± 0.4 | 0 | NB* | NB* | NB* | NB* |

*NB indicates that under the experimental conditions, mfIL-36R-MMH reagent did not bind to the captured anti-IL-36R antibody

TABLE 4-4
Binding Kinetics parameters of anti-IL-36R
antibodies binding to mfIL-36R-MMH at 37° C.

| | | 100 nM | | | | |
|---|---|---|---|---|---|---|
| | mAb Capture | mfIL-36R-MMH | kₐ | k_{d} | K_{D} | t_{1/2} |
| Antibody | Level (RU) | Bound (RU) | (1/Ms) | (1/s) | (M) | (min) |
| H4H14699P2 | 257 ± 0.4 | 59 | 7.80E+04 | 4.54E−03 | 5.82E−08 | 2.5 |
| H4H14700P2 | 218 ± 0.7 | 67 | 7.14E+04 | 1.98E−03 | 2.78E−08 | 6 |
| H4H14706P2 | 266 ± 0.4 | 84 | 7.47E+04 | 1.42E−03 | 1.90E−08 | 8 |
| H4H14708P2 | 279 ± 2.8 | 75 | 5.96E+04 | 1.81E−03 | 3.04E−08 | 6 |
| H4H14709P | 220 ± 1.4 | 66 | 4.91E+04 | 8.71E−04 | 1.77E−08 | 13 |
| H4H14728P | 243 ± 0.7 | 77 | 6.48E+04 | 1.34E−03 | 2.07E−08 | 9 |
| H4H14731P | 261 ± 0.3 | 41 | 6.68E+04 | 3.22E−03 | 4.82E−08 | 4 |
| H4H14732P2 | 273 ± 0.8 | 17 | 3.19E+04 | 1.64E−03 | 5.15E−08 | 7 |
| H4H14734P2 | 248 ± 0.6 | 12 | 3.61E+04 | 5.21E−04 | 1.44E−08 | 22 |
| H4H14757P | 264 ± 1.5 | 4 | NB* | NB* | NB* | NB* |
| H4H14758P | 248 ± 0.9 | −1 | NB* | NB* | NB* | NB* |
| H4H14760P2 | 253 ± 0.9 | 2 | NB* | NB* | NB* | NB* |

*NB indicates that under the experimental conditions, mfIL-36R-MMH reagent did not bind to the captured anti-IL-36R antibody

TABLE 4-5
Binding Kinetics parameters of anti-IL-36R
antibodies binding to hIL-36R-mFc at 25° C.

| | mAb Capture | 100 nM hIL-36R-mFc | kₐ | k_{d} | K_{D} | t_{1/2} |
|---|---|---|---|---|---|---|
| Antibody | Level (RU) | Bound (RU) | (1/Ms) | (1/s) | (M) | (min) |
| H4H14699P2 | 197 ± 0.8 | 150 | 5.64E+05 | 2.03E−04 | 3.59E−10 | 57 |
| H4H14700P2 | 158 ± 0.3 | 128 | 5.17E+05 | 1.20E−04 | 2.33E−10 | 96 |
| H4H14706P2 | 197 ± 1.7 | 163 | 5.75E+05 | 1.32E−04 | 2.30E−10 | 87 |
| H4H14708P2 | 207 ± 2.3 | 146 | 4.03E+05 | 1.28E−04 | 3.17E−10 | 90 |
| H4H14709P | 155 ± 0.7 | 142 | 2.57E+05 | 8.62E−05 | 3.35E−10 | 134 |
| H4H14728P | 174 ± 0.5 | 7 | IC* | IC* | IC* | IC* |
| H4H14731P | 204 ± 0.3 | 10 | 5.58E+04 | 3.23E−04 | 5.79E−09 | 36 |
| H4H14732P2 | 197 ± 0.6 | 145 | 5.70E+05 | 7.97E−04 | 1.40E−09 | 14 |
| H4H14734P2 | 174 ± 0.5 | 77 | 6.22E+04 | 1.01E−04 | 1.63E−09 | 114 |
| H4H14757P | 182 ± 1.4 | 167 | 5.75E+05 | 9.97E−05 | 1.73E−10 | 116 |
| H4H14758P | 179 ± 0.8 | 161 | 5.26E+05 | 1.63E−04 | 3.09E−10 | 71 |
| H4H14760P2 | 181 ± 0.8 | 121 | 2.07E+05 | 1.09E−04 | 5.26E−10 | 106 |

*IC indicates that under the experimental conditions, hIL-36R.mFc binding is inconclusive

TABLE 4-6
Binding Kinetics parameters of anti-IL-36R
antibodies binding to hIL-36R-mFc at 37° C.

| | mAb Capture | 100 nM hIL-36R-mFc | kₐ | k_{d} | K_{D} | t_{1/2} |
|---|---|---|---|---|---|---|
| Antibody | Level (RU) | Bound (RU) | (1/Ms) | (1/s) | (M) | (min) |
| H4H14699P2 | 258 ± 0.7 | 186 | 5.94E+05 | 4.56E−04 | 7.67E−10 | 25 |
| H4H14700P2 | 218 ± 0.5 | 174 | 5.35E+05 | 2.09E−04 | 3.90E−10 | 55 |
| H4H14706P2 | 266 ± 0.6 | 207 | 5.93E+05 | 2.66E−04 | 4.49E−10 | 43 |
| H4H14708P2 | 280 ± 2.5 | 203 | 4.60E+05 | 2.00E−04 | 4.35E−10 | 58 |
| H4H14709P | 218 ± 1.2 | 211 | 5.43E+05 | 1.12E−04 | 2.05E−10 | 104 |
| H4H14728P | 243 ± 1.0 | 11 | 1.62E+04 | 4.65E−04 | 2.87E−08 | 25 |
| H4H14731P | 261 ± 0.5 | 12 | 6.99E+04 | 4.50E−04 | 6.43E−09 | 26 |
| H4H14732P2 | 273 ± 1.2 | 195 | 6.35E+05 | 1.27E−03 | 2.00E−09 | 9 |
| H4H14734P2 | 247 ± 1.0 | 96 | 5.27E+04 | 1.22E−04 | 2.31E−09 | 95 |
| H4H14757P | 264 ± 2.0 | 235 | 6.16E+05 | 1.50E−04 | 2.43E−10 | 77 |
| H4H14758P | 248 ± 0.7 | 210 | 5.54E+05 | 2.86E−04 | 5.17E−10 | 40 |
| H4H14760P2 | 254 ± 0.9 | 173 | 2.17E+05 | 2.22E−04 | 1.02E−09 | 52 |

TABLE 4-7
Binding Kinetics parameters of anti-IL-36R antibodies
binding to hIL-36R-Trap-mFc at 25° C.

| | mAb Capture | 100 nM hIL-36R-mFc | kₐ | k_{d} | K_{D} | t_{1/2} |
|---|---|---|---|---|---|---|
| Antibody | Level (RU) | Bound (RU) | (1/Ms) | (1/s) | (M) | (min) |
| H4H14699P2 | 196 ± 0.7 | 188 | 4.98E+05 | 2.28E04 | 4.58E−10 | 51 |
| H4H14700P2 | 156 ± 0.7 | 157 | 4.42E+05 | 1.25E−04 | 2.83E−10 | 92 |
| H4H14706P2 | 195 ± 0.6 | 205 | 4.77E+05 | 1.10E−04 | 2.32E−10 | 105 |
| H4H14708P2 | 205 ± 2.1 | 172 | 3.61E+05 | 1.29E−04 | 3.57E−10 | 90 |
| H4H14709P | 155 ± 0.4 | 173 | 2.00E+05 | 8.07E−05 | 4.04E−10 | 143 |
| H4H14728P | 175 ± 0.5 | 63 | 4.03E+04 | 5.65E−04 | 1.40E−08 | 20 |
| H4H14731P | 203 ± 0.5 | 60 | 4.52E+04 | 2.01E−04 | 4.45E−09 | 57 |
| H4H14732P2 | 197 ± 0.4 | 161 | 4.62E+05 | 1.36E−03 | 2.95E−09 | 8 |
| H4H14734P2 | 174 ± 0.5 | 85 | 4.89E+04 | 1.10E−04 | 2.24E−09 | 105 |
| H4H14757P | 181 ± 0.5 | 202 | 4.85E+05 | 1.03E−04 | 2.12E−10 | 113 |
| H4H14758P | 179 ± 0.6 | 197 | 4.36E+05 | 1.80E−04 | 4.13E−10 | 64 |
| H4H14760P2 | 181 ± 1.0 | 134 | 1.58E+05 | 1.00E−04 | 6.36E−10 | 115 |

TABLE 4-8
Binding Kinetics parameters of anti-IL-36R antibodies binding to
hIL-36R-Trap-mFc at 37° C.

| | | 100 nM | | | | |
|---|---|---|---|---|---|---|
| | mAb | hIL-36R- | | | | |
| | Capture | Trap-mFc | | | | |
| | Level | Bound | kₐ | k_{d} | K_{D} | t½ |
| Antibody | (RU) | (RU) | (1/Ms) | (1/s) | (M) | (min) |
| H4H14699P2 | 256 ± 0.8 | 229 | 4.84E+05 | 5.13E−04 | 1.06E−09 | 23 |
| H4H14700P2 | 217 ± 0.5 | 216 | 4.55E+05 | 2.53E−04 | 5.55E−10 | 46 |
| H4H14706P2 | 266 ± 0.8 | 264 | 4.91E+05 | 2.71E−04 | 5.51E−10 | 43 |
| H4H14708P2 | 280 ± 1 | 239 | 3.85E+05 | 2.53E−04 | 6.56E−10 | 46 |
| H4H14709P | 218 ± 1.3 | 257 | 4.67E+05 | 1.23E−04 | 2.64E−10 | 94 |
| H4H14728P | 243 ± 0.5 | 89 | 4.85E+04 | 1.98E−03 | 4.09E−08 | 6 |
| H4H14731P | 261 ± 0.4 | 78 | 5.26E+04 | 5.14E−04 | 9.77E−09 | 22 |
| H4H14732P2 | 272 ± 01 | 212 | 5.01E+05 | 1.82E−03 | 3.63E−09 | 6 |
| H4H14734P2 | 248 ± 0.5 | 99 | 4.84E+04 | 1.43E−04 | 2.96E−09 | 81 |
| H4H14757P | 263 ± 1.2 | 281 | 5.23E+05 | 1.96E−04 | 3.74E−10 | 59 |
| H4H14758P | 248 ± 0.6 | 251 | 4.68E+05 | 3.63E−04 | 7.77E−10 | 32 |
| H4H14760P2 | 254 ± 1.1 | 195 | 1.66E+05 | 2.83E−04 | 1.70E−09 | 41 |

Additional binding experiments were performed to determine the effect of pH on the rate of dissociation of IL-36R bound to purified anti-IL-36R antibodies, which was determined using a real-time surface plasmon resonance biosensor using a Biacore T200 instrument. The Biacore sensor surface was first derivatized by amine coupling with a monoclonal mouse anti-human Fc antibody (GE, #BR-1008-39) to capture anti-IL-36R antibodies. These Biacore binding studies were performed using two running buffers PBS-T, pH7.4 (8.1 mM Na₂HPO₄, 1.9 mM NaH₂PO₄, 3 mM KCl, 137 mM NaCl, 0.05% v/v Tween-20, adjusted to pH 7.4) and PBS-T, pH 6.0 (6.6 mM Na₂HPO₄, 3.4 mM NaH₂PO₄, 3 mM KCl, 137 mM NaCl, 0.05% v/v Tween-20, adjusted to pH6.0). Different concentrations of hIL-36R-MMH and mfIL-36R-MMH prepared in PBS-T, pH7.4 buffer (ranging from 100 nM to 11.11 nM, 3-fold dilutions) were injected over the anti-IL-36R antibody captured surface for 4 minutes at a flow rate of 50 μL/minute and their dissociation in two running buffers, PBS-T, pH7.4 and PBS-T, pH 6.0, was monitored for 10 minutes. All of these binding kinetics experiments were performed at 25° C. and 37° C. Kinetic dissociation constant (k_{d}) were determined by fitting the real-time sensorgrams to a 1:1 binding model using Scrubber 2.0c curve fitting software. Binding dissociative half-lives (t½) were calculated from k_{d} as:

$$t \frac{1}{2} ( min ) = \frac{ln ( 2 )}{60 * kd}$$

Binding dissociation rate constants for hIL-36R-MMH or mfIL-36R-MMH binding to different anti-IL-36R antibodies at 25° C. and 37° C. in two running buffers PBS-T, pH7.4 and PBS-T, pH 6.0 are shown in Tables 4-9 through 4-12.

TABLE 4-9
Binding dissociation rate constant of anti-IL-36R monoclonal antibodies
binding to hIL-36R-MMH in two running buffers performed at 25° C.

| | PBS-T, pH 7.4 Running Buffer | | | | PBS-T, pH 6.0 Running Buffer | | | |
|---|---|---|---|---|---|---|---|---|
| | | 100 nM | | | | 100 nM | | |
| | | Human | | | | Human | | |
| | mAb | IL-36R- | | | mAb | IL-36R- | | |
| | Capture | MMH | | | Capture | MMH | | |
| | Level | Bound | k_{d} | t½ | Level | Bound | k_{d} | t½ |
| mAb PID | (RU) | (RU) | (1/s) | (min) | (RU) | (RU) | (1/s) | (min) |
| H4H14699P2 | 255 ± 2.7 | 84 | 1.93E−03 | 6 | 278 ± 0.6 | 79 | 2.93E−03 | 4 |
| H4H14700P2 | 330 ± 0.9 | 128 | 7.95E−04 | 15 | 350 ± 0.9 | 120 | 1.72E−03 | 7 |
| H4H14706P2 | 298 ± 0.9 | 119 | 6.16E−04 | 19 | 312 ± 2.2 | 109 | 9.27E−04 | 12 |
| H4H14708P2 | 268 ± 2.8 | 87 | 7.96E−04 | 15 | 283 ± 2.5 | 79 | 1.63E−03 | 7 |
| H4H14709P | 283 ± 0.8 | 111 | 4.20E−04 | 28 | 300 ± 1.8 | 99 | 8.42E−04 | 14 |
| H4H14728P | 265 ± 1.7 | 103 | 8.98E−04 | 13 | 269 ± 2 | 96 | 8.87E−04 | 13 |
| H4H14731P | 282 ± 1.9 | 65 | 5.53E−04 | 21 | 281 ± 0.9 | 43 | 6.79E−04 | 17 |
| H4H14732P2 | 244 ± 1.5 | 32 | 8.65E−04 | 13 | 255 ± 1.3 | 28 | 9.18E−04 | 13 |
| H4H14734P2 | 230 ± 1.3 | 20 | 7.38E−04 | 16 | 240 ± 1.8 | 18 | 7.87E−04 | 15 |
| H4H14757P | 226 ± 0.6 | 105 | 5.97E−04 | 19 | 235 ± 1.5 | 98 | 8.87E−04 | 13 |
| H4H14758P | 244 ± 2.5 | 108 | 1.12E−03 | 10 | 255 ± 1.6 | 103 | 1.80E−03 | 6 |
| H4H14760P2 | 257 ± 1.5 | 80 | 5.45E−04 | 21 | 266 ± 1.3 | 69 | 9.72E−04 | 12 |

TABLE 4-10
Binding dissociation rate constant of anti-IL-36R monoclonal antibodies
binding to hIL-36R-MMH in two running buffers performed at 37° C.

| | PBS-T, pH 7.4 Running Buffer | | | | PBS-T, pH 6.0 Running Buffer | | | |
|---|---|---|---|---|---|---|---|---|
| | | 100 nM | | | | 100 nM | | |
| | mAb | hIL-36R- | | | mAb | hIL-36R- | | |
| | Capture | MMH | | | Capture | MMH | | |
| | Level | Bound | k_{d} | t½ | Level | Bound | k_{d} | t½ |
| mAb PID | (RU) | (RU) | (1/s) | (min) | (RU) | (RU) | (1/s) | (min) |
| H4H14699P2 | 312 ± 2.9 | 96 | 4.15E−03 | 3 | 300 ± 14.2 | 83 | 5.81E−03 | 2 |
| H4H14700P2 | 422 ± 5.7 | 153 | 1.69E−03 | 7 | 435 ± 1.5 | 144 | 3.50E−03 | 3 |
| H4H14706P2 | 367 ± 3.5 | 140 | 1.31E−03 | 9 | 378 ± 2 | 132 | 2.33E−03 | 5 |
| H4H14708P2 | 313 ± 6.8 | 105 | 1.77E−03 | 7 | 318 ± 4.6 | 95 | 3.37E−03 | 3 |
| H4H14709P | 372 ± 3 | 159 | 7.04E−04 | 16 | 380 ± 2.9 | 146 | 1.72E−03 | 7 |
| H4H14728P | 306 ± 1 | 121 | 2.96E−03 | 4 | 302 ± 1.3 | 114 | 3.06E−03 | 4 |
| H4H14731P | 272 ± 3.9 | 91 | 1.08E−03 | 11 | 276 ± 1.6 | 84 | 2.01E−03 | 6 |
| H4H14732P2 | 303 ± 3 | 40 | 1.10E−03 | 10 | 310 ± 2 | 36 | 1.23E−03 | 9 |
| H4H14734P2 | 287 ± 1.4 | 20 | 1.21E−03 | 10 | 289 ± 1.8 | 17 | 1.69E−03 | 7 |
| H4H14757P | 254 ± 0.7 | 113 | 1.29E−03 | 9 | 267 ± 1 | 109 | 2.60E−03 | 4 |
| H4H14758P | 308 ± 1.2 | 126 | 2.26E−03 | 5 | 314 ± 0.5 | 120 | 3.39E−03 | 3 |
| H4H14760P2 | 311 ± 1.4 | 94 | 1.87E−03 | 6 | 317 ± 2.1 | 85 | 3.90E−03 | 3 |

TABLE 4-11
Binding dissociation rate constant of anti-IL-36R monoclonal antibodies
binding to mfIL-36R-MMH in two running buffers performed at 25° C.

| | PBS-T, pH 7.4 Running Buffer | | | | PBS-T, pH 6.0 Running Buffer | | | |
|---|---|---|---|---|---|---|---|---|
| | | 100 nM | | | | 100 nM | | |
| | | Human | | | | Human | | |
| | mAb | mfIL-36R- | | | mAb | mfIL-36R- | | |
| | Capture | MMH | | | Capture | MMH | | |
| | Level | Bound | k_{d} | t½ | Level | Bound | k_{d} | t½ |
| mAb PID | (RU) | (RU) | (1/s) | (min) | (RU) | (RU) | (1/s) | (min) |
| H4H14699P2 | 258 ± 1.5 | 55 | 2.04E−03 | 6 | 276 ± 0.7 | 48 | 3.22E−03 | 4 |
| H4H14700P2 | 331 ± 1.8 | 91 | 8.21E−04 | 14 | 350 ± 1.7 | 80 | 1.71E−03 | 7 |
| H4H14706P2 | 295 ± 1.7 | 80 | 6.46E−04 | 18 | 312 ± 1.5 | 71 | 9.65E−04 | 12 |
| H4H14708P2 | 270 ± 2 | 57 | 7.52E−04 | 15 | 281 ± 1.2 | 47 | 1.50E−03 | 8 |
| H4H14709P | 282 ± 1.3 | 57 | 5.19E−04 | 22 | 301 ± 0.7 | 48 | 1.12E−03 | 10 |
| H4H14728P | 264 ± 2 | 74 | 5.44E−04 | 21 | 269 ± 1.2 | 68 | 6.16E−04 | 19 |
| H4H14731P | 279 ± 2.2 | 36 | 1.37E−03 | 8 | 279 ± 1.9 | 23 | 1.52E−03 | 8 |
| H4H14732P2 | 245 ± 0.9 | 14 | 7.87E−04 | 15 | 253 ± 0.9 | 12 | 1.15E−03 | 10 |
| H4H14734P2 | 229 ± 2.2 | 9 | 5.05E−04 | 23 | 238 ± 1.2 | 8 | 6.31E−04 | 18 |
| H4H14757P | 224 ± 1.8 | 1 | NB* | NB* | 235 ± 0.9 | 1 | NB* | NB* |
| H4H14758P | 243 ± 0.5 | 0 | NB* | NB* | 254 ± 1 | 1 | NB* | NB* |
| H4H14760P2 | 257 ± 1.9 | 1 | NB* | NB* | 266 ± 1.2 | 1 | NB* | NB* |

*NB indicates that under the current experimental conditions, no binding of mfIL-36R-MMH to anti-hFc captured anti-IL-36R mAb was observed.

TABLE 4-12
Binding dissociation rate constant of anti-IL-36R monoclonal antibodies
binding to mfIL-36R-MMIA in two running buffers performed at 37° C.

| | PBS-T, pH 7.4 Running Buffer | | | | PBS-T, pH 6.0 Running Buffer | | | |
|---|---|---|---|---|---|---|---|---|
| | | 100 nM | | | | 100 nM | | |
| | | Human | | | | Human | | |
| | mAb | mfIL-36R- | | | mAb | mfIL-36R- | | |
| | Capture | MMH | | | Capture | MMH | | |
| | Level | Bound | kd | t½ | Level | Bound | kd | t½ |
| mAb PID | (RU) | (RU) | (1/s) | (min) | (RU) | (RU) | (1/s) | (min) |
| H4H14699P2 | 310 ± 4.9 | 58 | 4.59E−03 | 3 | 308 ± 1.9 | 50 | 6.22E−03 | 2 |
| H4H14700P2 | 422 ± 1.1 | 108 | 1.80E−03 | 6 | 434 ± 1.8 | 97 | 3.33E−03 | 3 |
| H4H14706P2 | 366 ± 1.3 | 95 | 1.38E−03 | 8 | 375 ± 1.6 | 85 | 2.64E−03 | 4 |
| H4H14708P2 | 302 ± 3.6 | 66 | 1.69E−03 | 7 | 314 ± 2.2 | 58 | 3.10E−03 | 4 |
| H4H14709P | 370 ± 2.3 | 92 | 9.87E−04 | 12 | 379 ± 1 | 80 | 2.94E−03 | 4 |
| H4H14728P | 305 ± 2.5 | 98 | 1.39E−03 | 8 | 301 ± 1.2 | 91 | 1.91E−03 | 6 |
| H4H14731P | 266 ± 4 | 43 | 3.61E−03 | 3 | 279 ± 1.8 | 40 | 5.38E−03 | 2 |
| H4H14732P2 | 302 ± 1.4 | 18 | 9.37E−04 | 12 | 309 ± 1.7 | 16 | 1.57E−03 | 7 |
| H4H14734P2 | 283 ± 0.8 | 9 | 7.87E−04 | 15 | 287 ± 1.9 | 7 | 1.19E−03 | 10 |
| H4H14757P | 255 ± 0.5 | 0 | NB* | NB* | 267 ± 1.9 | −1 | NB* | NB* |
| H4H14758P | 306 ± 1 | 0 | NB* | NB* | 314 ± 2.5 | 1 | NB* | NB* |
| H4H14760P2 | 309 ± 1.6 | 1 | NB* | NB* | 315 ± 1.3 | 1 | NB* | NB* |

*NB indicates that under the current experimental conditions, no binding of mfIL-36R-MMH to anti-hFc captured anti-IL-36R mAb was observed

### Example 5: In Vivo Evaluation of Anti-IL36R in IMQ-Induced Skin Inflammation and Chronic Colitis Mouse Models

The anti-human IL-36R monoclonal antibodies of the present invention were tested in vivo in acute and chronic Imiquimod (IMQ)-induced skin inflammation, and chronic dextran sodium sulfate (DSS)-induced colitis in humanized IL-36R/hIL-36α, J, 7 mice. Cytokine detection was performed in skin and colon homogenates using a proinflammatory panel 1 (mouse) multiplex immunoassay kit. Detection of Lipocalin 2 (Lcn2) in fecal homogenates was performed using a mouse Duoset Lipocalin-2/NGAL ELISA kit. Measurement of myeloperoxidase (MPO) activity in the colon homogenates was done using a mouse MPO ELISA kit.

The anti-IL36R antibodies, H4H14706P2 and H4H14708P2, were used along with a human, isotype matched control IgG4 antibody.

To examine the role of IL-36R in skin and intestinal inflammation and to test the efficacy of hIL-36R antagonism in vivo, anti-human IL-36R monoclonal antibodies of the present invention were tested in the murine models of Imiquimod (IMQ)-induced skin inflammation and DSS-induced chronic colitis. In both models, Velocigene generated homozygous mice expressing human IL-36R and human IL-36α, J, 7 and endogenous mouse IL-36Ra were utilized (resulting mice are referred to as DITRA-like mice, due to decreased affinity of mouse IL-36Ra for human IL-36R which resembles the mutation observed in DITRA (Deficiency of Interleukin Thirty-six Receptor Antagonist) patients (Marrakchi et al., Interleukin-36-receptor antagonist deficiency and generalized pustular psoriasis, N Engl J Med 365:620-628 (2011)).

A mouse humanized strain with the genotype Il1r12^{hu/hu} Il1f6^{hu/hu} Il1f8^{hu/hu} Il1f9^{hu/hu} was generated. In this mouse strain, human IL1F6, IL1F8, and IL1F9 replaced the endogenous mouse IL1F6, IL1F8, and IL1F9 (also called IL36α, β and γ respectively); and a chimeric IL1RL2 replaced the endogenous mouse IL1RL2. The chimeric IL1RL2 had a human IL1RL2 extracellular domain and a mouse intracellular domain. This resulted in a chimeric receptor that maintained the intracellular signaling specificity of mice, while rendering the extracellular domain human and, thus, able to bind to the human ligands IL1F6, IL1F8, and IL1F9.

Acute and chronic IMQ-induced skin inflammation induction and antibody treatment in DITRA-like mice. To induce skin inflammation, 8-10 weeks old humanized DITRA-like female mice had their back hair shaved using mouse hair trimmer (Oster, MiniMax, Cat #78049-100) and skin depilated with 0.5 g Veet hair removal gel three days prior to IMQ cream application. A daily topical dose of 62.5 mg of commercially available IMQ cream (5%) (Aldara, GM Health Care Limited, NDC 99207-206-12) or Vaseline (CVS Pharmacy) was applied on the shaved back skin of the mice for four consecutive days for acute and nine days for chronic disease induction. A daily topical dose of 62.5 mg of Aldara translated into a daily dose of 3.125 mg of an active compound. In acute IMQ-induced skin inflammation, anti-human IL-36R antibodies, H4H14706P2 and H4H14708P2, were administered subcutaneously into back skin at 10 mg/kg and 1 mg/kg on three days before (−3d) and one day after (d1) starting the IMQ application. Control group received PBS and 10 mg/kg of hIgG4 Isotype control injections. In chronic IMQ-induced skin inflammation, anti-human IL-36R antibodies, H4H14706P2 and H4H14708P2, were administered subcutaneously into back skin at 10 mg/kg therapeutically on d4 and d8. Two or three days after the start of IMQ application, the back skin of the mice started to display signs of erythema, scaling and thickening. The severity of inflammation was measured on a daily basis using an adapted version of the clinical Psoriasis Area and Severity Index. Erythema, scaling and thickening were scored independently on a scale from 0-4: 0, none; 1, slight; 2, moderate; 3, marked; and 4, very marked (van der Fits et al., Imiquimod-induced psoriasis-like skin inflammation in mice is mediated via the IL-23/IL-17 axis. J Immunol 2009, 182:5836-5845). On d4 of acute and d11 of chronic IMQ-induced skin inflammation, skin thickness was measured using a caliper (Kaefer).

Histopathology. Skin tissues of 6 mm diameter from murine back were fixed in 10% buffered formalin, and 4-5 μm paraffin embedded sections were stained with hematoxylin and eosin. Skin sections were evaluated blindly for the presence of parakeratosis, orthokeratosis, Munro's microabscess, acanthosis, epidermal ulceration, inflammation in the dermis and hypodermis, blood vessel congestion in the dermis and hypodermis, follicular hyperkeratosis and epithelial hyperplasia. A 0-4 scoring scale was used: 0-within normal limits, 1-minimal, 2-mild, 3-moderate and 4-severe. A total pathology score was calculated for each mouse by adding the individual histopathological feature scores. Data analysis was performed using GraphPad Prism™ software. Danilenko, Review paper: preclinical models of psoriasis, Vet Pathol. 2008 July; 45(4):563-75; Lowes et al., Pathogenesis and therapy of psoriasis, Nature. 2007 Feb. 22; 445(7130):866-73; Mecklenburg et al., Proliferative and non-proliferative lesions of the rat and mouse integument, J Toxicol Pathol. 2013;26(3 Suppl):27S-57S; Uribe-Herranz et al., IL-1R1 signaling facilitates Munro's microabscess formation in psoriasiform imiquimod-induced skin inflammation, J Invest Dermatol. 2013 June; 133(6):1541-9; van der Fits et al., Imiquimod-induced psoriasis-like skin inflammation in mice is mediated via the IL-23/IL-17 axis, J Immunol. 2009 May 1;182(9):5836-45.

Measurement of cytokines in skin homogenates. Full thickness skin tissues of 6 mm diameter from murine back were taken and placed in 15 mL tube containing T-per buffer (Thermo Scientific, Cat #378510), 1× Halt Protease Inhibitor Cocktail (Thermo Scientific, Cat #87786) and 5 M EDTA Solution (Thermo Scientific, Cat3 78429). Skin tissues were disrupted at 28000 rpm for 1 minute using a Polytron (PT10-35 GT-D, Cat #9158158) and put on ice. Generated skin homogenates were centrifuged at 1500 rpm for 8 minutes at 4° C. and the supernatants were collected into 96-well plates. Skin homogenates were subjected to Bradford protein assay using protein assay dye (BioRad, Cat #500-0006) to quantify the total protein content. Cytokine concentrations in the skin homogenates were measured using a Proinflammatory Panel 1 (mouse) multiplex immunoassay kit (MesoScale Discovery, Cat #K15048D) according to manufacturer's instructions. In brief, 50 μL/well of calibrators and samples (diluted in Diluent 41) were added to the plates pre-coated with capture antibodies and incubated at room temperature while shaking at 700 rpm for 2 hours. The plates were then washed 3 times with 1×PBS containing 0.05% (w/v) Tween-20, followed by the addition of 25 μL of Detection Antibody Solution diluted in Diluent 45. After 2-hours incubation at room temperature while shaking, the plates were washed 3 times, and 150 μL of 2× Read Buffer was added to each well. Electrochemiluminescence was immediately read on a MSD Spector® instrument. Data analysis was performed using GraphPad Prism™ software. Cytokine levels were normalized to total protein content.

Induction of DSS-induced model of chronic colitis and antibody treatment in DITRA-like mice. To induce chronic DSS-mediated colitis, female DITRA-like mice aged 12-20 weeks with an average body weight of more than 23 g were given 3% DSS (Sigma-Aldrich Cat #87786) in drinking water for 7 days followed by distilled water for 10 days. This cycle was repeated two times until d28. Control group received distilled water for the duration of the study. Anti-human IL-36R antibodies, H4H14706P2 and H4H14708P2, were administered intraperitoneally at 10 mg/kg and 5 mg/kg bi-weekly starting on d7. Control group received PBS and 10 mg/kg of hIgG4 Isotype control injections. Mice were weighted and monitored for clinical signs of colitis (e.g., stool consistency and fecal blood) on a daily basis. On d28, mice were euthanized and colon lengths were measured.

Measurement of Lcn-2 in colon homogenates. To monitor intestinal inflammation throughout the study, feces from individual DITRA-like mice were collected into 2 mL deep well plates on a weekly basis and stored at −80° C. Upon the completion of the study, feces collected on different days were subjected to homogenization. In brief, fecal samples were reconstituted with 1 mL PBS containing 0.1% Tween-20, 1× Halt Protease Inhibitor Cocktail (Thermo Scientific, Cat #87786) and 5 M EDTA Solution (Thermo Scientific, Cat3 78429). After adding 2 Tungsten 3 mm Carbide Beads to the wells (Qiagen, Cat #69997), the plates were placed on a shaker at highest speed overnight at 4° C. Homogenous fecal suspensions were centrifuged at 1200 rpm for 10 minutes at 4° C. and the supernatants were collected into 96-well plates. Fecal Lipocalin-2 (Lcn2) levels were measured using mouse Duoset Lipocalin-2/NGAL ELISA kit (R&D Systems, Cat #DY1857) according to manufacturer's instructions. Data analysis was performed using GraphPad Prism™ software.

Measurement of Myeloperoxidase (MPO) activity in colon homogenates. Pieces of the distal part of the colon were taken into 2 mL microcentrifuge tubes containing 2 Tungsten 3 mm Carbide Beads (Qiagen, Cat #69997) containing T-per buffer (Thermo Scientific, Cat #378510), 1× Halt Protease Inhibitor Cocktail (Thermo Scientific, Cat #87786) and 5 M EDTA Solution (Thermo Scientific, Cat #78429). Colon tissues were disrupted using Qiagen Tissue Lyser II at a frequency of 27.5 s⁻¹ for 10 minutes. Tubes were centrifuged at 1500 rpm for 8 minutes at 4° C. and the supernatants were collected into 96-well plates. Colon homogenates were subjected to Bradford protein assay using protein assay dye (BioRad, Cat #500-0006) to quantify the total protein content. Myeloperoxidase (MPO) activity in the colon homogenates was measured using mouse MPO ELISA Kit (Hycult Biotech, Cat #HK210-02) according to manufacturer's instructions. Data analysis was performed using GraphPad Prism™ software. MPO levels were normalized to total protein content.

Measurement of cytokines in colon homogenates. Cytokine concentrations in the colon homogenates were measured using a Proinflammatory Panel 1 (mouse) multiplex immunoassay kit (MesoScale Discovery, Cat #K15048D) according to manufacturer's instructions. In brief, 50 μL/well of calibrators and samples (diluted in Diluent 41) were added to the plates pre-coated with capture antibodies and incubated at room temperature while shaking at 700 rpm for 2 hours. The plates were then washed 3 times with 1×PBS containing 0.05% (w/v) Tween-20, followed by the addition of 25 μL of Detection Antibody Solution diluted in Diluent 45. After 2-hour incubation at room temperature while shaking, the plates were washed 3 times, and 150 μL of 2× Read Buffer was added to each well. Electrochemiluminescence was immediately read on a MSD Spector® instrument. Data analysis was performed using GraphPad Prism™ software. Cytokine levels were normalized to total protein content.

Statistical analysis. Statistical significance within the groups was determined by one-way Anova with Tukey's multiple comparison post-test (#, *p<0.01; ##, **p<0.001; ###, ***p<0.001; ####, ****p<0.0001).

### Results Summary and Conclusions

Anti-human IL-36R monoclonal antibodies inhibit acute skin inflammation in DITRA-like mice at prophylactic dosing. To examine the role of IL-36R in skin inflammation, two anti-human IL-36R monoclonal antibodies, H4H14706P2 and H4H14708P2, were tested in IMQ-induced model of psoriasiform dermititis that closely resembles human psoriasis lesions in terms of the phenotypic and histological characteristics (van der Fits et al., Imiquimod-induced psoriasis-like skin inflammation in mice is mediated via the IL-23/IL-17 axis, J Immunol 2009, 182:5836-5845; Swindell et al., Genome-wide expression profiling of five mouse models identifies similarities and differences with human psoriasis, PLoS One 2011, 6: e18266; Okayasu et al., A novel model in the induction of reliable experimental and chronic ulcerative colitis in mice, Gastroenterology 1990, 98:694-702). IMQ was applied daily to the shaved back skin of DITRA-like mice for four consecutive days. H4H14706P2 and H4H14708P2 antibodies were administered at 10 mg/kg and 1 mg/kg on −3d and d1. Control groups received PBS and hIgG4 Isotype control injections at 10 mg/kg. On d4, skin thickness was measured and tissue harvested for subsequent histopathological evaluation and protein isolation. Both H4H14706P2 and H4H14708P2 antibodies significantly decreased IMQ-induced skin thickness in a dose dependent manner compared to Isotype control (Table 5-1). Histopathological evaluation of the skin lesions revealed a significant reduction in total pathology score including parakeratosis and Munro's microabscess with anti-human IL-36R antibody treatment (Table 5-2).

TABLE 5-1
Anti-human IL-36R antibodies reduced skin thickness in acute IMQ-induced skin
inflammation. Thickness is presented in μm. Statistical significance within the groups was
determined by one-way Anova with Tukey's multiple comparison post-test and standard error
of mean (SEM±) calculated: ^{#}significantly different from Vaseline-treated group; *significantly
different from PBS- and Isotype-treated groups. N = 9/group.

| | IMQ | | | | | |
|---|---|---|---|---|---|---|
| Vaseline | | H4H14706P2 | | H4H14706132 | | hIgG4 Isotype |
| PBS | PBS | 1 mg/kg | 10 mg/kg | 1 mg/kg | 10 mg/kg | 10 mg/kg |
| 496.7 ± 8.8 | 825 ± 30^{####} | 674.4 ± 56*** | 546.7 ± 30.3**** | 624.4 ± 67*** | 586.7 ± 53**** | 822 ± 29.6^{####} |

p value:
^{#,} *p < 0.01;
^{##,} **p < 0.001;
^{###,} ***p < 0.001;
^{####,} ****p < 0.0001

TABLE 5-2
Anti-human IL-36R antibodies reduced skin thickness in acute IMQ-induced skin
inflammation. Statistical significance within the groups was determined by one-way Anova
with Tukey's multiple comparison post-test and standard error of mean (SEM±) calculated:
^{#}significantly different from Vaseline-treated group; *significantly different from PBS- and
Isotype-treated groups. N = 9/group.

| | IMQ | | | | | |
|---|---|---|---|---|---|---|
| Vaseline | | H4H14706P2 | | H4H14708P2 | | hIgG4 Isotype |
| PBS | PBS | 1 mg/kg | 10 rng/kg | 1 mg/kg | 10 mg/kg | 10 mg/kg |
| 0 | 20.3 ± 3^{####} | 17.1 ± 2.4 | 7 ± 3.6**** | 14.2 ± 2* | 8.9 ± 2.4**** | 20.6 ± 1.7^{####} |

p value:
^{#,} *p < 0.01;
^{##,} **p < 0.001;
^{###,} ***p < 0.001;
^{####,} ****p < 0.0001

Additionally, hIL-36R blockade, with H4H14706P2 and H4H14708P2 antibodies, resulted in 66-93% reduction in KC-GRO, IL-6, IL-1β and TNFα production in skin homogenates (Table 5-3).

TABLE 5-3
hIL-36R antagonism significantly reduced pro-inflammatory cytokines in IMQ-
treated skin of DITRA-like mice (acute skin inflammation model). Cytokine levels in
PBS/Vaseline control groups were subtracted from all the treatment groups. Statistical
significance within the groups was determined by one-way Anova with Tukey's multiple
comparison post-test and standard error of mean (SEM±) calculated: *significantly different
from PBS- and Isotype-treated groups. N = 9/group.

| Cytokines | IMQ | | | | | |
|---|---|---|---|---|---|---|
| (pg per mg of | | H4H14706P2 | | H4H14708P2 | | hIgG4 Isotype |
| total protein) | PBS | 1 mg/kg | 10 mg/kg | 1 mg/kg | 10 mg/kg | 10 mg/kg |
| KC-GRO | 122.5 ± 31.5 | 41.5 ± 12.4**** | 13.7 ± 4.3**** | 35.2 ± 16.7**** | 32.3 ± 23.5**** | 80.4 ± 12.9 |
| IL-6 | 134.8 ± 13 | 31.9 ± 12.4**** | 18.8 ± 8.4**** | 42.6 ± 17.7**** | 37.8 ± 26.9**** | 143.5 ± 57.5 |
| IL-1β | 84.4 ± 15.2 | 18.5 ± 10.1**** | 4.9 ± 3.7**** | 17.5 ± 13.8**** | 7.4 ± 5.6**** | 68.1 ± 15.1 |
| THF-α | 87.8 ± 6.5 | 23.6 ± 7.4**** | 8.2 ± 3.7**** | 18.9 ± 8**** | 9.5 ± 4.4**** | 80.3 ± 15.8 |

p value:
^{#,} *p < 0.01;
^{##,} **p < 0.001;
^{###,} ***p < 0.001;
^{####,} ****p < 0.0001

Anti-human IL-36R monoclonal antibodies inhibit chronic skin inflammation at therapeutic dosing. To further examine the therapeutic efficacy of hIL-36R antagonism in vivo, anti-human IL-36R antibodies were tested in chronic IMQ-induced model of skin inflammation. For the duration of two weeks, IMQ was applied to the shaved back skin of DITRA-like mice for nine days separated by two days without the application. H4H14706P2 and H4H14708P2 antibodies were administered subcutaneously at d4 and d8 at 10 mg/kg dose. Control groups received PBS and hIgG4 Isotype control injections at 10 mg/kg. On d11 skin thickness was measured and tissue harvested for subsequent histopathological evaluation and protein isolation. As shown in Tables 5-4 and 5-5, H4H14706P2 and H4H14708P2 antibodies showed significant and comparable efficacy in reducing IMQ-induced skin thickness and pathology lesion scores in DITRA-like mice. Therapeutic administration of H4H14706P2 and H4H14708P2 led to a significant inhibition of IMQ-induced production of pro-inflammatory cytokines in the skin of DITRA-like mice (Table 5-6).

TABLE 5-4
Therapeutic administration of anti-human IL-36R antibodies reduced
skin thickness in chronic IMQ-induced skin inflammation. Thickness is
presented in μm. Statistical significance within the groups was determined
by one-way Anova with Tukey's multiple comparison post-test and
standard error of mean (SEM±) calculated: ^{#}significantly different
from Vaseline-treated group; *significantly different from PBS-
and Isotype-treated groups. N = 9/group.

| Vaseline | IMQ | | | |
|---|---|---|---|---|
| PBS | PBS | H4H14706P2 | H4H14708P2 | hlgG4 Isotype |
| 505 ± 70 | 953 ± 74^{####} | 667 ± 50**** | 674 ± 38**** | 951 ± 56.7^{####} |

p value: ^{#,}*p < 0.01; ^{##},**p < 0.001; ^{###,}***p < 0.001; ^{####,}****p < 0.0001

TABLE 5-5
Therapeutic administration of anti-human IL-36R antibodies reduced
total pathology score in chronic IMQ-induced skin inflammation.
Statistical significance within the groups was determined by one-way
Anova with Tukey's multiple comparison post-test and standard error of
mean (SEM±) calculated: ^{#}significantly different from Vaseline-treated
group; *significantly different from PBS- and Isotype-treated
groups. N = 9/group.

| Vaseline | IMQ | | | |
|---|---|---|---|---|
| PBS | PBS | H4H14706P2 | H4H14708P2 | hlgG4 Isotype |
| 0 | 17.2 ± 2.9^{####} | 12.5 ± 2.2* | 9.6 ± 1.9*** | 18 ± 2.7^{####} |

p value: ^{#,}*p < 0.01; ^{##,}**p < 0.001; ^{###,}***p < 0.001; ^{####,}****p < 0.0001

TABLE 5-6
hIL-36R antagonism significantly inhibited pro-inflammatory
cytokines in chronic IMQ-induced skin inflammation. Cytokine
levels in PBS/Vaseline control groups were subtracted from all the
treatment groups. Statistical significance within the groups was
determined by one-way Anova with Tukey's multiple comparison
post-test and standard error of mean (SEM±) calculated: *significantly
different from PBS- and Isotype-treated groups. N = 9/group.

| | IMQ | | | |
|---|---|---|---|---|
| | PBS | H4H14706P2 | H4H14708P2 | hlgG4 Isotype |
| KC-GRO | 4.5 ± 1.7 | 0.6 ± 0.3**** | 0.8 ± 0.4**** | 4.4 ± 2.9 |
| IL-6 | 21.1 ± 6.7 | 5.1 ± 1**** | 6.8 ± 1.4**** | 22.9 ± 13.9 |
| IL-1β | 29.4 ± 11.6 | 1.8 ± 0.9**** | 1.9 ± 0.7**** | 23.6 ± 19.4 |
| TNF-α | 12 ± 2.7 | 2.1 ± 0.8**** | 1.8 ± 0.4**** | 14.4 ± 9.3 |

p value: ^{#,}*p < 0.01; ^{##,**}p < 0.001; ^{###,}***p < 0.001; ^{####,}****p < 0.0001

Altogether, these data demonstrated prophylactic and therapeutic efficacy of anti-human IL-36R antibodies in ameliorating IMQ-induced skin inflammation in vivo. H4H14706P2 and H4H14708P2 antibodies displayed comparable ability to significantly reduce both acute and chronic IMQ-induced skin pathology in DITRA-like mice.

Anti-human IL-36R monoclonal antibodies ameliorate DSS-induced chronic colitis in DITRA-like mice at therapeutic dosing. To explore the role of IL-36R antagonism in intestinal inflammation, a chemical model of intestinal injury was used. This model utilized oral administration of DSS that damaged the colonic epithelium (Okayasu et al., A novel model in the induction of reliable experimental and chronic ulcerative colitis in mice, Gastroenterology 1990, 98:694-702) and triggered potent inflammatory responses (Rakoff-Nahoum et al., Recognition of commensal microflora by toll-like receptors is required for intestinal homeostasis. Cell 2004, 118: 229-241) exhibiting the main features of IBD—in particular ulcerative colitis. DITRA-like mice were subjected to chronic DSS-induced colitis by administration of 2-3% DSS for 7 days followed by 10 days of water for two cycles. H4H14706P2 and H4H14708P2 antibodies were administered at 10 mg/kg and 5 mg/kg bi-weekly starting on d7. Control groups received PBS and hIgG4 Isotype control intraperitoneal injections at 10 mg/kg. To monitor intestinal inflammation at different stages of the disease, feces from individual mice were collected on a weekly basis to measure fecal Lipocalin-2 (Lcn2) protein, a non-invasive biomarker of inflammation in intestinal injury (Thorsvik et al., Fecal neutrophil gelatinase-associated lipocalin as a biomarker for inflammatory bowel disease. J Gastroenterol Hepatol 2017, 32:128-135). As shown in Table 5-7, PBS- and hIgG4-treated groups displayed significant upregulation of fecal Lcn2 levels on d12, 19 (not shown) and 28 compared to water alone. On the contrary, two therapeutic administrations of H4H14706P2 and H4H14708P2 resulted in a significant reduction in Lcn2 levels in a dose-dependent manner on d12 compared to PBS- and Isotype-treated groups. Sustained reduction of fecal Lcn2 levels was observed in anti-human IL-36 antibody-treated groups at d19 (not shown) and d28 supporting a role for anti-IL-36R antibodies in reducing intestinal inflammation in DITRA-like mice (Table 5-7). H4H14706P2 antibody displayed better ability to reduce Lcn2 levels and, thus, intestinal inflammation, compared to H4H14708P2 (Table 5-7).

TABLE 5-7
hIL-36R antagonism significantly reduced fecal Lcn2 levels in DITRA-like mice in
chronic DSS-induced colitis. Statistical significance within the groups was determined by one-
way Anova with Tukey's multiple comparison post-test and standard error of mean (SEM±)
calculated: ^{#}significantly different from water-treated group; *significantly different from PBS
and Isotype-treated groups. N = 6-8/group.

| | | DSS | | | | | | |
|---|---|---|---|---|---|---|---|---|
| | | | Day 12 | | | | | |
| Water | | | | H4H14706P2 | | H4H14708P2 | | hIg4 Isolype |
| d0 | d28 | d0 | PBS | 10 mg/kg | 5 mg/kg | 10 mg/kg | 5 mg/kg | 10 mg/kg |
| 0 | 0 | 0 | 1502 ± 525^{####} | 332 ± 107**** | 544 ± 153**** | 698 ± 272* | 791 ± 5.7 | 1879 ± 138^{####} |

| DSS | | | | | |
|---|---|---|---|---|---|
| Day 28 | | | | | |
| | H4H14706P2 | | H4H14708P2 | | hIg4 Isolype |
| PBS | 10 mg/kg | 5 mg/kg | 10 mg/kg | 5 mg/kg | 10 mg/kg |
| 1379 ± 390^{###} | 325 ± 134** | 373 ± 217* | 635 ± 141 | 600 ± 23* | 1448 ± 386^{###} |

p value:
^{#,} *p < 0.01;
^{##,} **p < 0.001;
^{###,} ***p < 0.001;
^{####,} ****p < 0.0001

hIL-36R blockade with H4H14706P2 and H4H14708P2 antibodies led to a decrease in MPO activity (Table 5-8) and 61-95% reduction in pro-inflammatory cytokines (Table 5-9) in the colon DSS-treated DITRA-like mice.

TABLE 5-8
Therapeutic administration of anti-human IL-36R antibodies decreased MPO
activity in the colon of DSS-treated DITRA-like mice. MPO levels are presented as ng per mg
of total protein. Statistical significance within the groups was determined by one-way Anova
with Tukey's multiple comparison post-test and standard error of mean (SEM±) calculated:
^{#}significantly different from water-treated group; *significantly different from PBS- and Isotype-
treated groups. N = 6-8/group.

| | DSS | | | | | |
|---|---|---|---|---|---|---|
| Water | | H4H14706P2 | | H4H14708P2 | | hIgG4 Isotype |
| PBS | PBS | 10 mg/kg | 5 mg/kg | 10 mg/kg | 5 mg/kg | 10 mg/kg |
| 0 | 69 ± 91^{####} | 6.1 ± 2.3*** | 20.5 ± 6.1*** | 29.6 ± 7.5** | 23.5 ± 12.7** | 64.7 ± 5.6^{###} |

p value:
^{#,} *p < 0.01;
^{##,} **p < 0.001;
^{###,} ***p < 0.001;
^{####,} ****p < 0.0001

TABLE 5-9
Therapeutic administration of anti-human IL-36R antibodies decreased pro
inflammatory cytokines in the colon of DSS-treated DITRA-like mice. MPO levels are
presented as no per mg of total protein. Statistical significance within the groups was
determined by one-way Anova with Tukey's multiple comparison post-test and standard error
of mean (SEM±) calculated: #significantly different from water-treated group: * significantly
different from PBS- and Isotype-treated groups. N = 6-8/group.

| Cytokines | | DSS | | | | | |
|---|---|---|---|---|---|---|---|
| (pg per mg of | Water | | H4H14706P2 | | H4H14708P2 | | hIgG4 Isotype |
| total protein) | PBS | PBS | 10 mg/kg | 5 mg/kg | 10 mg/kg | 5 mg/kg | 10 mg/kg |
| KC-GRO | 0.54 ± 0.3 | 98 ± 30#### | 14.7 ± 3*** | 22.9 ± 8** | 38.2 ± 26* | 26.4 ± 0.7* | 110 ± 12### |
| IL-6 | 0.69 ± 0.2 | 345 ± 155#### | 17.1 ± 6**** | 93 ± 75** | 69 ± 11*** | 59 ± 3.4*** | 627 ± 250### |
| IL-1β | 1.2 ± 0.3 | 128 ± 17#### | 13.4 ± 6*** | 27 ± 11** | 42 ± 46* | 34 ± 11* | 125 ± 22### |
| THF-α | 0.98 ± 0.3 | 74 ± 14#### | 9.2 ± 4.7**** | 16 ± 8.8**** | 7.7 ± 4**** | 4.3 ± 2.1**** | 28 ± 15### |

p value:
^{#,} *p < 0.01;
^{##,} **p < 0.001;
^{###,} ***p < 0.001;
^{####,} ****p < 0.0001

Consistent with observations of more reduced Lcn2 levels, H4H14706P2 antibody displayed superior efficacy in reducing MPO activity and pro-inflammatory cytokines in the colon compared to H4H14708P2.

### Example 6. Epitope Mapping of H4H14706P2, H4H14708P2, and H4H14731P Binding to IL-36R by Hydrogen Deuterium Exchange

Hydrogen Deuterium exchange epitope mapping with mass spectrometry (HDX-MS) was performed to determine the amino acid residues of IL-36R (a recombinant human IL-36R designated as hIL-36R.mmH and having the amino acid sequence as set forth in SEQ ID NO: 227) interacting with H4H14706P2, H4H14708P2, and H4H14731P (anti-hIL-36R monoclonal antibodies). A general description of the H/D exchange method is set forth in e.g., Ehring (1999) Analytical Biochemistry 267(2):252-259; and Engen and Smith (2001) Anal. Chem. 73:256A-265A.

The HDX-MS experiments were performed on an integrated HDX/MS platform, consisting of a Leaptec HDX PAL system for the deuterium labeling and quenching, a Waters Acquity M-Class (Auxiliary solvent manager) for the sample digestion and loading, a Waters Acquity M-Class (gBinary solvent manager) for the analytical gradient, and Thermo Q Exactive HF mass spectrometer for peptide mass measurement.

The labeling solution was prepared as PBS buffer in D₂O at pD 7.0 (10 mM phosphate buffer, 140 mM NaCl, and 3 mM KCl, equivalent to pH 7.4 at 25° C.). For deuterium labeling, 11 L of IL-36R.mmH (REGN2105, 45.6 pM in H4H14706P2 and H4H14708P2 experiments, or 63.3 M in H4H14731P experiment) or IL-36R.mmH premixed with H4H14706P2, H4H14708P2, or H4H14731P in 1:0.7 molar ratio (Ag-Ab complex) was incubated at 20° C. with 44 μL D₂O labeling solution for various time-points in duplicate (e.g., Undeuterated control=0 second; deuterium-labeled for 5 minutes and 10 minutes). The deuteration reaction was quenched by adding 55 μL of pre-chilled quench buffer (0.5 M TCEP-HCl, 8 M urea and 1% formic acid) to each sample for a 5-minute incubation at 20° C. The quenched sample was then injected into a Waters HDX Manager for online pepsin/protease XIII digestion. The digested peptides were separated by a C8 column (1.0 mm×50 mm, NovaBioassays) with a 13-minute gradient from 10%-32% B (mobile phase A: 0.5% formic acid in water, mobile phase B: 0.1% formic acid in acetonitrile). The eluted peptides were analyzed by Q Exactive HF mass spectrometry in LC-MS/MS or LC-MS mode.

The LC-MS/MS data of undeuterated IL-36R sample were searched against a database including IL-36R and its randomized sequence using Byonic search engine (Protein Metrics). The search parameters (in ELN) were set as default using non-specific enzymatic digestion and human glycosylation as common variable modification. The list of identified peptides was then imported into the HDX Workbench software (version 3.3) to calculate the deuterium uptake of each peptide detected by LC-MS from all deuterated samples. For a given peptide, the centroid mass (intensity-weighted average mass) at each time point was used to calculate the deuterium uptake (D) and percentage of deuterium uptake (% D).

$$Deuterium Uptake ( D - uptake ) = Average Mass ( deuterated ) - Average Mass ( undeuterated )$$

$$\begin{matrix}Percentage of deuterium uptake \\ ( \% D )\end{matrix} = \frac{D - uptake for peptide at each time point \times 100 \%}{Maximum D - uptake of the peptide ( defined in E L N )}$$

A total of 163 peptides from REGN2105 (hIL-36R.mmH) were identified from both hIL-36R.mmH alone and hIL-36R.mmH in complex with H4H14706P2 samples, representing 81.5% sequence coverage of hIL-36R. Any peptide which exhibited a differential percent D-uptake value above 5% was defined as significantly protected. Peptides corresponding to amino acids 113-122 (YKQILHLGKD) (SEQ ID NO: 229) (amino acids 113-122 of SEQ ID NO: 227) on REGN2105 were significantly protected by H4H14706P2.

A total of 148 peptides from REGN2105 (hIL-36R.mmH) were identified from both hIL-36R.mmH alone and hIL-36R.mmH in complex with H4H14708P2 samples, representing 80.1% sequence coverage of hIL-36R. Any peptide which exhibited a differential percent D-uptake value above 5% was defined as significantly protected. Peptides corresponding to amino acids 113-122 (YKQILHLGKD) (SEQ ID NO: 229) (amino acids 113-122 of SEQ ID NO: 227) on REGN2105 were significantly protected by H4H14708P2.

A total of 237 peptides from REGN2105 (hIL-36R.mmH) were identified from both hIL-36R.mmH alone and hIL-36R.mmH in complex with H4H14731P samples, representing 88.2% sequence coverage of hIL-36R. Any peptide which exhibited a differential percent D-uptake value above 5% was defined as significantly protected. Peptides corresponding to amino acids 264-277 (GVETHVSFREHNLY) (SEQ ID NO: 230) (amino acids 264-277 of SEQ ID NO: 227) on REGN2105 were significantly protected by H4H14731P.

TABLE 6-1
IL-36R.mmH peptides with significant protection upon binding to H4H14706P2

| | | 5 min | | | 10 min | | | |
|---|---|---|---|---|---|---|---|---|
| | | | REGN2105 + | | | REGN2105 + | | |
| | | REGN2105 | H4H14706P2 | | REGN2105 | H4H14706P2 | | |
| IL-36R | Charge | Centroid | Centroid | | Centroid | Centroid | | |
| Residues | (+) | MH⁺ | MH⁺ | ΔD | MH⁺ | MH⁺ | ΔD | Δ% D |
| 113-119 | 2 | 918.84 | 918.60 | −0.24 | 918.97 | 918.71 | −0.26 | −6.2 |
| 113-122 | 1 | 1218.76 | 1218.29 | −0.47 | 1218.91 | 1218.45 | −0.46 | −7.2 |
| 113-122 | 2 | 1219.97 | 1219.52 | −0.45 | 1220.10 | 1219.66 | −0.44 | −6.9 |
| 116-119 | 1 | 497.13 | 497.01 | −0.12 | 497.19 | 497.03 | −0.17 | −8.9 |
| 116-122 | 1 | 798.29 | 797.98 | −0.31 | 798.35 | 797.99 | −0.35 | −8.3 |

TABLE 6-2
IL-36R.mmH peptides with significant protection upon binding to H4H14708P2

| | | 5 min | | | 10 min | | | |
|---|---|---|---|---|---|---|---|---|
| | | | REGN2105 + | | | REGN2105 + | | |
| | | REGN2105 | H4H14708P2 | | REGN2105 | H4H14708P2 | | |
| IL-36R | Charge | Centroid | Centroid | | Centroid | Centroid | | |
| Residues | (+) | MH⁺ | MH⁺ | ΔD | MH⁺ | MH⁺ | ΔD | Δ% D |
| 113-119 | 2 | 918.84 | 918.59 | −0.25 | 918.97 | 918.69 | −0.28 | −6.6 |
| 113-122 | 1 | 1218.73 | 1218.13 | −0.61 | 1218.90 | 1218.31 | −0.58 | −9.3 |
| 113-122 | 2 | 1219.97 | 1219.51 | −0.46 | 1220.10 | 1219.58 | −0.51 | −7.6 |
| 116-119 | 1 | 497.13 | 497.01 | −0.12 | 497.19 | 497.03 | −0.17 | −9.0 |
| 116-122 | 1 | 798.29 | 797.90 | −0.39 | 798.35 | 797.93 | −0.42 | −10.1 |

TABLE 6-3
IL-36R.mmH peptides with significant protection upon binding to H4H14731P

| | | 5 min | | | 10 min | | | |
|---|---|---|---|---|---|---|---|---|
| | | | REGN2105 + | | | REGN2105 + | | |
| | | REGN2105 | H4H14731P | | REGN2105 | H4H14731P | | |
| IL-36R | Charge | Centroid | Centroid | | Centroid | Centroid | | |
| Residues | (+) | MH⁺ | MH⁺ | ΔD | MH⁺ | MH⁺ | ΔD | Δ% D |
| 264-271 | 2 | 880.18 | 879.93 | −0.24 | 880.24 | 879.91 | −0.32 | −5.9 |
| 267-271 | 1 | 592.76 | 592.58 | −0.18 | 592.78 | 592.56 | −0.23 | −8.5 |
| 268-271 | 1 | 491.44 | 491.23 | −0.21 | 491.47 | 491.22 | −0.25 | −14.4 |
| 268-276 | 3 | 1144.59 | 1144.25 | −0.34 | 1144.60 | 1144.21 | −0.39 | −6.6 |
| 268-277 | 3 | 1307.97 | 1307.56 | −0.41 | 1308.01 | 1307.48 | −0.53 | −7.3 |
| 271-276 | 2 | 818.86 | 818.67 | −0.19 | 818.86 | 818.63 | −0.23 | −6.5 |

Amino acid sequence of recombinant human IL-36R (IL1RL2; interleukin 1 receptor-like 2; REGN2105) (hIL36R.mmH): Monomeric human IL-36R (amino acids D20-Y337, Accession #Q9HB29), with a C-terminal myc-myc-hexahistidine (mmH) tag (underlined):

(SEQ ID NO: 227)
DGCKDIFMKNEILSASQPFAFNCTFPPITSGEVSVTWYKNSSKIPVSKII
QSRIHQDETWILFLPMEWGDSGVYQCVIKGRDSCHRIHVNLTVFEKHWCD
TSIGGLPNLSDEYKQILHLGKDDSLTCHLHFPKSCVLGPIKWYKDCNEIK
GERFTVLETRLLVSNVSAEDRGNYACQAILTHSGKQYEVLNGITVSITER
AGYGGSVPKIIYPKNHSIEVLQLGTTLIVDCNVTDTKDNTNLRCWRVNNT
LVDDYYDESKRIREGVETHVSFREHNLYTVNITFLEVKMEDYGLPFMCHA
GVSTAYIILQLPAPDFRAYEQKLISEEDLGGEQKLISEEDLHHHHHH.

### Example 7: In Vivo Evaluation of Anti-IL36R in IMQ-Induced and Oxazolone-Induced Skin Inflammation and Chronic Colitis Mouse Models

The anti-human IL-36R monoclonal antibodies of the present invention were tested in primary human cell assays in vitro; and compared with other anti-human IL-36R monoclonal antibodies in in vivo Imiquimod (IMQ)-induced skin inflammation assays in humanized IL-36R/hIL-36α, β, γ mice. Also, the anti-human IL-36R monoclonal antibodies of the present invention were tested in vivo in an oxazolone-induced model of colitis in humanized IL-36R/hIL-36α, β, γ mice.

IL-8 was detected in culture supernatants using DuoSet ELISA kit for Human CXCL8/IL-8 (R&D Systems) and cytokine was detected in skin and colon homogenates using Proinflammatory Panel 1 (mouse and human) Multiplex Immunoassay kit (MSD). Monoclonal antibodies tested were H4H14706P2, H4H14708P2, APE6155 (IgG4) and a human IgG4 isotype control (REGN1002).

The APE6155 heavy chain (comprising an IgG4 constant domain) comprises the amino acid sequence:

(SEQ ID NO: 239)
QVQLVQSGAEVKKPGASVKVSCKASGYTFTNYWMNWVRQAPRQGLEWMGM
FHPTGDVTRLNQKFKDRVTMTRDTSTSTVYMELSSLRSEDTAVYYCARTT
SMIIGGFAYWGQGLTVTVSSASTKGPSVFPLAPCSRSTSESTAALGCLVK
DYFPEPVTVSWNSGALTSGVHTFAPVLQSSGLYSLSSVVTVPSSSLGTKT
YTCNVDHKPSNTKVDKRVESKYGPPCPPCPAPPVAGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK

The APE6155 light chain (comprising a Kappa constant domain) comprises the amino acid sequence:

(SEQ ID NO: 240)
DIVMTQTPLSLSVTPGQPASISVRSSKSLLHRNAITYFYWYLHKPGQPPQ
LLIYQMSNLASGVPDRFSGSGSGTDFTLKISRVEAEDVGVYYCAQNLELP
LTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

See WO2016/168542.

Testing anti-human IL-36R antibodies in vitro in primary human cells assays in vitro. Normal Human Epidermal Keratinocytes (NHLF; Lonza, Cat #00192627, lot #254498) and Intestinal MyoFibroblast (InMyoFib; Lonza, Cat #CC-2902, lot #0000254498) were cultured in vitro for 4-5 passages in KGM-Gold™ supplemented with BulletKit™ (Lonza, Cat #CC-00192060, lot #0000484385) and SmGm™-2 supplemented with BulletKit™ (Lonza, Cat #CC-3182, lot #00004736694), respectively. Human CD14+ monocytes were isolated from peripheral blood of 3 different donors using EasySep Human Monocyte Isolation Kit (StemCell, Cat #19359) per manufacturer's instructions. A day before the assay, primary human cells were plated in corresponding Media at 10000 per well in 96-well flat bottom plate and incubated overnight at 37° C. Cells were stimulated in the presence of constant concentration (10 nM) or serially diluted (starting from 1500 nM) rhIL-36α/IL-1F6 [aa6-158] (R&D Systems, Cat #6995-IL-010/CF, lot #DAFZ0313051), rhIL-36β/IL-1F8 [aa5-157] (R&D Systems, Cat #6834-IL-010/CF, lot #DAKU0514062 and rhIL-367/IL-1F9 [aa18-169] (R&D Systems, Cat #6835-IL-010/CF, lot #DAPK0215011) alone or in combination. Serial dilutions starting from 2400 nM of anti-human IL-36R antibodies were added to the wells. Plates were incubated for 24 hours at 37° C. and supernatants were collected to measure IL-8 using DuoSet ELISA Development System for Human CXCL8/IL-8 (R&D Systems, Cat #DY208-05, lot #325963). To obtain EC₅₀ and IC₅₀ values, the results were analyzed using nonlinear regression (4-parameter logistics) in GraphPad Prism™ software.

Testing and comparing anti-human IL-36R antibodies in IMQ-induced skin inflammation. To induce skin inflammation, 8-10 weeks old humanized DITRA-like female mice had their back hair shaved using mouse hair trimmer (Oster, MiniMax, Cat #78049-100) and skin depilated with 0.5 g Veet hair removal gel three days prior to IMQ cream application. A daily topical dose of 62.5 mg of commercially available IMQ cream (5%) (Aldara, GM Health Care Limited, NDC 99207-206-12, lot #QJ044A) or Vaseline (CVS Pharmacy, NDC 59779-902-88) was applied on the shaved back skin of the mice for four consecutive days. A daily topical dose of 62.5 mg of Aldara translated into a daily dose of 3.125 mg of an active compound. Anti-human IL-36R antibodies—H4H14706P2, H4H14708P2 and APE6155 (IgG4), were administered subcutaneously into back skin at 10 mg/kg on −3d and d1. Control group received PBS and 10 mg/kg of hIgG4 Isotype control (REGN1002) injections. Two or three days after the start of IMQ application, the back skin of the mice started to display signs of erythema, scaling and thickening. The severity of inflammation was measured on a daily basis using an adapted version of the clinical Psoriasis Area and Severity Index. Erythema, scaling and thickening were scored independently on a scale from 0-4: 0, none; 1, slight; 2, moderate; 3, marked; and 4, very marked (van der Fits et al. Imiquimod-induced psoriasis-like skin inflammation in mice is mediated via the IL-23/IL-17 axis. J Immunol 2009, 182:5836-5845). Skin thickness was measured using caliper on d5 (Kaefer).

Histopathology. Skin tissues of 6 mm diameter from murine back were fixed in 10% buffered formalin, and 4-5 μm paraffin embedded sections were stained with hematoxylin and eosin. Skin sections were evaluated blindly for the presence of parakeratosis, orthokeratosis, Munro's microabscess, acanthosis, epidermal ulceration, inflammation in the dermis and hypodermis, blood vessel congestion in the dermis and hypodermis, follicular hyperkeratosis and epithelial hyperplasia. A 0-4 scoring scale was used: 0-within normal limits, 1-minimal, 2-mild, 3-moderate and 4-severe. A total pathology score was calculated for each mouse by adding the individual histopathological feature scores. Data analysis was performed using GraphPad Prism™ software.

Measurement of cytokines in skin homogenates. Full thickness skin tissues of 6 mm diameter from murine back were taken and placed in 15 mL tube containing T-per buffer (Thermo Scientific, Cat #378510, lot #RF236217), 1× Halt Protease Inhibitor Cocktail (Thermo Scientific, Cat #87786, lot #QG221763) and 5 M EDTA Solution (Thermo Scientific, Cat3 78429). Skin tissues were disrupted at 28000 rpm for 1 minute using a Polytron (PT10-35 GT-D, Cat #9158158) and put on ice. Generated skin homogenates were centrifuged at 1500 rpm for 8 minutes at 4° C. and the supernatants were collected into 96-well plates. Skin homogenates were subjected to Bradford protein assay using protein assay dye (BioRad, Cat #500-0006, lot #210008149) to quantify the total protein content. Cytokine concentrations in the skin homogenates were measured using a Proinflammatory Panel 1 (mouse) multiplex immunoassay kit (MesoScale Discovery, Cat #K15048D) according to manufacturer's instructions. In brief, 50 L/well of calibrators and samples (diluted in Diluent 41) were added to the plates pre-coated with capture antibodies and incubated at room temperature while shaking at 700 rpm for 2 hours. The plates were then washed 3 times with 1×PBS containing 0.05% (w/v) Tween-20, followed by the addition of 25 μL of Detection Antibody Solution diluted in Diluent 45. After 2-hours incubation at room temperature while shaking, the plates were washed 3 times, and 150 μL of 2× Read Buffer was added to each well. Electrochemiluminescence was immediately read on a MSD Spector® instrument. Data analysis was performed using GraphPad Prism™ software. Cytokine levels were normalized to total protein content.

Testing anti-human IL-36R monoclonal antibodies in oxazolone-induced intestinal inflammation—Induction of oxazolone-induced model of chronic colitis and antibody treatment in DITRA-like mice. Oxazolone colitis was induced as previously described (Heller et al., Oxazolone colitis, a Th2 colitis model resembling ulcerative colitis, is mediated by IL-13-producing NK-T cells. Immunity 2002, 17: 629-638). Briefly, in order to pre-sensitize DITRA-like mice, a 2×2 cm² field of the abdominal skin was shaved, and 100 μl of a 3% solution oxazolone ((4-ethoxymethylene-2-phenyl-2-oxazoline-5-one; Sigma Aldrich) diluted in 100% ethanol was applied. On days 5 and 7 after pre-sensitization, mice were challenged intrarectally with 50 μl of 1.5% oxazolone diluted in 50% ethanol under general anesthesia. Control mice were pre-sensitized with 100% ethanol and received intrarectal injection of 50% ethanol. Anti-human IL-36R antibodies—H4H14706P2 and H4H14708P2, were administered intraperitoneally at 10 mg/kg on d2, 5 and 7. Control group received PBS and 10 mg/kg of hIgG4 Isotype control (REGN1002) injections. Mice were weighted and monitored for clinical signs of colitis (e.g., stool consistency and fecal blood) on a daily basis. On d8, mice were euthanized and colons were collected.

Measurement of cytokines in colon homogenates. Pieces of distal part of the colon were taken into 2 mL microcentrifuge tubes containing 2 Tungsten 3 mm Carbide Beads (Qiagen) containing T-per buffer (Thermo Scientific), 1× Halt Protease Inhibitor Cocktail (Thermo Scientific) and 5 M EDTA Solution (Thermo Scientific). Colon tissues were disrupted using Qiagen Tissue Lyser II at frequency of 27.5 s⁻¹ for 10 minutes. Tubes were centrifuged at 1500 rpm for 8 minutes at 4° C. and the supernatants were collected into 96-well plates. All tissue homogenates were subjected to Bradford protein assay using protein assay dye (BioRad) to quantify the total protein content.

Cytokine concentrations in the colon homogenates were measured using a Proinflammatory Panel 1 (mouse) multiplex immunoassay kit (MesoScale Discovery Cat #K15048D) according to manufacturer's instructions. In brief, 50 L/well of calibrators and samples (diluted in Diluent 41) were added to the plates pre-coated with capture antibodies and incubated at room temperature while shaking at 700 rpm for 2 hours. The plates were then washed 3 times with 1×PBS containing 0.05% (w/v) Tween-20, followed by the addition of 25 μL of Detection Antibody Solution diluted in Diluent 45. After 2-hour incubation at room temperature while shaking, the plates were washed 3 times, and 150 μL of 2× Read Buffer was added to each well. Electrochemiluminescence was immediately read on MSD Spector® instrument. Data analysis was performed using GraphPad Prism™ software. Cytokine levels were normalized to total protein content.

Statistical analysis. Statistical significance within the groups was determined by one-way Anova with Tukey's multiple comparison post-test (*p<0.05, **p<0.005, ***p<0.0005, ****p<0.00001).

Results summary and conclusions—Anti-human IL-36R monoclonal antibodies potently inhibit human IL-36R signaling in primary human cells in vitro. Human Epidermal Keratinocytes (NHEK), Human Intestinal Myofibroblasts (InMyoFib) and Peripheral Blood (PB)-derived CD14⁺ Monocytes were stimulated in vitro with 10 nM of IL-36□, □ and □. Serially diluted anti-human IL-36R monoclonal antibodies (H4H14706P2 and H4H14708P2) were added to the cultures, supernatants were collected 24 hours post-incubation and human IL-8 production in response to IL-36 stimulation was measured. The anti-human IL-36R monoclonal antibodies potently inhibit all three IL-36 cytokines in Human Epidermal Keratinocytes, Human Intestinal Myofibroblasts and Peripheral Blood (PB)-derived CD14⁺ Monocytes in vitro with IC₅₀ 1-6 nM (Table 7-1).

TABLE 7-1
Anti-human IL-36R antibodies, H4H14706P2 and H4H14708P2, inhibited
human IL-36α, β and γ in human primary cells in vitro.

| | Normal Epidermal | | | Intestinal MyoFibroblasts | | | | | |
|---|---|---|---|---|---|---|---|---|---|
| Cells | Keratinocytes (NHEK) | | | (InMyoFib) | | | PB-derived CD14⁺ Monocytes | | |
| hIL-36 Ligands | α | β | γ | α | β | γ | α | β | γ |
| EC50[M] | 1.43E−09 | 1.18E−09 | 4.00E−09 | 1.41E−09 | 1.18E−09 | 1.46E−09 | 2.75E−09 | 2.63E−09 | 2.75E−09 |
| Constant for Inhibition | 10 nM | 10 nM | 10 nM | 10 nM | 10 nM | 10 nM | 10 nM | 10 nM | 10 nM |
| Protein/hIL-36R ab | IC50 [M] | IC50 [M] | IC50 [M] | IC50 [M] | IC50 [M] | IC50 [M] | IC50 [M] | IC50 [M] | IC50 [M] |
| H4H14706P2 | 4.42E−09 | 3.62E−09 | 2.11E−09 | 4.89E−09 | 3.62E−09 | 4.59E−09 | 1.15E−09 | 1.74E−09 | 1.58E−09 |
| H4H14708P2 | 5.06E−09 | 5.79E−09 | 3.63E−09 | 5.72E−09 | 5.3E−09 | 5.40E−09 | 2.38E−09 | 2.24E−09 | 1.95E−09 |
| hIgG Ctr (REGN1002) | None | None | None | None | None | None | None | None | None |

Anti-human IL-36R monoclonal antibodies H4H14706P2 and H4H14708P2 are more potent than the APE6155 antibody in inhibiting IMQ-induced skin inflammation in DITRA-like mice. H4H14706P2 and H4H14708P2 and APE6155 anti-human IL-36R monoclonal antibodies were tested head-to-head in IMQ-induced model of psoriasiform dermatitis. IMQ was applied daily to the shaved back skin of DITRA-like mice for four consecutive days. H4H14706P2 and H4H14708P2 and APE6155 antibodies were administered at 10 mg/kg on −3d and d1. Control groups received PBS and hIgG4 Isotype control injections at 10 mg/kg. On d5, skin thickness was measured and tissue harvested for subsequent histopathological evaluation and protein isolation. Both H4H14706P2 and H4H14708P2 antibodies displayed greater potency in significantly decreasing IMQ-induced skin thickness compared to APE6155 (Table 7-2). Histopathological evaluation of the skin lesions revealed a greater reduction in total pathology score including parakeratosis and Munro's microabscess with anti-human IL-36R antibodies treatments (Table 7-3).

TABLE 7-2
Anti-human IL-36R antibodies, H4H14706P2 and H4H14708P2, are
more potent than APE6155 anti-human IL-36R antibody in reducing
skin thickness in IMQ-induced skin inflammation.^{~}

| Vaseline | IMQ | | | | |
|---|---|---|---|---|---|
| PBS | PBS | H4H14706P2 | H4H14708P2 | APE6155 | hlgG4 Isotype |
| 607 ± 18 | 748 ± 45 | 586 ± 34** | 585 ± 1.9** | 13.4 ± 3 | 16 ± 1.5 |

^{~}Thickness is presented in μm. Statistical significance within the groups was determined by one-way Anova with Tukey's multiple comparison post-test and standard error of mean (SEM±) calculated: *significantly different from PBS- and Isotype-treated groups. n = 9/group.

TABLE 7-3
Anti-human IL-36R antibodies, H4H14706P2 and H4H14708P2,
displayed greater potency than APE6155 in reducing the total
pathology score in IMQ-induced skin inflammation.^{$}

| Vaseline | IMQ | | | | |
|---|---|---|---|---|---|
| PBS | PBS | H4H14706P2 | H4H14708P2 | APE6155 | hlgG4 Isotype |
| 2 ± 0.6 | 17 ± 2.5 | 11 ± 1.4** | 13.4 ± 3 | 13.4 ± 3 | 16 ± 1.5 |

^{$}Statistical significance within the groups was determined by one-way Anova with Tukey's multiple comparison post-test and standard error of mean (SEM±) calculated: *significantly different from PBS- and Isotype-treated groups. n = 9/group.

Additionally, human IL-36R blockade with H4H14706P2 and H4H14708P2 antibodies resulted in greater reduction in KC-GRO, IL-6, IL-1β and TNFα production in skin homogenates compared to COMP5382 (Table 7-4).

TABLE 7-4
Anti-human IL-36R antibodies, H4H14706P2 and H4H14708P2,
displayed greater potency than APE6155 in reducing
pro-inflammatory cytokines in the skin.^{∞}

| Cytokines | | | | | |
|---|---|---|---|---|---|
| (pg per mg of | IMQ | | | | |
| total tissue) | PBS | H4H14706P2 | H4H14708P2 | APE6155 | hlgG4 Isotype |
| KC-GRO | 64 ± 10 | 19 ± 5.5** | 23 ± 7** | 40 ± 16 | 65 ± 22 |
| IL-6 | 160 ± 47 | 41 ± 14**** | 51 ± 16**** | 128 ± 59 | 165 ± 87 |
| IL-1β | 128 ± 43 | 8.6 ± 1.9**** | 10 ± 1.3**** | 28 ± 17**** | 117 ± 49 |
| TNF-α | 72 ± 22 | 11 ± 4.2* | 12 ± 2.9* | 20.5 ± 9.8 | 65 ± 22 |

^{∞}Values are presented as “pg per mg of total tissue”. Statistical significance within the groups was determined by one-way Anova with Tukey's multiple comparison post-test and standard error of mean (SEM±) calculated: *significantly different from PBS- and Isotype-treated groups. n = 9/group.

Anti-human IL-36R monoclonal antibodies ameliorate Oxazolone-induced colitis in DITRA-like mice. To further explore biological function of IL-36 in the gut, we tested the efficacy of IL-36R blockade in oxazolone-induced colitis, another preclinical model of IBD with the histologic resemblance to human ulcerative colitis (Heller et al.). Prophylactic administration of anti-human IL-36R antibodies, H4H14706P2 and H4H14708P2, significantly reduced oxazolone-induced disease severity in DITRA-like mice compared to PBS and isotype control treated groups as reflected in levels of IL-4, IL-6 and TNF-α in the colon of oxazolone-treated DITRA-like mice (Table 7-5).

TABLE 7-5
Human IL-36R antagonism ameliorates oxazolone-induced colitis
in DITRA-like mice in vivo.^{♦}

| Cytokines | | | | | |
|---|---|---|---|---|---|
| (pg per mg of | Oxazolone | | | | |
| total tissue) | Vehicle | PBS | H4H14706P2 | H4H14708P2 | hlgG4 Isotype |
| IL-4 | 2 ± 0.4 | 509 ± 148 | 51.5 ± 28**** | 111 ± 35**** | 296 ± 106 |
| IL-6 | 34 ± 29 | 2128 ± 1255 | 198 ± 131** | 214 ± 116** | 2276 ± 1338 |
| TNF-α | 95 ± 36 | 822 ± 149 | 387 ± 126** | 569 ± 136 | 859 ± 148 |

DITRA-like mice were pre-sensitized with 3% solution of oxazolone dissolved in 100% Ethanol and intrarectally administered with 1.5% oxazolone and vehicle (50% Ethanol) 5 days later. Mice were intraperitoneally injected with PBS, anti-human IL-36R mAb and hIgG4 Isotype control on days 2, 5 and 7 after pre-sensitization. Levels of pro-inflammatory cytokines in colon homogenates in oxazolone- and vehicle- treated DITRA-like mice injected with PBS, anti-human IL-36R mAb and hIgG4 Isotype control. Values are presented as “pg per mg of total tissue”. Statistical significance within the groups was determined by one-way Anova with Tukey's multiple comparison post-test and standard error of mean (SEM±) calculated: *represents significant difference from PBS-treated group. n = 5/group.

### Example 8: Bioassay Using Human HEK293/NFkB-Luc/hIL36R Cell Line for Schild Analysis

To characterize the inhibitory properties of the anti-IL36R antibodies, H4H14706P2 and H4H14708P2, a Schild analysis was performed. This method assesses the nature of antagonism by inhibitors and measures the affinity of a competitive antagonist when a number of conditions are fulfilled (Colquhoun, Why the Schild method is better than Schild realized, Trends Pharmacol Sci. 2007 December; 28(12):608-14).

For the bioassay, HEK293/NFκB-luc/hIL-36R cells are seeded onto 96-well assay plates at 10,000 cells/well in low serum media, 0.1% FBS and OPTIMEM, and incubated at 37° C. and 5% CO₂ overnight. Next day, antibody was added to cells at different, fixed concentrations (9 nM, 3 nM, 1 nM, 0.3 nM or 0.1 nM) and pre-incubated with cells for 15 minutes at room temperature. A condition without antibody was also included. IL-36α, IL-36β, or IL-36γ were then serially diluted from 100 nM to 2 pM or 100 nM to 0.1 pM and were added to cells along with sample without any ligand. Luciferase activity was detected after 5.5 hrs of incubation in 37° C. and 5% CO₂ with Victor X5 or EnVision™ Multilabel Plate Reader (Perkin Elmer) and the results were analyzed using Gaddum/Schild EC₅₀ shift with Prism 7 (GraphPad).

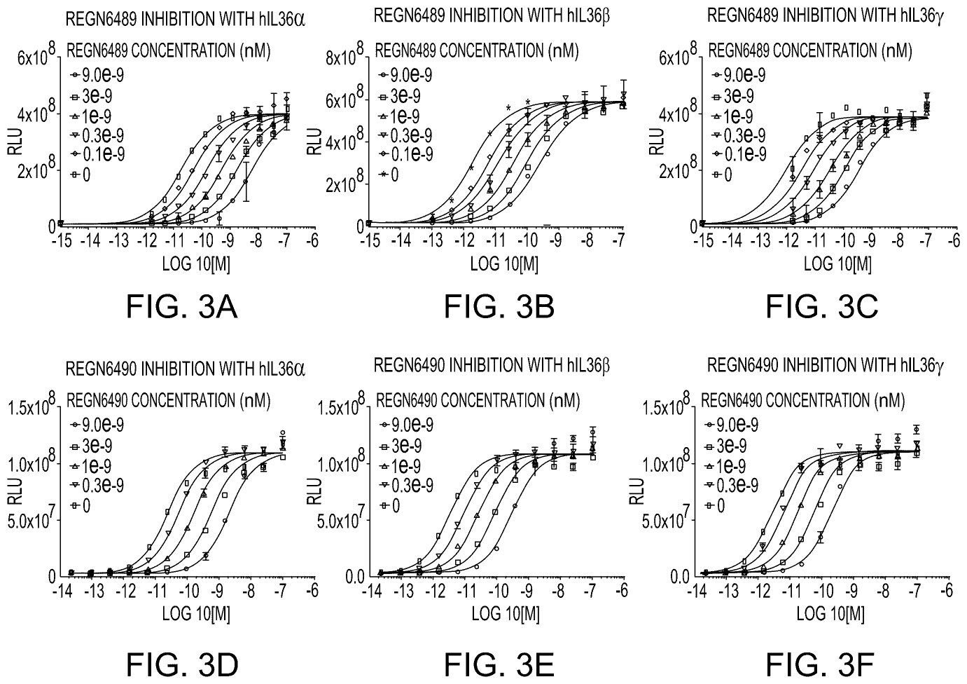

A Schild analysis of H4H14706P2 and H4H14708P2 showed that increasing amount of antibodies caused parallel rightward shift of the IL36 ligand dose-response curves and that the inhibitory effect of H4H14706P2 and H4H14708P2 was surmountable by increasing amounts of IL36 ligand, suggesting competitive inhibition of H4H14706P2 and H4H14708P2 (FIGS. 3A-3F).

### Example 9: Pharmacokinetic (PK) Studies

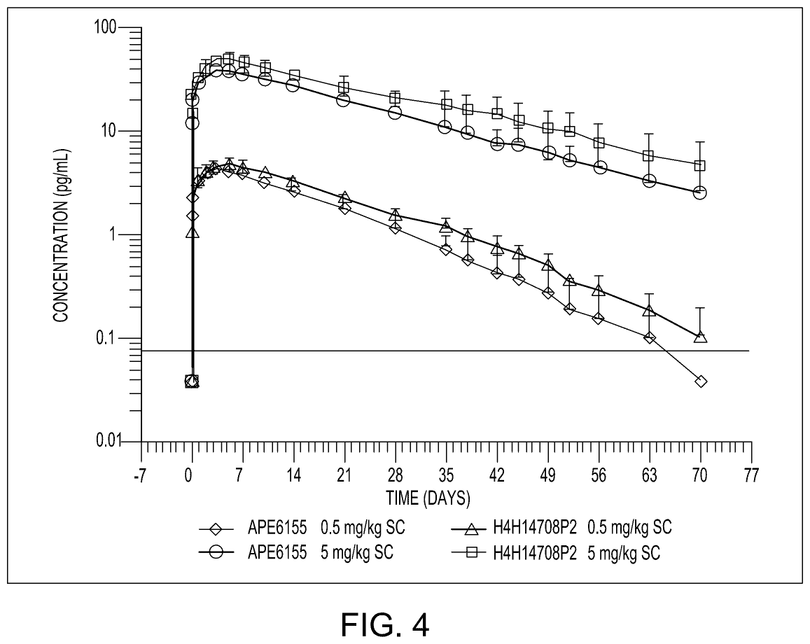

Female cynomolgus monkeys were assigned to dose groups for PK characterization; animals (3 animals/group) received a single SC injection of 5 or 0.5 mg/kg of H4H14708P2 or APE6155. Blood samples were collected from all animals at pre-dose and at 4, 24, 48, 72, 120, 168, 240, 336, 504, 576, 672, 840, 912, 1008, 1080, 1176, 1248, 1344, 1512 and 1680 hours post dose. Concentrations of total H4H14708P2 or APE6155 in serum were determined using a non-validated enzyme-linked immunosorbent assays (ELISAs). The method was designed to measure total human IgG concentrations in cynomolgus serum. Pharmacokinetic parameters were estimated using non-compartmental analysis. H4H14708P2 was observed to have about 1.3-fold greater exposure than APE6155 at 5 mg/kg dosage and about 1.2-fold greater exposure than APE6155 at 0.5 mg/kg dosage. See FIG. 4.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, to relate to each and every individual publication, database entry (e.g., Genbank sequences or GeneID entries), patent application, or patent even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1.-26. (canceled)

27. A method for treating or preventing an IL-36R-mediated disease in a subject in need thereof, comprising administering a therapeutically effective amount of an antibody or antigen-binding fragment thereof which specifically binds to Interleukin-36 Receptor (IL-36R), wherein:
the antibody or antigen-binding fragment thereof comprises:
(i) a heavy chain immunoglobulin variable region that comprises CDR-H1, CDR-H2 and CDR-H3 of a heavy chain immunoglobulin or variable region thereof that comprises the amino acid sequence set forth in SEQ ID NO: 2, 18, 34, 50, 66, 82, 98, 114, 130, 138, 154, 170, 180, 184, 188, 192, 196, 200, 204, 208, 212, 216, 220 or 224; and
(ii) a light chain immunoglobulin or variable region thereof that comprises CDR-L1, CDR-L2 and CDR-L3 of a light chain immunoglobulin or variable region thereof that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226.

28. (canceled)

29. The method of claim 27, wherein the IL-36R-mediated disease is psoriasis.

30.-31. (canceled)

32. The method of claim 27, wherein the subject has a homozygous or heterozygous IL36RN mutation genotype.

33. The method of claim 27, wherein the IL-36R-mediated disease is inflammatory bowel disease.

34. The method of claim 27, wherein the IL-36R-mediated disease is inflammatory bowel disease generalized pustular psoriasis or skin inflammation.

35. The method of claim 27, wherein the antibody or antigen-binding fragment thereof comprises a heavy chain immunoglobulin variable region comprising the CDR-H1, CDR-H2 and CDR-H3 of a heavy chain immunoglobulin variable region that comprises the amino acid sequence set forth in SEQ ID NO: 34, and a light chain immunoglobulin variable region comprising the CDR-L1, CDR-L2 and CDR-L3 of a light chain immunoglobulin variable region that comprises the amino acid sequence set forth in SEQ ID NO: 42.

36. The method of claim 27, wherein the antibody or antigen-binding fragment thereof comprises a heavy chain immunoglobulin variable region comprising the CDR-H1, CDR-H2 and CDR-H3 of a heavy chain immunoglobulin variable region that comprises the amino acid sequence set forth in SEQ ID NO: 50, and a light chain immunoglobulin variable region comprising the CDR-L1, CDR-L2 and CDR-L3 of a light chain immunoglobulin variable region that comprises the amino acid sequence set forth in SEQ ID NO: 58.

37. The method of claim 27, wherein the antibody or antigen-binding fragment thereof comprises:
a heavy chain immunoglobulin or variable region thereof that comprises:
a CDR-H1 comprising the amino acid sequence set forth in SEQ ID NO: 36, a CDR-H2 comprising the amino acid sequence set forth in SEQ ID NO: 38, and a CDR-H3 comprising the amino acid sequence set forth in SEQ ID NO: 40; and
a light chain immunoglobulin or variable region thereof that comprises:
a CDR-L1 comprising the amino acid sequence set forth in SEQ ID NO: 44, a CDR-L2 comprising the amino acid sequence set forth in SEQ ID NO: 46, and a CDR-L3 comprising the amino acid sequence set forth in SEQ ID NO: 48.

38. The method of claim 27, wherein the antibody or antigen-binding fragment thereof comprises:
a heavy chain immunoglobulin or variable region thereof that comprises:
a CDR-H1 comprising the amino acid sequence set forth in SEQ ID NO: 52, a CDR-H2 comprising the amino acid sequence set forth in SEQ ID NO: 54, and a CDR-H3 comprising the amino acid sequence set forth in SEQ ID NO: 56; and
a light chain immunoglobulin or variable region thereof that comprises:
a CDR-L1 comprising the amino acid sequence set forth in SEQ ID NO: 60, a CDR-L2 comprising the amino acid sequence set forth in SEQ ID NO: 62, and a CDR-L3 comprising the amino acid sequence set forth in SEQ ID NO: 64

39. The method of claim 27, wherein the heavy chain immunoglobulin variable region is linked to an IgG, IgG1 or IgG4 heavy chain constant region and the light chain immunoglobulin variable region is linked to a kappa or lambda light chain constant region.

40. The method of claim 27, wherein the antibody or antigen-binding fragment thereof comprises a heavy chain immunoglobulin that comprises the amino acid sequence set forth in SEQ ID NO: 188, and a light chain immunoglobulin that comprises the amino acid sequence set forth in SEQ ID NO: 190.

41. The method of claim 27, wherein the antibody or antigen-binding fragment thereof comprises a heavy chain immunoglobulin that comprises the amino acid sequence set forth in SEQ ID NO: 192, and a light chain immunoglobulin that comprises the amino acid sequence set forth in SEQ ID NO: 194.

42. A method for treating or preventing: an inflammatory disorder, an autoimmune disorder, deficiency of interleukin IL-36 receptor antagonist (DITRA) syndrome, impetigo herpetiformis, acrodermatitis, a skin neutrophilic pustular disease, a pustular disease, psoriasis, generalized pustular psoriasis, psoriasis vulgaris, palmoplantar pustular psoriasis, palmoplantar pustulosis, colitis, airway inflammation, joint inflammation, kidney inflammation, alopecia areata, skin inflammation, acanthosis, hyperkeratosis, kindler syndrome, systemic lupus erythematosus (SLE), nephrotic syndrome, ANCA (anti-neutrophil cytoplasmic antibody)-associated vasculopathies, tubulointerstitial lesions or glomerulonephritis; the method comprising administering a therapeutically effective amount of an antibody or antigen-binding fragment thereof that specifically binds Interleukin-36 Receptor (TL-36R), wherein the antibody or antigen-binding fragment thereof comprises:
(i) a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 2, 18, 34, 50, 66, 82, 98, 114, 130, 138, 154, 170, 180, 184, 188, 192, 196, 200, 204, 208, 212, 216, 220 or 224; or a variant thereof, and/or
(ii) a light chain immunoglobulin or variable region thereof that comprises CDR-L1, CDR-L2 and CDR-L3 of a light chain immunoglobulin or variable region thereof that comprises the amino acid sequence set forth in SEQ ID NO: 10, 26, 42, 58, 74, 90, 106, 122, 146, 162, 182, 186, 190, 194, 198, 202, 206, 210, 214, 218, 222 or 226; or a variant thereof.

43. The method of claim 42, for treating or preventing psoriasis.

44. The method of claim 42, for treating or preventing inflammatory bowel disease.

45. The method of claim 42, for treating generalized pustular psoriasis.

46. The method of claim 42, for treating skin inflammation.

47. The method of claim 42, wherein the subject has a homozygous or heterozygous IL36RN mutation genotype.

48. The method of claim 42, wherein the antibody or antigen-binding fragment thereof comprises a heavy chain immunoglobulin variable region that comprises the amino acid sequence set forth in SEQ ID NO: 34, and a light chain immunoglobulin variable region that comprises the amino acid sequence set forth in SEQ ID NO: 42.

49. The method of claim 42, wherein the antibody or antigen-binding fragment thereof comprises a heavy chain immunoglobulin variable region that comprises the amino acid sequence set forth in SEQ ID NO: 50, and a light chain immunoglobulin variable region that comprises the amino acid sequence set forth in SEQ ID NO: 58.

50. The method of claim 42, wherein the heavy chain immunoglobulin variable region is linked to an IgG, IgG1 or IgG4 heavy chain constant region and the light chain immunoglobulin variable region is linked to a kappa or lambda light chain constant region.

51. The method of claim 42, wherein the antibody or antigen-binding fragment thereof comprises a heavy chain immunoglobulin that comprises the amino acid sequence set forth in SEQ ID NO: 188, and a light chain immunoglobulin that comprises the amino acid sequence set forth in SEQ ID NO: 190.

52. The method of claim 42, wherein the antibody or antigen-binding fragment thereof comprises a heavy chain immunoglobulin that comprises the amino acid sequence set forth in SEQ ID NO: 192, and a light chain immunoglobulin that comprises the amino acid sequence set forth in SEQ ID NO: 194.
